(19) United States
(12) Patent Application Publication
(10) Pub. No.: US 2013/0149308 A1
(43) Pub. Date: Jun. 13, 2013

(54) ANTIBODIES TO IL-1BETA AND IL-18, FOR TREATMENT OF DISEASE

(71) Applicant: Genentech, Inc., South San Francisco, CA (US)

(72) Inventor: Menno van Lookeren Campagne, South San Francisco, CA (US)

(73) Assignee: GENENTECH, INC., South San Francisco, CA (US)

(21) Appl. No.: 13/763,896

(22) Filed: Feb. 11, 2013

Related U.S. Application Data
(63) Continuation of application No. PCT/US2011/047532, filed on Aug. 12, 2011.

(51) Int. Cl. C07K 16/24

(52) U.S. Cl. CPC C07K 16/244; C07K 16/245
USPC 424/136.1; 424/133.1; 424/145.1; 424/158.1; 435/375; 530/387.3; 530/389.2

(57) ABSTRACT

The present invention relates to compositions and methods for treatment of disease. More particularly, the present invention relates to anti-IL-1β and anti-IL-18 antibodies, including anti-IL-1β and anti-IL-18 bispecific antibodies, and methods of treating disease using such antibodies.

### RELATED APPLICATION

This application is a continuation of International Application No. PCT/US2011/047532 having an international filing date of Aug. 12, 2011, which claims the benefit of priority of U.S. Provisional Application Ser. No. 61/373,760 filed 13 Aug. 2010, each of which are incorporated by reference herein in their entirety.

### FIELD OF THE INVENTION

The present invention relates generally to anti-IL-1β and anti-IL-18 antibodies, including anti-IL-1β and anti-IL-18 bispecific antibodies and monoclonal antibodies, and methods of using such antibodies for the treatment of disease.

### BACKGROUND

The interleukin-1 (IL-1) and IL-18 family of cytokines are related by mechanism of origin, receptor structure, and signal transduction pathways utilized. These cytokines are synthesized as precursor molecules and cleaved by the enzyme caspase-1 before or during release from the cell. The NALP-3 inflammasome is of crucial importance in generating active caspase-1 (Cassel et al., 2009; Ferrero-Miliani et al., 2007). The IL-1 family contains two agonists, IL-1α and IL-1β, a specific inhibitor, IL-1 receptor antagonist (IL-1Ra), and two receptors, the biologically active type IL-1R and inactive type II IL-1R (Arend et al., 2008). Both IL-1RI and IL-33R utilize the same interacting accessory protein (IL-1RAcP). The balance between IL-1 and IL-1Ra is important in preventing disease in various organs, and excess production of IL-1 has been implicated in many human diseases. The IL-18 family also contains a specific inhibitor, the IL-18-binding protein (IL-18BP), which binds IL-18 in the fluid phase. The IL-18 receptor is similar to the IL-1 receptor complex, including a single ligand-binding chain and a different interacting accessory protein. IL-18 provides an important link between the innate and adaptive immune responses.

Inflammasome activation and IL-1β/IL-18 processing and secretion may be involved in disease progression. Genome-wide association studies indicate a role for the inflammasome in inflammatory bowel disease (IBD). Patients with polymorphisms in the inflammasome-compound NALP-3 are reportedly at increased risk for Crohn's disease (Ferrero-Miliani et al., 2007; Villani et al., 2009). In addition, polymorphisms in autophagy components Atg16l1 and IRGM that control caspase-1 activation and IL-1β/IL-18 processing have been reportedly linked to Crohn's disease (Baldassano et al., 2007; Cadwell et al., 2008; Kuballa et al., 2008; Saitoh et al., 2008). Independent studies have reported increased serum levels of IL-1β and IL-18 in patients with IBD (Ludwiczek et al., 2005; Ludwiczek et al., 2004; Monteleone et al., 1999). Studies in humans have been further supported by preclinical studies. Blockade of IL-18 or IL-1β reportedly leads to amelioration of clinical scores in preclinical models of the disease (Ten Hove et al., 2001).

Further, it has been reported that in the eye, there are increased levels of IL-1β in patients with diabetic retinopathy (Kowluru and Odenbach, 2004).

### SUMMARY OF THE INVENTION

The present invention provides anti-IL-1μ and anti-IL-18 antibodies, including e.g., anti-IL-1β and anti-IL-18 bispecific antibodies, and methods of using such antibodies for treatment of disease. In some embodiments, the anti-IL-1β and anti-IL-18 antibodies are monoclonal antibodies, and are administered concurrently or consecutively to a patient, for treatment of disease. In other embodiments, the anti-IL-1β and anti-IL-18 are bispecific antibodies and are administered to a patient for treatment of disease. In some embodiments the disease is an inflammasome-mediated disease, e.g., a disease wherein the inflammasome is activated. Examples of diseases include immune diseases and autoimmune diseases, and include inflammatory bowel disease (IBD), age-related macular degeneration (AMD), and type 2 diabetes (T2D).

In some embodiments, the anti-IL-1β and anti-IL-18 antibodies of the present invention, block or neutralize the activity of, and/or bind to, IL-1β and/or IL-18. In some embodiments the bispecific antibody blocks or neutralizes the activity or, and/or binds to, IL-1β and/or IL-18.

In one aspect, there is provided a method of treating a disease in a patient, the method comprising administering to said patient an effective amount of:

a. An IL-1β/IL-18 bispecific antibody; or

b. An antibody that binds IL-1β and IL-18; or

c. An antibody that binds IL-1β and an antibody that binds IL-18;

wherein said antibody or antibodies of parts a, b or c is/are capable of neutralizing or blocking IL-1β and IL-18 activity in cells or tissue.

In some embodiments, the antibody/antibodies used in the method is/are humanized. In some embodiments, the antibody is a dual action antibody.

In some embodiments, the method uses a combined treatment comprising an anti-IL-1β antibody and an anti-IL-18 antibody. In one embodiment, at least one antibody is monoclonal. In some embodiments, each antibody is monoclonal. In some embodiments, the antibodies of part (c) are given simultaneously, or consecutively. In some embodiments, the antibodies are administered within one hour.

In some embodiments, the disease to be treated is an immune disease or an autoimmune disease or an inflammatory or an autoinflammatory disease. In some embodiments, the disease is an inflammasome-mediated disease. In some embodiments, the disease is an IL-1β related disease or an IL-18 related disease or an IL-1β/IL-18 β disease.

In some embodiments, the disease is age-related macular degeneration (AMD). In some embodiments, the disease is type 2 diabetes (T2D). In some embodiments, the inflammatory bowel disease (IBD). In some embodiments, the disease is Crohn's disease (CD). In some embodiments, the disease is ulcerative colitis (UC). In some embodiments, the disease is atherosclerosis. In some embodiments, the disease is cardio-metabolic disease. In some embodiments, the disease is fibrostenosing Crohn's disease.

In some embodiments, the patient being treated by the method has not responded to anti-TNF therapy.

In some embodiments, the method of treating disease in a patient comprises administering to said patient an effective amount of a monoclonal antibody that binds IL-1β and a monoclonal antibody that binds IL-18.

In another aspect, there is provided a method of neutralizing or blocking IL-1β and/or IL-18 activity in cells or tissue, the method comprising contacting said cells or tissue with a monoclonal antibody that binds IL-1β and a monoclonal antibody that binds IL-18, and thereby neutralizing or blocking said activity. In some embodiments, the antibodies are administered concurrently or consecutively. In some embodiments, the cells are contacted concurrently or consecutively with said monoclonal antibody that binds IL-1β and said monoclonal antibody that binds IL-18.

In another aspect, there is provided an antibody that neutralizes or blocks IL-1β and IL-18 activity. In some embodiments, the antibody is a bispecific antibody. In some embodiments, the antibody is humanized. In some embodiments, the antibody binds to IL-1β and IL-18.

### BRIEF DESCRIPTION OF THE DRAWINGS

FIG. 1 depicts examples of ligands and receptors for IL-1β and for IL-18. For example, signaling can be initiated though engagement of two receptor chains by IL-1β or IL-18. It is thought that intracellular Toll-Interleukin Rexeptor-like (TIR) domain leads to activation of transcription factors NF-kB and AP1 that in turn increase cytokine production ultimately resulting in protective immunity, autoinflammatory disorders or chronic inflammation.

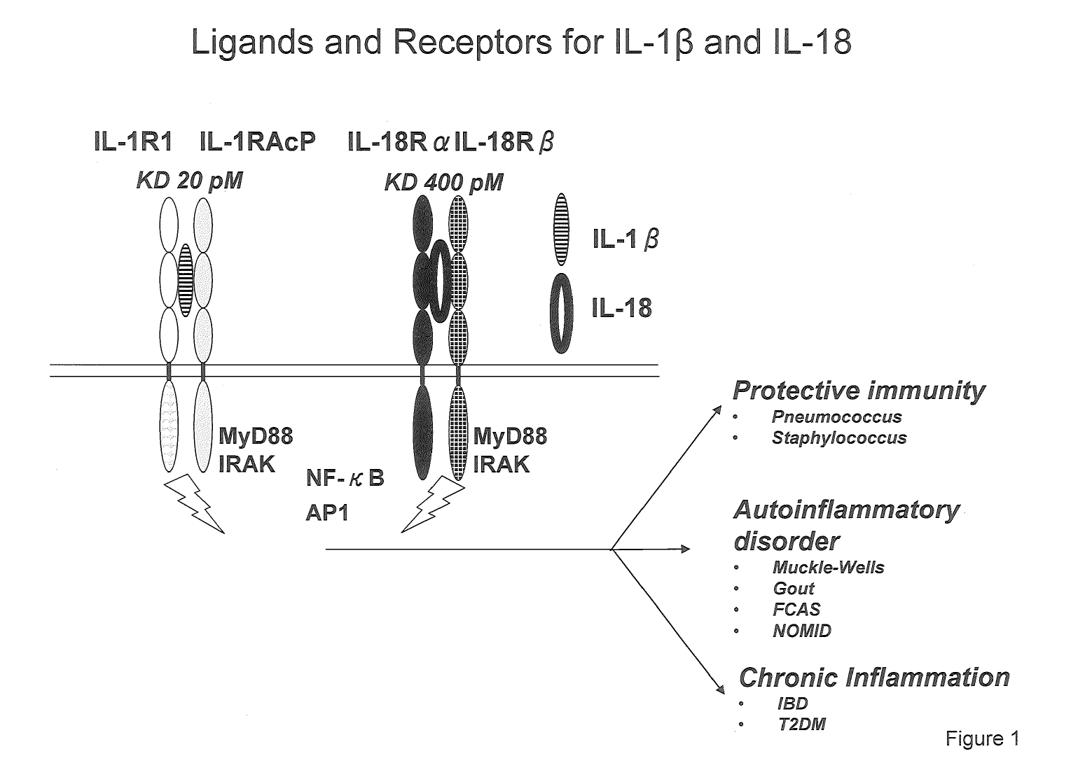

FIG. 2 depicts an example of a hypothetical model for IL-1β/IL-18 involvement in inflammatory bowel disease. Stimulation of lamina propria macrophages by intestinal microbes leads to autocatalytic activation of caspase-1 that in turn processes and secretes IL-1β and IL-18. IL-1β and IL-18 act on various immune cells and induce pro-inflammatory cytokines in macrophages, polarize T-cells towards Th1 and Th17 pathogenic T cells and disrupt the epithelial barrier, enabling more pathogens to stimulate macrophages.

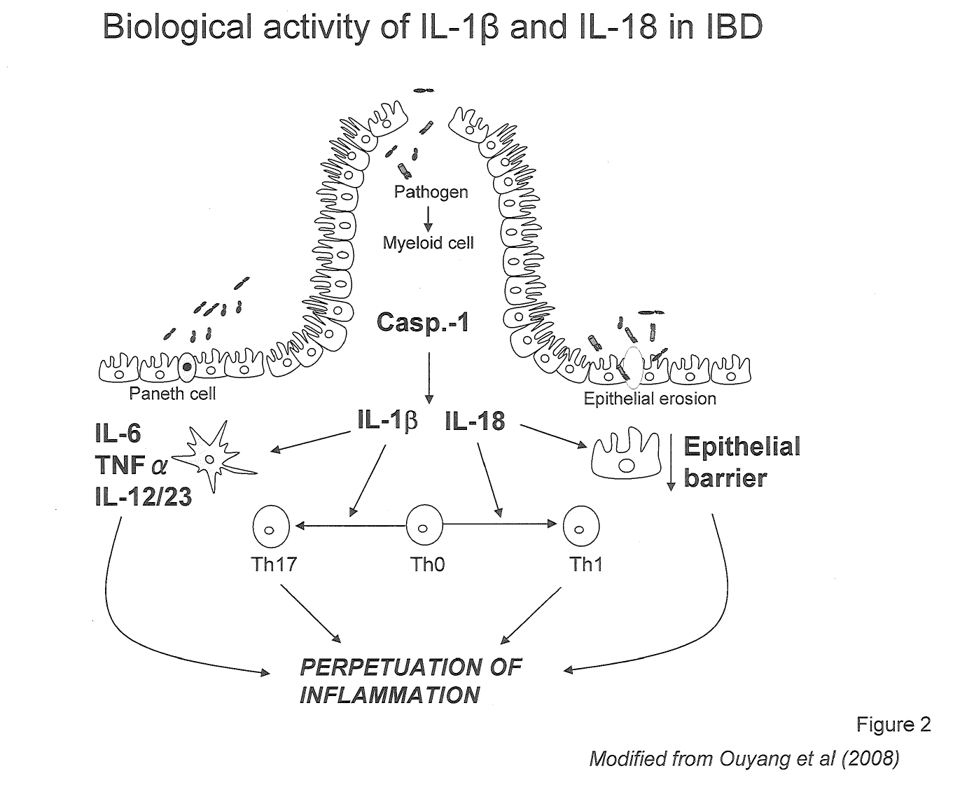

FIG. 3 depicts an example of how genetics suggest a role for inflammasome activation in Crohn's disease. Polymophisms in autophagy-related genes ATG16L1 and IRGM and inflammasome regulating genes NOD2 and NALP3 results in increased Caspase-1 activation and secretion of IL-1β and IL-18.

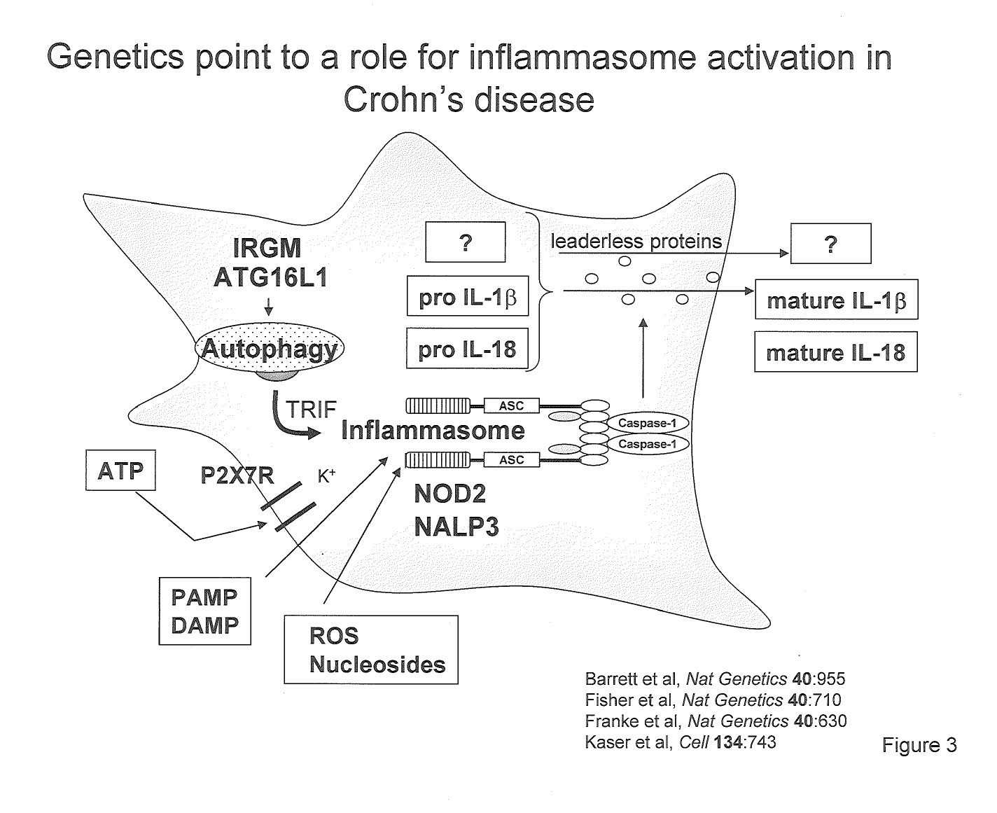

FIG. 4 (A) is data showing that expression of IL-1β and IL-18 mRNA is increased in colon biopsies from Crohn's and UC patients. The values are based on relative intensities of the hybridization signal on an Agilent gene platform. (B) is data showing that IL-1β and IL-18 are increased in serum from patients with Crohn's disease and UC.

FIG. 5 is data showing that differential expression of IL-1β and IL-18 in inflamed colon. Immunohistochemistry on cross-sections through colon biopsies from patients with UC. Sections were stained with antibody to human IL-1β and IL-18. While IL-1β is primarily found in macrophages present at sites of transmural inflammation, IL-18 is predominantly found in dendritic cells present in lymphoid follicles. In both cases, staining was only observed in regions of inflammation.

FIG. 6 is data showing that increased secretion of IL-1β and IL-18 from colons of mice receiving 3.5% DSS in their drinking water ad libitum for 5 days

FIG. 7 is data showing that increased secretion of IL-1β and IL-18 from colons of mice receiving adoptively transferred CD4+CD45RBhi T cells.

FIG. 8 is data showing that increased secretion of IL-1β and IL-18 from colons of IL-10 KO mice treated with piroxicam.

FIG. 9 is data showing that that IL-1R1 and ASC KO mice show significantly reduced severity of DSS-induced colitis. Colon scores from mice deficient in IL-1R1, IL-18Ra and ASC.

FIG. 10 IL-1R1 deficiency leads to a significant reduction of IL-1β, IL-18, IL-17 and TNF-alpha in DSS-induced colitis.

FIG. 11 is data showing that IL-18R deficiency leads to a significant reduction in the levels of IL-1β and IL-12p40 in DSS-induced colitis.

FIG. 12 is data showing that ASC deficiency leads to a significant reduction in the levels of IL-1β, IL-18, IL-12p40 and IL-17 in DSS-induced colitis.

FIG. 13 is a summary of exemplary cytokine responses in ex-vivo colon cultures obtained from various mouse IBD models.

FIG. 14 is data showing that that IL-1β is expressed in vitreous of a subpopulation of AMD patients. Vitreous was collected from patients diagnosed with wet AMD, geographic atrophy (GA) or from patients with a macular pucker or macular hole. Cytokine levels were determined using and ELISA assay.

FIG. 15 is data showing increased IL-1β and Caspase-1 expression in the eye following constant light exposure. In (A) Mice were exposed to constant light (1800 Lux) for 10 days, after which eyes were removed; in (B) mRNA was isolated from the retina, and IL-13 mRNA levels were determined by real-time PCR; and in (C) Whole eyes were homogenized in lysis buffer, and cell extracts were separated on an SDS gel, blotted and stained with an antibody to murine caspase-1.

FIG. 16 is data showing expression of pro-IL-1β and caspase-1 in IL-1β-infected eyes. Adeno-associated virus (AAV) expressing mature murine IL-1β was injected sub-retinally. Three weeks later, mice were exposed to intense light (5000 Lux) (ILE; intense light exposure) for 6 hrs. Eyes were processed 1 day later for Western blot analysis of IL-1β and Caspase-1 as described in FIG. 15.

FIG. 17 is data showing increased inflammation and neo-angiogenesis following IL1β over-expression in the mouse eye. In (A), albino mice received a sub-retinal injection of empty AAV virus or virus expressing IL-1β. Three weeks later, mice were injected with FITC solution and their eyes were scanned by fluorescein angiography. Arrow points to an area with choroidal neovascularization (CNV). In (B), the eyes were enucleated, fixed and processed for paraffin embedding and sectioning. Sections were stained with an antibody to CD45 to visualize infiltrating immune cells (see inset). Inflammation was absent in mice sub-retinally injected with an empty AAV vector.

FIG. 18 is data showing AAV eyes infected with pro-IL-1β show inflammation independent of caspase-1 activity. Caspase-1 wt or ko mice were injected subretinally with AAV-pro-IL-1β as described for FIG. 17. Three weeks later, the eyes were enucleated and processed as described. Inflammation proceeded independent of caspase-1 activity.

FIG. 19 is data showing that both AAV-IL1β and AAV-IL-18 significantly reduce scotopic ERG responses. Electro Retino Grams (ERGs) of mice treated with AAV-IL-1β and AAV-IL18 show significant reduction in “a” and “b”-wave responses compared to mice injected with empty vector.

FIG. 20 is a summary of the biology of IL-1β and IL-18 in AMD useful for developing anti-IL-1β and anti-IL-18 neutralizing antibodies for use in preclinical studies in nonhuman animals (e.g., mice), and as a clinical reagent for human studies.

FIG. 21 is data showing an example of a method for screening of anti-IL-1β neutralizing antibodies using an ELISA-based approach.

FIG. 22 is an ELISA assay showing the neutralizing activity of a subset of hamster anti-mouse IL-1β hybridomas.

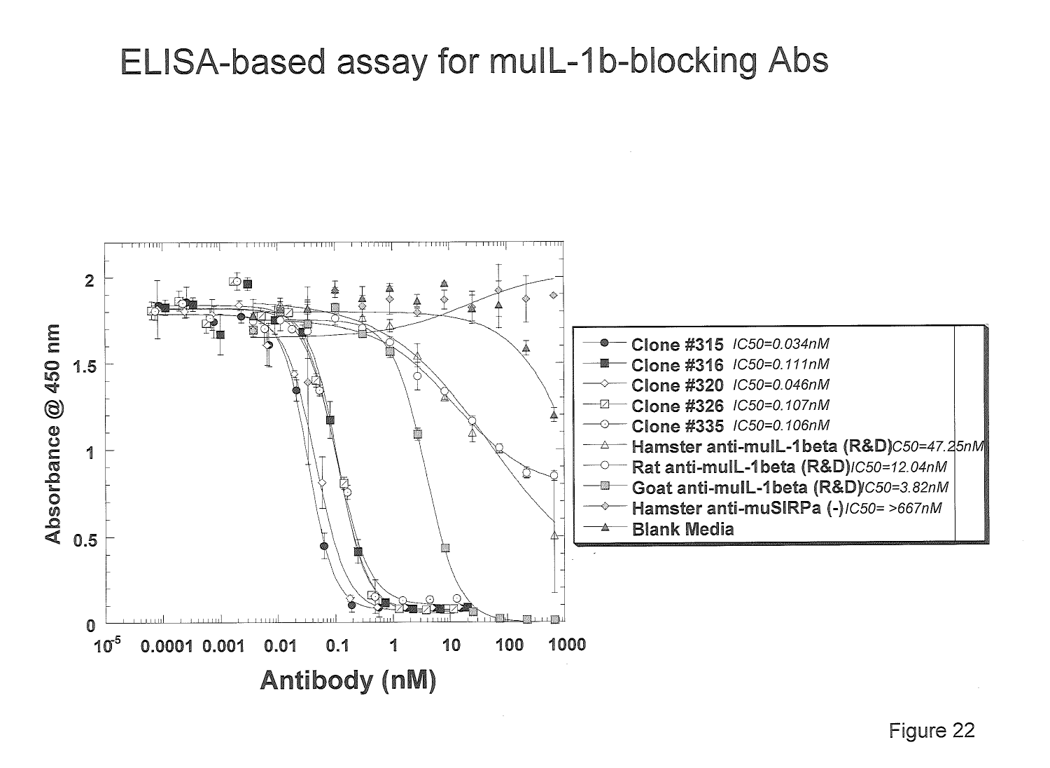

FIG. 23 is a Table illustrating the IC50 values for the blocking activity of hamster anti-mouse anti-IL-1 antibodies.

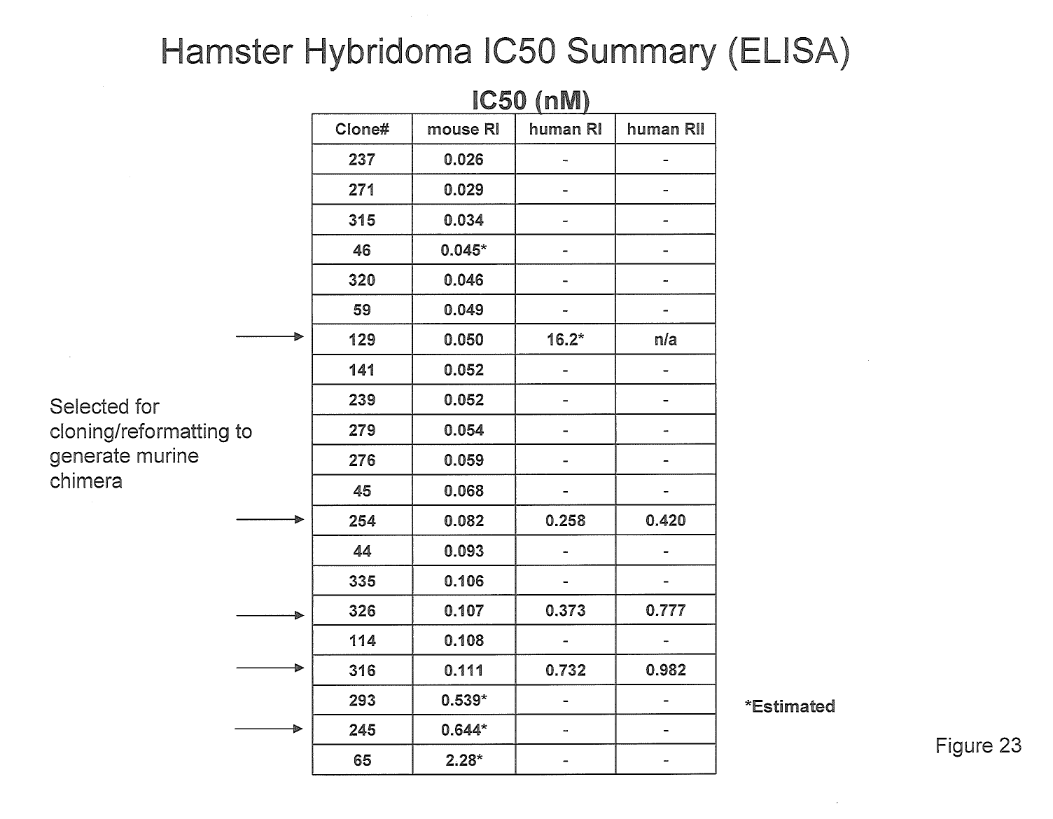

FIG. 24 is data showing cell lines used to determine neutralizing activity of human and murine IL-1β/IL-18.

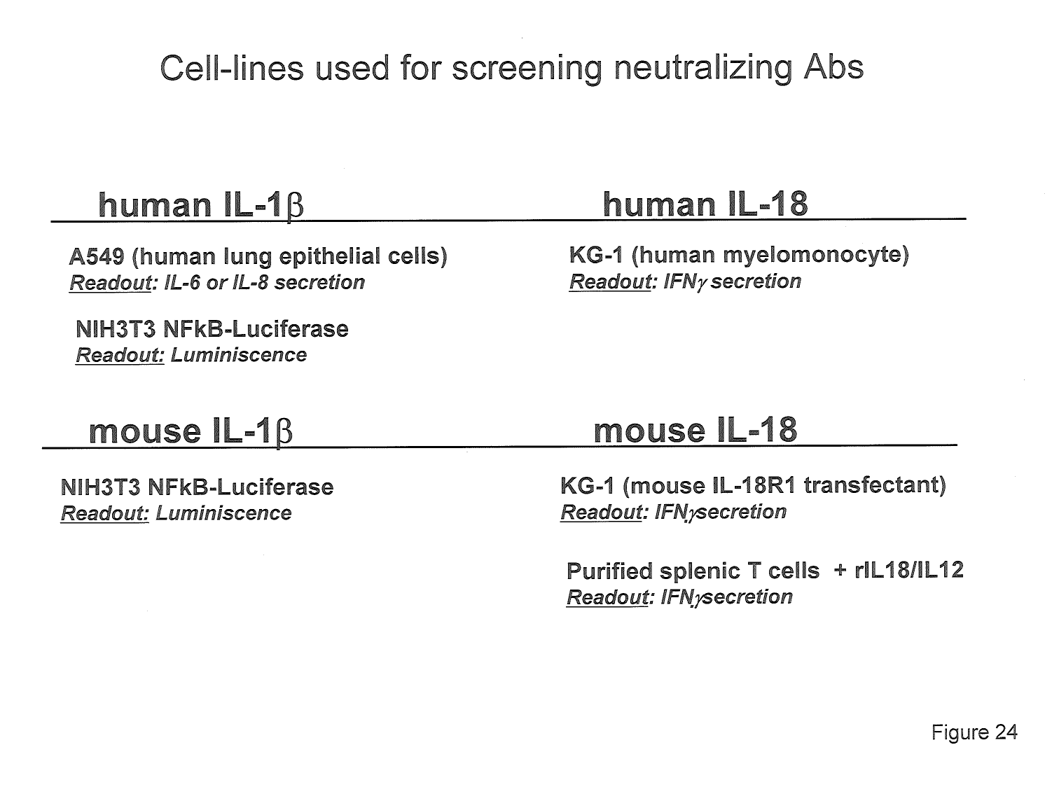

FIG. 25 is data showing (A) the dose-response of NF-kB reporter activity in an NIH3T3 cell line treated with increasing concentrations of murine IL-1β; and (B) blocking activity of hybridoma supernatants containing IL-1β-neutralizing Abs.

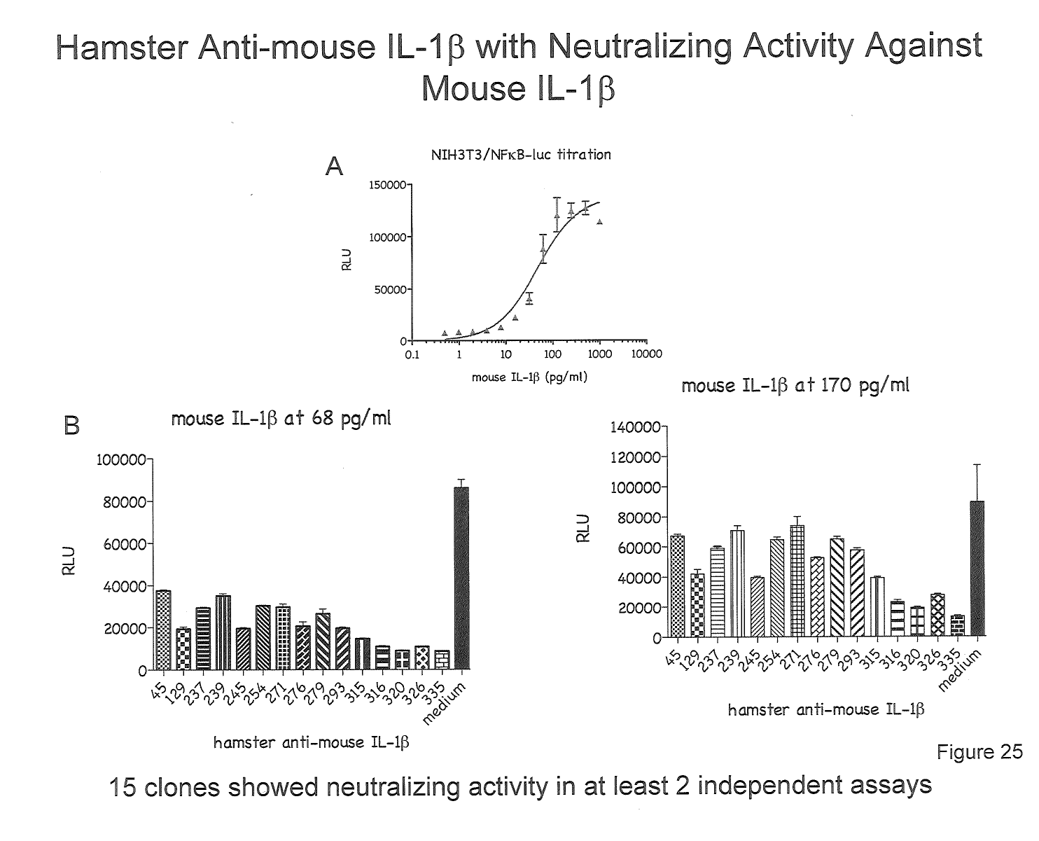

FIG. 26 is a summary of exemplary antibodies derived by phage technology. Various phage display libraries with diversity in the heavy chain variable region (V_{H}) or both the heavy and light chain variable regions (V_{H}V_{L}) were screened. Also screened was a synthetic library (YSGX) which is a reduced genetic codon library which generates randomized CDRs using codons enriched in tyrosines, serines and glycines (Fellouse et al., J Mol Biol, 373, 924-940) and a peptide library which is an antibody library designed to potentially bind specific peptide sequences.

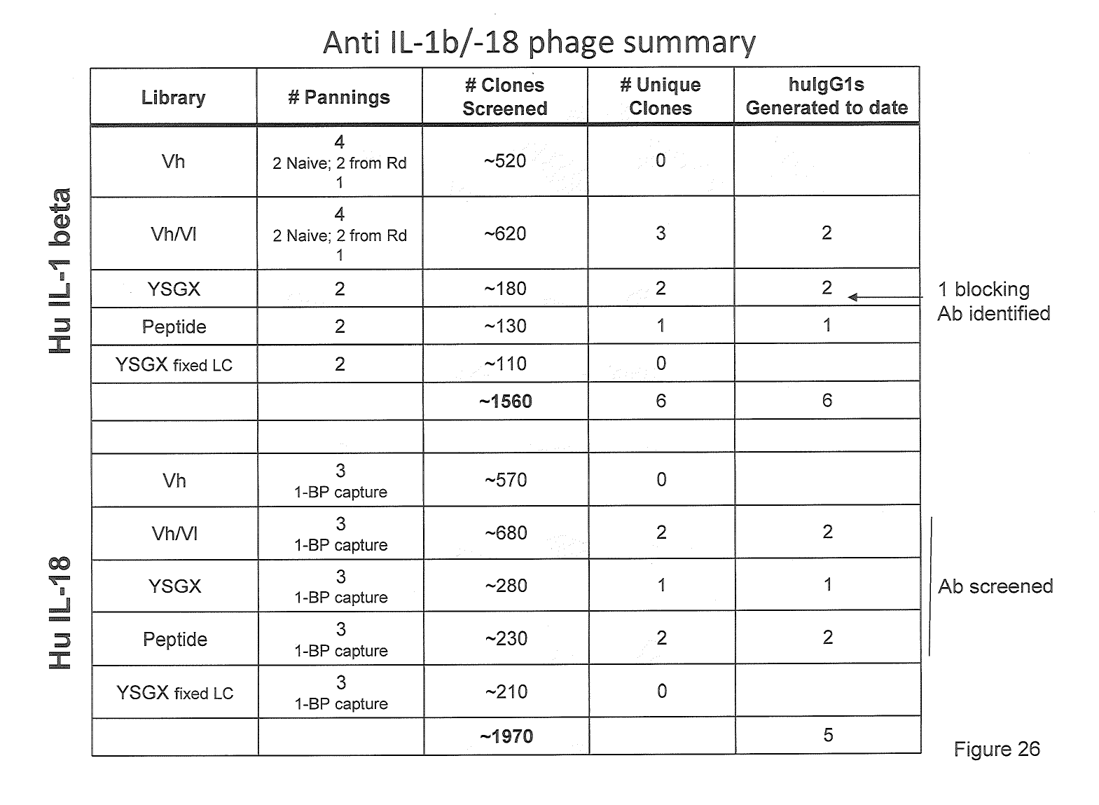

FIG. 27 is data showing the locking activity of various phage antibodies in an ELISA-based neutralization assay.

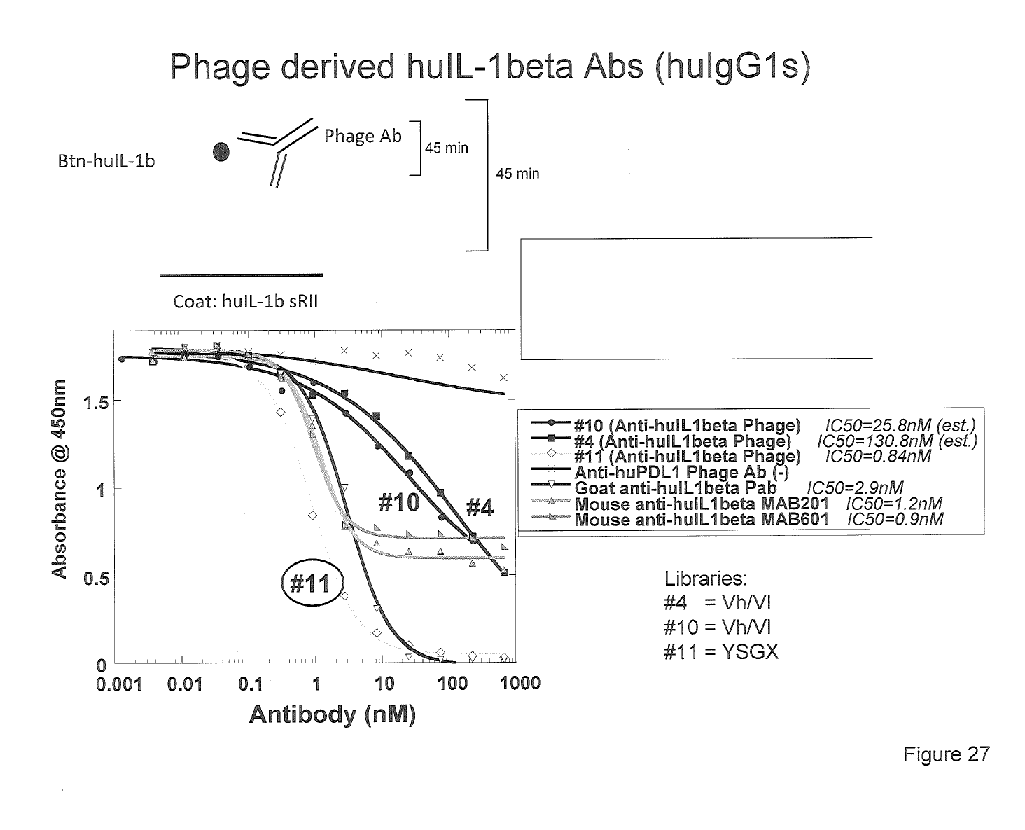

FIG. 28 is a cartoon depicting an exemplary sequence of events leading up to pancreatic beta cell loss and the potential level of intervention with anti-IL-1β/IL-18 neutralizing antibodies.

FIG. 29 is a schematic of the experimental protocol used in Example 4.

FIG. 30 is a graphic displaying the (A) histology colon score results and (B) visual colon score results of anti-IL-1β and/or anti-IL18 treatment in the piroxicam IL-10KO mouse IBD model. Also shown is the result of TNF-alpha blockade. The anti-IL-1β and anti-IL18 combination treatment was equally effective as TNF blockade.

### DETAILED DESCRIPTION OF THE INVENTION

The present invention provides anti-IL-1β and anti-IL-18 antibodies, including e.g., anti-IL-1β and anti-IL-18 bispecific antibodies, and methods of using such antibodies for treatment of disease. In some embodiments, the anti-IL-1β and anti-IL-18 antibodies are monoclonal antibodies, and are administered concurrently or consecutively to a patient, for treatment of disease. In other embodiments, the anti-IL-1β and anti-IL-18 are bispecific antibodies and are administered to a patient for treatment of disease. Examples of diseases include immune diseases and autoimmune diseases, and include inflammatory bowel disease (IBD), age-related macular degeneration (AMD), and type 2 diabetes (T2D).

In some embodiments, the anti-IL-1β and anti-IL-18 antibodies of the present invention, block or neutralize the activity of, and/or bind to, IL-1β and/or IL-18.

All references, including patents, applications, and scientific literature, cited herein are hereby incorporated by reference in their entirety.

### General Techniques

The techniques and procedures described or referenced herein are generally well understood and commonly employed using conventional methodology by those skilled in the art, such as, for example, the widely utilized methodologies described in Sambrook et al., Molecular Cloning: A Laboratory Manual 3rd. edition (2001) Cold Spring Harbor Laboratory Press, Cold Spring Harbor, N.Y.; Current Protocols in Molecular Biology (F. M. Ausubel, et al. eds., (2003)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, A Laboratory Manual, and Animal Cell Culture (R. I. Freshney, ed. (1987)); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Handbook of Experimental Immunology (D. M. Weir and C. C. Blackwell, eds.); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J. B. Lippincott Company, 1993).

### DEFINITIONS

For purposes of interpreting this specification, the following definitions will apply and whenever appropriate, terms used in the singular will also include the plural and vice versa. In the event that any definition set forth below conflicts with any document incorporated herein by reference, the definition set forth below shall control.

With reference to the molecules referred to herein (e.g., antibodies, and the molecules that the antibodies bind to), “active” or “activity” refer to biological, immunological and/or functional activities of such molecules. For example, in some embodiments, the anti-IL-1β and anti-IL-18 antibodies of the present invention are bispecific antibodies that bind to IL-1β and IL-18 and thus have a binding activity. In a further embodiment, anti-IL-1β and anti-IL-18 antibodies of the present invention have neutralizing or blocking activity, i.e., such antibodies can neutralize or block the activity of IL-1β and/or IL-18.

“Affibodies” or “Affibody” refers to the use of a protein liked by peptide bond to an Fc region, wherein the protein is used as a scaffold to provide a binding surface for a target molecule. The binding surface may be altered through mutagenisis to generate a library of proteins that can bind other target molecules or other epitopes on the same target molecule. The starting protein is often a naturally occurring protein such as staphylococcal protein A or IgG-binding B domain, or the Z protein derived therefrom (see Nilsson et al (1987), Prot Eng 1, 107-133, and U.S. Pat. No. 5,143,844) or a fragment or derivative thereof. For example, affibodies can be created from Z proteins variants having altered binding affinity to target molecule(s), wherein a segment of the Z protein has been mutated by random mutagenesis to create a library of variants capable of binding a target molecule. Examples of affibodies include U.S. Pat. No. 6,534,628, Nord K et al, Prot Eng 8:601-608 (1995) and Nord K et al, Nat Biotech 15:772-777 (1997). Biotechnol Appl Biochem. 2008 June; 50(Pt 2):97-112.

The term “antibody” herein is used in the broadest sense and refers to any immunoglobulin (Ig) molecule whether naturally occurring or engineered, and any fragment, mutant, variant or derivation thereof which so long as it exhibits the desired biological activity (e.g., epitope binding activity). Examples of antibodies include, but are not limited to, monoclonal antibodies, polyclonal antibodies, multispecific antibodies, antibody fragments, single domain antibodies, octopus antibodies and DVD antibodies. In one embodiment, an antibody of the present invention comprises at least one variable domain. In another embodiment, an antibody of the present invention is a bispecific antibody.

Generally, immunoglobulins are assigned to different classes, depending on the amino acid sequences of the heavy chain constant domains. Five major classes of immunoglobulins have been described: IgA, IgD, IgE, IgG and IgM. These may be further divided into subclasses (isotypes), e.g., IgG-1, IgG-2, IgA-1, IgA-2, and the like. The heavy chain constant domains corresponding to each immunoglobulin class are termed α, δ, ε, γ and μ for IgA, D, E, G, and M, respectively. The subunit structures and three-dimensional configurations of the different classes of immunoglobulins are well known and described generally, for example, in Abbas et al., 2000, Cellular and Mol. Immunology, 4th ed. An antibody may be part of a larger fusion molecule, formed by covalent or non-covalent association of the antibody with one or more other protein or peptide.

In one embodiment, antibodies of the present invention have reduced (fewer) disulfide linkages. In one embodiment, antibodies of the invention comprise a hinge region in which at least one cysteine residue is rendered incapable of forming a disulfide linkage, wherein the disulfide linkage is preferably intermolecular, preferably between two heavy chains. A hinge cysteine can be rendered incapable of forming a disulfide linkage by any of a variety of suitable methods known in the art, some of which are described herein, including but not limited to deletion of the cysteine residue or substitution of the cysteine with another amino acid.

The phrase “an anti-IL-1β antibody and/or anti-IL-18 antibody/antibodies” refers, depending on the context, to (1) an anti-IL-1β antibody, or (2) an anti-IL-18 antibody, or (3) a combination of an anti-IL-1β antibody and an anti-IL-18 antibody (i.e., two antibodies), or (4) an antibody that binds to both IL-1β and IL-18.

An “affinity matured” antibody is one having one or more alteration in one or more CDRs thereof which result in an improvement in the affinity of the antibody for antigen, compared to a parent antibody which does not possess those alteration(s). Preferred affinity matured antibodies will have nanomolar or even picomolar affinities for the target antigen. Affinity matured antibodies are produced by known procedures. See, for example, Marks et al., 1992, Biotechnology 10:779-783 that describes affinity maturation by variable heavy chain (V_{H}) and variable light chain (V_{L}) domain shuffling. Random mutagenesis of CDR and/or framework residues is described in: Barbas, et al. 1994, Proc. Nat. Acad. Sci, USA 91:3809-3813; Shier et al., 1995, Gene 169:147-155; Yelton et al., 1995, J. Immunol. 155:1994-2004; Jackson et al., 1995, J. Immunol. 154(7):3310-9; and Hawkins et al, 1992, J. Mol. Biol. 226:889-896, for example.

An “agonist antibody” or “agonistic antibody” is an antibody that binds and activates an antigen, such as a receptor. Generally, receptor activation capability of the agonist antibody will be at least qualitatively similar (and may be essentially quantitatively similar) to that of a native agonist ligand of the receptor.

“Antibody fragments” refers to an antibody comprising a portion of an intact antibody, preferably the antigen binding or a variable region of the intact antibody. Examples of antibody fragments include Fab, Fab′, F(ab)₂, and Fv fragments; diabodies (Db); tandem diabodies (taDb), linear antibodies (see U.S. Pat. No. 5,641,870, Example 2; Zapata et al., Protein Eng. 8(10):1057-1062 (1995)); one-armed antibodies, minibodies, single-chain antibody molecules; and multispecific antibodies formed from antibody fragments (e.g., including but not limited to, Db-Fc, taDb-Fc, taDb-CH3 and (scFV)4-Fc).

In some embodiments, an antibody fragment comprises only a portion of an intact antibody, where the portion retains at least one, and may retain most or all, of the functions normally associated with that portion when present in an intact antibody. In another embody, an antibody fragment of the invention comprises a sufficient portion of the constant region to permit dimerization (or multimerization) of heavy chains that have reduced disulfide linkage capability, for example where at least one of the hinge cysteines normally involved in inter-heavy chain disulfide linkage is altered as described herein. In one embodiment, an antibody fragment comprises an antigen binding site or variable domains of the intact antibody and thus retains the ability to bind antigen. In another embodiment, an antibody fragment, for example one that comprises the Fc region, retains at least one of the biological functions normally associated with the Fc region when present in an intact antibody, such as FcRn binding, antibody half life modulation, ADCC function, and/or complement binding (for example, where the antibody has a glycosylation profile necessary for ADCC function or complement binding). Examples of antibody fragments include, but are not limited to, linear antibodies; single-chain antibody molecules; and multispecific antibodies formed from antibody fragments.

“Antibody-dependent cell-mediated cytotoxicity” and “ADCC” refer to a cell-mediated reaction in which nonspecific cytotoxic cells that express FcRs (such as Natural Killer (NK) cells, neutrophils, and macrophages) recognize bound antibody on a target cell and subsequently cause lysis of the target cell. NK cells, the primary cells for mediating ADCC, express only FcγRIII, whereas monocytes express FcγRI, FcγRII, and FcγRIII. FcR expression on hematopoietic cells is summarized in Table 3 on page 464 of Ravetch et al., 1991, Annu. Rev. Immunol 9:457-92. To assess ADCC activity of a molecule of interest, an in vitro ADCC assay such as that described in U.S. Pat. No. 5,500,362 or 5,821,337 may be performed. Useful effector cells for such assays include peripheral blood mononuclear cells (PBMC) and Natural Killer (NK) cells. Alternatively, or additionally, ADCC activity of the molecule of interest may be assessed in vivo, for example, in a animal model such as that disclosed in Clynes et al., 1998, PNAS (USA) 95:652-656.

The terms “anti-IL-1β antibody” and “an antibody that binds to IL-1β” refer to an antibody that is capable of binding IL-1β with sufficient affinity such that the antibody is useful as a diagnostic and/or therapeutic agent in targeting IL-1β. In one embodiment, the extent of binding of an anti-IL-1β antibody to an unrelated, non-IL-1β protein is less than about 10% of the binding of the antibody to IL-1β as measured, e.g., by a radioimmunoassay (RIA). In certain embodiments, an anti-IL-1β antibody binds to an epitope of IL-1β that is conserved among IL-1β from different species.

The terms “anti-IL-18 antibody” and “an antibody that binds to IL-18” refer to an antibody that is capable of binding IL-18 with sufficient affinity such that the antibody is useful as a diagnostic and/or therapeutic agent in targeting IL-18. In one embodiment, the extent of binding of an anti-IL-18 antibody to an unrelated, non-IL-18 protein is less than about 10% of the binding of the antibody to IL-18 as measured, e.g., by a radioimmunoassay (RIA). In certain embodiments, an anti-IL-18 antibody binds to an epitope of IL-18 that is conserved among IL-18 from different species.

An “autoimmune disease” as used herein is a non-malignant disease or disorder arising from and directed against an individual's own tissues. The autoimmune diseases described herein specifically exclude malignant or cancerous diseases or conditions, particularly excluding B cell lymphoma, acute lymphoblastic leukemia (ALL), chronic lymphocytic leukemia (CLL), Hairy cell leukemia, and chronic myeloblastic leukemia. Examples of autoimmune diseases or disorders include, but are not limited to, age-related macular degeneration (AMD), inflammatory responses such as inflammatory skin diseases including psoriasis and dermatitis (for example, atopic dermatitis); systemic scleroderma and sclerosis; responses associated with inflammatory bowel disease (such as Crohn's disease and ulcerative colitis); respiratory distress syndrome (including adult respiratory distress syndrome; ARDS); dermatitis; meningitis; encephalitis; uveitis; colitis; glomerulonephritis; allergic conditions such as eczema and asthma and other conditions involving infiltration of T cells and chronic inflammatory responses; atherosclerosis; leukocyte adhesion deficiency; rheumatoid arthritis; systemic lupus erythematosus (SLE); lupus nephritis (LN); diabetes mellitus (e.g. Type I diabetes mellitus or insulin dependent diabetes mellitis); multiple sclerosis; Reynaud's syndrome; autoimmune thyroiditis; allergic encephalomyelitis; Sjorgen's syndrome; juvenile onset diabetes; and immune responses associated with acute and delayed hypersensitivity mediated by cytokines and T-lymphocytes typically found in tuberculosis, sarcoidosis, polymyositis, granulomatosis and vasculitis; pernicious anemia (Addison's disease); diseases involving leukocyte diapedesis; central nervous system (CNS) inflammatory disorder; multiple organ injury syndrome; hemolytic anemia (including, but not limited to cryoglobinemia or Coombs positive anemia); myasthenia gravis; antigen-antibody complex mediated diseases; anti-glomerular basement membrane disease; antiphospholipid syndrome; allergic neuritis; Graves' disease; Lambert-Eaton myasthenic syndrome; pemphigoid bullous; pemphigus; autoimmune polyendocrinopathies; Reiter's disease; stiff-man syndrome; Behcet disease; giant cell arteritis; immune complex nephritis; IgA nephropathy; IgM polyneuropathies; immune thrombocytopenic purpura (ITP) or autoimmune thrombocytopenia etc.

AMD is a leading cause of severe, irreversible vision loss among the elderly (see e.g., Bressler (2004) JAMA 291:1900-01). It is characterized by a broad spectrum of clinical and pathologic findings, including pale yellow spots known as drusen, disruption of the retinal pigment epithelium (RPE), choroidal neovascularization (CNV), and disciform macular degeneration. The manifestations of the disease is classified into two forms: non-exudative (dry) and exudative (wet or neovascular). Recently, several therapies for treatment of wet AMD have been approved—photodynamic therapy using verteporfin (Visudyne®); a VEGF-binding aptamer, pegaptantib (Macugen®); and an anti-VEGF antibody fragment, ranibizumab (Lucentis®).

An “autoinflammatory disease” as used herein refers to a group of rare hereditary immune-mediated disorders that share similar features, particularly fever. Autoinflammatory diseases are characterized by recurrent unprovoked inflammation in the absence of high titers of autoantibodies, infection, or antigen-specific T lymphocytes. Exemplary autoinflammatory diseases include, but is not limited to, Familial Mediterranean Fever (FMF); tumour necrosis factor (TNF) receptor-associated periodic fever syndrome (TRAPS); hyperimmunoglobulinemia D and periodic fever syndrome (HIDS); systemic onset juvenile idiopathic arthritis (Still's disease); cryopyrin-associated periodic syndrome (CAPS); familial cold autoinflammatory syndrome; Muckle-Wells syndrome; deficiency of the interleukin-1 receptor antagonist (DIRA); and neonatal onset multi-system inflammatory disease (NOMID)/chronic infantile neurological cutaneous and articular (CINCA) syndrome.

Autoimmune and autoinframmatory diseases share common characteristics in that both groups of disorders result from the immune system attacking the body's own tissues; and also result in increased inflammation. The distinguishing feature between the two is the lack of autoantibodies (at high titers) in autoinflammatory diseases.

A “biologically active” or “functional” immunoglobulin is one capable of exerting one or more of its natural activities in structural, regulatory, biochemical or biophysical events. For example, a biologically active antibody may have the ability to specifically bind an antigen and the binding may elicit or alter a cellular or molecular event such as signaling transduction or enzymatic activity. A biologically active antibody may also block ligand activation of a receptor or act as an agonist antibody. The capability of an antibody to exert one or more of its natural activities depends on several factors, including proper folding and assembly of the polypeptide chains.

“Binding affinity” generally refers to the strength of the sum total of noncovalent interactions between a single binding site of a molecule (e.g., an antibody) and its binding partner (e.g., an antigen). The affinity of a molecule X for its partner Y can generally be represented by the dissociation constant (Kd). Affinity can be measured by common methods known in the art, including those described herein. Low-affinity antibodies bind antigen weakly and tend to dissociate readily, whereas high-affinity antibodies bind antigen more tightly and remain bound longer.

“Biological molecule” refers to a nucleic acid, a protein, a carbohydrate, a lipid, and combinations thereof. In one embodiment, the biologic molecule exists in nature.

A “blocking” antibody or an “antagonist” antibody is one that inhibits or reduces biological activity of the antigen it binds. Such blocking can occur by any means, for example, by interfering with: ligand binding to the receptor, receptor complex formation, tyrosine kinase activity of a tyrosine kinase receptor in a receptor complex and/or phosphorylation of tyrosine kinase residue(s) in or by the receptor. Preferred blocking antibodies or antagonist antibodies substantially or completely inhibit the biological activity of the antigen.

The terms “cancer” and “cancerous” refer to or describe the physiological condition in mammals that is typically characterized by unregulated cell growth. Examples of cancer include but are not limited to, carcinoma, lymphoma, blastoma, sarcoma, and leukemia. More particular examples of such cancers include squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, prostate cancer, vulval cancer, thyroid cancer, hepatic carcinoma, and various types of head and neck cancer.

The term “chimeric” antibodies refer to antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (See, for example, U.S. Pat. No. 4,816,567 and Morrison et al., 1984, Proc. Natl. Acad. Sci. USA 81:6851-6855).

As used herein, the expressions “cell,” “cell line,” and “cell culture” are used interchangeably and all such designations include progeny. Thus, the words “transformants” and “transformed cells” include the primary subject cell and cultures derived therefrom without regard for the number of transfers. It is also understood that all progeny may not be precisely identical in DNA content, due to deliberate or inadvertent mutations. Mutant progeny that have the same function or biological activity as screened for in the originally transformed cell are included. Where distinct designations are intended, it will be clear from the context.

The expression “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

“Diabetes” as used herein is a chronic disorder affecting carbohydrate, fat and protein metabolism in animals. Diabetes is the leading cause of blindness, renal failure, and lower limb amputations in adults and is a major risk factor for cardiovascular disease and stroke. Type I diabetes mellitus (or insulin-dependent diabetes mellitus (“IDDM”) or juvenile-onset diabetes) comprises approximately 10% of all diabetes cases. The disease is characterized by a progressive loss of insulin secretory function by beta cells of the pancreas. This characteristic is also shared by non-idiopathic, or “secondary”, diabetes having its origins in pancreatic disease. Type I diabetes mellitus is associated with the following clinical signs or symptoms, e.g., persistently elevated plasma glucose concentration or hyperglycemia; polyuria; polydipsia and/or hyperphagia; chronic microvascular complications such as retinopathy, nephropathy and neuropathy; and macrovascular complications such as hyperlipidemia and hypertension which can lead to blindness, end-stage renal disease, limb amputation and myocardial infarction.

Type II diabetes mellitus (non-insulin-dependent diabetes mellitus or NIDDM) is a metabolic disorder involving the dysregulation of glucose metabolism and impaired insulin sensitivity. Type II diabetes mellitus usually develops in adulthood and is associated with the body's inability to utilize or make sufficient insulin. In addition to the insulin resistance observed in the target tissues, patients suffering from type II diabetes mellitus have a relative insulin deficiency—that is, patients have lower than predicted insulin levels for a given plasma glucose concentration. Type II diabetes mellitus is characterized by the following clinical signs or symptoms, e.g., persistently elevated plasma glucose concentration or hyperglycemia; polyuria; polydipsia and/or hyperphagia; chronic microvascular complications such as retinopathy, nephropathy and neuropathy; and macrovascular complications such as hyperlipidemia and hypertension which can lead to blindness, end-stage renal disease, limb amputation and myocardial infarction. Syndrome X, also termed Insulin Resistance Syndrome (IRS), Metabolic Syndrome, or Metabolic Syndrome X, is recognized in some 2% of diagnostic coronary catheterizations. Often disabling, it presents symptoms or risk factors for the development of Type II diabetes mellitus and cardiovascular disease, including, e.g., impaired glucose tolerance (IGT), impaired fasting glucose (IFG), hyperinsulinemia, insulin resistance, dyslipidemia (e.g., high triglycerides, low HDL), hypertension and obesity.

A “disorder” is any condition that would benefit from treatment with a therapeutic antibody or antibodies. This includes chronic and acute disorders or diseases including those pathological conditions which predispose the mammal to the disorder in question. In some embodiments, the disorder is a cancer, an inflammatory, an immune, an autoinflammatory or an autoimmune disease.

An “extracellular domain” is defined herein as that region of a transmembrane polypeptide, such as an FcR, that is external to a cell.

The terms “Fc receptor” or “FcR” are used to describe a receptor that binds to the Fc region of an antibody.

The term “Fc region” herein is used to define a C-terminal region of an immunoglobulin heavy chain, including native sequence Fc regions and variant Fc regions. In one embodiment, the Fc region comprises a CH2 domain and/or a CH3 domain. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue. “Fc complex” as used herein refers to two CH2 domains and/or two CH3 domains, wherein the CH2 domains and/or the CH3 domains are bound together through interactions that are not peptide bonds.

“Framework regions” (FR) are those variable domain residues other than the CDR residues. Each IgG variable domain typically has four FRs identified as FR1, FR2, FR3, and FR4. If the CDRs are defined according to Kabat, the light chain FR residues are positioned at about residues 1-23 (LCFR1), 35-49 (LCFR2), 57-88 (LCFR3), and 98-107 (LCFR4) and the heavy chain FR residues are positioned about at residues 1-30 (HCFR1), 36-49 (HCFR2), 66-94 (HCFR3), and 103-113 (HCFR4) in the heavy chain residues. If the CDRs comprise amino acid residues from hypervariable loops, the light chain FR residues are positioned about at residues 1-25 (LCFR1), 33-49 (LCFR2), 53-90 (LCFR3), and 97-107 (LCFR4) in the light chain and the heavy chain FR residues are positioned about at residues 1-25 (HCFR1), 33-52 (HCFR2), 56-95 (HCFR3), and 102-113 (HCFR4) in the heavy chain residues. In some instances, when the CDR comprises amino acids from both a CDR as defined by Kabat and those of a hypervariable loop, the FR residues will be adjusted accordingly. For example, when CDRH1 includes amino acids H26-H35, the heavy chain FR1 residues are at positions 1-25 and the FR2 residues are at positions 36-49.

A “functional Fc region” possesses an “effector function” of a native sequence Fc region. Exemplary “effector functions” include C1q binding; complement dependent cytotoxicity; Fc receptor binding; antibody-dependent cell-mediated cytotoxicity (ADCC); phagocytosis; down regulation of cell surface receptors (e.g. B cell receptor; BCR), and the like. Such effector functions generally require the Fc region to be combined with a binding domain (e.g. an antibody variable domain) and can be assessed using various assays as, for example, those disclosed herein. A “native sequence Fc region” comprises an amino acid sequence identical to the amino acid sequence of a Fc region found in nature. Native sequence human Fc regions include a native sequence human IgG1 Fc region (non-A and A allotypes); native sequence human IgG2 Fc region; native sequence human IgG3 Fc region; and native sequence human IgG4 Fc region as well as naturally occurring variants thereof. A “variant Fc region” comprises an amino acid sequence that differs from a native sequence Fc region by virtue of at least one “amino acid modification” as herein defined. The variant Fc region can have at least one amino acid substitution compared to a native sequence Fc region or to the Fc region of a parent antibody, and may have, for example, from about one to about ten amino acid substitutions, or from about one to about five amino acid substitutions in a native sequence Fc region or in the Fc region of the parent antibody. The variant Fc region can possess at least about 80% identity with a native sequence Fc region and/or with an Fc region of a parent antibody, and may have at least about 90% identity therewith, or have at least about 95% identity therewith.

The terms “full length antibody,” “intact antibody” and “whole antibody” are used herein interchangeably, to refer to an antibody in its substantially intact form, and not antibody fragments as defined below. The terms particularly refer to an antibody with heavy chains and Fc regions. An antibody variant of the invention can be for example a full length antibody. Also, a full length antibody can be for example human, humanized, chimeric, and/or affinity matured.

A “hinge region,” and variations thereof, as used herein, includes the meaning known in the art, which is illustrated in, for example, Janeway et al., 1999, Immuno Biology: The Immune System in Health and Disease, Elsevier Science Ltd., NY. 4th ed.; Bloom et al., 1997, Protein Science, 6:407-415; Humphreys et al., 1997, J. Immunol. Methods, 209:193-202.

“Homology” is defined as the percentage of residues in the amino acid sequence variant that are identical after aligning the sequences and introducing gaps, if necessary, to achieve the maximum percent homology. Methods and computer programs for the alignment are well known in the art. One such computer program is “Align 2,” authored by Genentech, Inc., and filed with user documentation in the United States Copyright Office, Washington, D.C. 20559, on Dec. 10, 1991.

The term “host cell” (or “recombinant host cell”), as used herein, refers to a cell that has been genetically altered, or is capable of being genetically altered, by introduction of an exogenous polynucleotide, such as a recombinant plasmid or vector. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein.

A “human consensus framework” is a framework that represents the most commonly occurring amino acid residues in a selection of human immunoglobulin V_{L} or V_{H} framework sequences. Generally, the selection of human immunoglobulin V_{L} or V_{H} sequences is from a subgroup of variable domain sequences. Generally, the subgroup of sequences is a subgroup as in Kabat. In one embodiment, for the V_{L}, the subgroup is subgroup kappa I as in Kabat. In one embodiment, for the V_{H}, the subgroup is subgroup III as in Kabat.

The Kabat numbering system is generally used when referring to a residue in the variable domain (approximately residues 1-107 of the light chain and residues 1-113 of the heavy chain) (e.g., Kabat et al., Sequences of Immunological Interest. 5th Ed. Public Health Service, National Institutes of Health, Bethesda, Md. (1991)). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human IgG1 EU antibody. Unless stated otherwise herein, references to residue numbers in the variable domain of antibodies means residue numbering by the Kabat numbering system. Unless stated otherwise herein, references to residue numbers in the constant domain of antibodies means residue numbering by the EU numbering system (e.g., see U.S. Provisional Application No. 60/640,323, Figures for EU numbering).

A naturally occurring basic 4-chain antibody unit is a heterotetrameric glycoprotein composed of two identical light (L) chains and two identical heavy (H) chains (an IgM antibody consists of 5 of the basic heterotetramer units along with an additional polypeptide called J chain, and therefore contains 10 antigen binding sites, while secreted IgA antibodies can polymerize to form polyvalent assemblages comprising 2-5 of the basic 4-chain units along with J chain). In the case of IgGs, the 4-chain unit is generally about 150,000 daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has, at the N-terminus, a variable domain (V_{H}) followed by three constant domains (C_{H}) for each of the α and γ chains and four C_{H} domains for p and E isotypes. Each L chain has, at the N-terminus, a variable domain (V_{L}) followed by a constant domain (C_{L}) at its other end. The V_{L} is aligned with the V_{H} and the C_{L} is aligned with the first constant domain of the heavy chain (C_{H}1). Particular amino acid residues are believed to form an interface between the light chain and heavy chain variable domains. The pairing of a V_{H} and V_{L} together forms a single antigen-binding site. For the structure and properties of the different classes of antibodies, see, e.g., Basic and Clinical Immunology, 8th edition, Daniel P. Stites, Abba I. Terr and Tristram G. Parslow (eds.), Appleton & Lange, Norwalk, Conn., 1994, page 71 and Chapter 6.

The L chain from any vertebrate species can be assigned to one of two clearly distinct types, called kappa and lambda, based on the amino acid sequences of their constant domains. Depending on the amino acid sequence of the constant domain of their heavy chains (C_{H}), immunoglobulins can be assigned to different classes or isotypes. There are five classes of immunoglobulins: IgA, IgD, IgE, IgG, and IgM, having heavy chains designated α, δ, γ, ε, and μ, respectively. The γ and α classes are further divided into subclasses on the basis of relatively minor differences in C_{H} sequence and function, e.g., humans express the following subclasses: IgG1, IgG2, IgG3, IgG4, IgA1, and IgA2.

“Human effector cells” are leukocytes that express one or more FcRs and perform effector functions. In some embodiments the cells express at least FcγRIII and perform ADCC effector function. Examples of human leukocytes that mediate ADCC include peripheral blood mononuclear cells (PBMC), natural killer (NK) cells, monocytes, cytotoxic T cells, and neutrophils. The effector cells may be isolated from a native source, for example, from blood or PBMCs (Peripheral blood mononuclear cells) as described herein.

“Humanized” forms of non-human (for example, murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, Fv framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized an body will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

A “human antibody” is an antibody that possesses an amino acid sequence corresponding to that of an antibody produced by a human and/or has been made using any of the techniques for making human antibodies disclosed herein. This definition specifically excludes a humanized antibody that comprises non-human antigen-binding residues.

As used herein, the term “hyperglycemic disorders” refers to all forms of diabetes and disorders resulting from insulin resistance, such as Type I and Type II diabetes, as well as severe insulin resistance, hyperinsulinemia, and hyperlipidemia, e.g., obese subjects, and insulin-resistant diabetes, such as Mendenhall's Syndrome, Werner Syndrome, leprechaunism, lipoatrophic diabetes, and other lipoatrophies. A particular hyperglycemic disorder disclosed herein is diabetes, especially Type 1 and Type II diabetes. “Diabetes” itself refers to a progressive disease of carbohydrate metabolism involving inadequate production or utilization of insulin and is characterized by hyperglycemia and glycosuria.

The term “hypervariable region,” “HVR,” or “HV,” when used herein refers to the regions of an antibody variable domain which are hypervariable in sequence and/or form structurally defined loops. Generally, antibodies comprise six HVRs; three in the V_{H} (H1, H2, H3), and three in the V_{L} (L1, L2, L3). In native antibodies, H3 and L3 display the most diversity of the six HVRs, and H3 in particular is believed to play a unique role in conferring fine specificity to antibodies. See, e.g., Xu et al., Immunity 13:37-45 (2000); Johnson and Wu, in Methods in Molecular Biology 248:1-25 (Lo, ed., Human Press, Totowa, N.J., 2003). However, there are a number of examples of naturally occurring and engineered, functional antibodies having only a heavy chain and lacking a light chain. See, e.g., Hamers-Casterman et al., Nature 363:446-448 (1993); Sheriff et al., Nature Struct. Biol. 3:733-736 (1996).

A number of HVR delineations are in use and are encompassed herein. The Kabat Complementarity Determining Regions (CDRs) are based on sequence variability and are the most commonly used (Kabat et al., Sequences of Proteins of Immunological Interest, 5th Ed. Public Health Service, National Institutes of Health, Bethesda, Md. (1991)). Chothia refers instead to the location of the structural loops (Chothia and Lesk J. Mol. Biol. 196:901-917 (1987)). The AbM HVRs represent a compromise between the Kabat HVRs and Chothia structural loops, and are used by Oxford Molecular's AbM antibody modeling software. The “contact” HVRs are based on an analysis of the available complex crystal structures. The residues from each of these HVRs are noted below.

| Loop | Kabat | AbM | Chothia | Contact |
|---|---|---|---|---|
| L1 | L24-L34 | L24-L34 | L26-L32 | L30-L36 |
| L2 | L50-L56 | L50-L56 | L50-L52 | L46-L55 |
| L3 | L89-L97 | L89-L97 | L91-L96 | L89-L96 |
| H1 (Kabat | H31-H35B | H26-H35B | H26-H32 | H30-H35B |
| Numbering) | | | | |
| H1 (Chothia | H31-H35 | H26-H35 | H26-H32 | H30-H35 |
| Numbering) | | | | |
| H2 | H50-H65 | H50-H58 | H53-H55 | H47-H58 |
| H3 | H95-H102 | H95-H102 | H96-H101 | H93-H101 |

HVRs generally comprise amino acid residues from the hypervariable loops and/or from the “complementarity determining regions” (CDRs), the latter being of highest sequence variability and/or involved in antigen recognition. Exemplary hypervariable loops occur at amino acid residues 26-32 (L1), 50-52 (L2), 91-96 (L3), 26-32 (H1), 53-55 (H2), and 96-101 (H3). (Chothia and Lesk, J. Mol. Biol. 196:901-917 (1987).) Exemplary CDRs (CDR-L1, CDR-L2, CDR-L3, CDR-H1, CDR-H2, and CDR-H3) occur at amino acid residues 24-34 of L1, 50-56 of L2, 89-97 of L3, 31-35B of H1, 50-65 of H2, and 95-102 of H3. (Kabat et al., Sequences of Proteins of Immunological Interest, 5th Ed. Public Health Service, National Institutes of Health, Bethesda, Md. (1991).) With the exception of CDR1 in V_{H}, CDRs generally comprise the amino acid residues that form the hypervariable loops. CDRs also comprise “specificity determining residues,” or “SDRs,” which are residues that contact antigen. SDRs are contained within regions of the CDRs called abbreviated-CDRs, or a-CDRs. Exemplary a-CDRs (a-CDR-L1, a-CDR-L2, a-CDR-L3, a-CDR-H1, a-CDR-H2, and a-CDR-H3) occur at amino acid residues 31-34 of L1, 50-55 of L2, 89-96 of L3, 31-35B of H1, 50-58 of H2, and 95-102 of H3. (See Almagro and Fransson, Front. Biosci. 13:1619-1633 (2008).) IgG HVRs may comprise “extended HVRs” as follows: 24-36 or 24-34 (L1), 46-56 or 50-56 (L2) and 89-97 or 89-96 (L3) in the V_{L} and 26-35 (H1), 50-65 or 49-65 (H2) and 93-102, 94-102, or 95-102 (H3) in the V_{H}. Unless otherwise indicated, HVR residues and other residues in the variable domain (e.g., FR residues) are numbered herein according to Kabat et al., supra.

Antibodies having a V_{H}/V_{L} unit that can bind two or more epitopes can be made (Bostrom et al. (2009) Science 323:1610-1614; WO 2008/027236 (incorporated by reference). Such multispecific antibodies are referred to herein as “two-in one” antibodies or “dual acting Fab” or “DAF” to indicate that a single arm of an antibody (aka the V_{H}/V_{L} unit) can bind to at least two epitopes on the same target molecule or two epitopes on different target molecules. In one aspect, these DAF antibodies can be made by mutating the V_{L} domain of a V_{H}/V_{L} unit of an antibody that binds a first epitope and selecting the mutant V_{H}/V_{L} unit that can bind the first epitope and a second epitope. For example, in one embodiment, one or more solvent accessible amino acid residue(s) of the light chain CDRs are be randomly or selectively substituted with one or more other amino acid residues(s) prior to screening the mutated V_{H}/V_{L} unit for binding to a second epitope.

An “inflammasome-mediated disease” refers to any disease where IL-1β and/or IL-18 are elevated relative to normal, uninflammed tissue. Generally, in an inflammasome-mediated disease, caspase-1 processing and/or activation is involved/elevated relative to uninduced control cells. Caspase-1 activity can be measured using commercially available assay kits, e.g., Caspase 1 Fluorometric Assay Kit ((Cat. No. ab394120; AbCam, Cambridge, Mass.), Caspase-1 Colorimetric Assay (Cat. No. BF14100; R&D Systems), etc.

In general, a disease or condition can be considered an IL-1β related disease or condition if it is associated with elevated levels of IL-1β in bodily fluids or tissue or if cells or tissues taken from the body produce elevated levels of IL-1β in culture. Similarly, a disease or condition can be considered an IL-18 related disease or condition if it is associated with elevated levels of IL-18 in bodily fluids or tissue or if cells or tissues taken from the body produce elevated levels of IL-18 in culture. Thus, an IL-1β/IL-18 related disease or condition is associated with elevated levels of IL-1β and IL-18 in bodily fluids or tissue or if cells or tissues taken from the body produce elevated levels of both cytokines in culture.

Immune and inflammatory diseases include: chronic obstructive pulmonary disease (COPD), rheumatoid arthritis, osteoarthritis, juvenile chronic arthritis, spondyloarthropathies, systemic sclerosis (scleroderma), idiopathic inflammatory myopathies (dermatomyositis), systemic vasculitis, sarcoidosis, autoimmune hemolytic anemia (immune pancytopenia, paroxysmal nocturnal hemoglobinuria), autoimmune thrombocytopenia (idiopathic thrombocytopenic purpura, immune-mediated thrombocytopenia), thyroiditis (Grave's disease, Hashimoto's thyroiditis, juvenile lymphocytic thyroiditis, atrophic thyroiditis) autoimmune inflammatory diseases (e.g., allergic encephalomyelitis, multiple sclerosis, insulin-dependent diabetes mellitus, autoimmune uveoretinitis, thyrotoxicosis, autoimmune thyroid disease, pernicious-anemia, autograft rejection, diabetes mellitus, and immune-mediated renal disease (glomerulonephritis, tubulointerstitial nephritis)), demyelinating diseases of the central and peripheral nervous systems such as multiple sclerosis, idiopathic demyelinating polyneuropathy or Guillain-Barré syndrome, and chronic inflammatory demyelinating polyneuropathy; hepatobiliary diseases such as infectious hepatitis (hepatitis A, B, C, D, E and other non-hepatotropic viruses), autoimmune chronic active hepatitis, primary biliary cirrhosis, granulomatous hepatitis, and sclerosing cholangitis, gluten-sensitive enteropathy, and Whipple's disease; autoimmune or immune-mediated skin diseases including bullous skin diseases, erythema multiforme and contact dermatitis, psoriasis; allergic diseases such as asthma, allergic rhinitis, atopic dermatitis, vernal conjunctivitis, eczema, food hypersensitivity and urticaria; immunologic diseases of the lung such as eosinophilic pneumonia, idiopathic pulmonary fibrosis and hypersensitivity pneumonitis; transplantation associated disease including graft rejection and graft-versus-host-disease.

Immune related and inflammatory diseases are the manifestations or consequences of fairly complex, often multiple interconnected biological pathways which in normal physiology are critical to respond to insult or injury, initiate repair from insult or injury, and mount innate and acquired defense against foreign organisms. Disease or pathology occurs when these normal physiological pathways cause additional insult or injury either as directly related to the intensity of the response, as a consequence of abnormal regulation or excessive stimulation, as a reaction to self, or a combination of these.

Examples of IL-1β related diseases are acute pancreatitis; ALS; cachexia/anorexia, including AIDS-induced cachexia; asthma and other pulmonary diseases; autoimmune vasculitis; CIAS1 Associated Periodic Syndromes (CAPS); Neonatal Onset Multisystem Inflammatory Disorder (NOMID/CINCA), systemic onset juvenile idiopathic arthritis, Stills disease, Muckle-Wells syndrome, chronic fatigue syndrome; Clostridium associated illnesses, including Clostridium-associated diarrhea; coronary conditions and indications, including congestive heart failure, coronary restenosis, myocardial infarction, myocardial dysfunction (e.g., related to sepsis), and coronary artery bypass graft; cancers, such as multiple myeloma and myelogenous (e.g., AML and CML) and other leukemias, as well as tumor metastasis; diabetes (e.g., insulin diabetes); endometriosis; familial Cold Autoinflammatory Syndrome (FCAS); familial mediterranean fever (FMF); fever; fibromyalgia; glomerulonephritis; graft versus host disease/transplant rejection; hemohorragic shock; hyperalgesia; inflammatory bowel disease; inflammatory conditions of a joint, including psoriatic arthritis (as well as osteoarthritis and rheumatoid arthritis); inflammatory eye disease, as may be associated with, for example, corneal transplant; ischemia, including cerebral ischemia (e.g., brain injury as a result of trauma, epilepsy, hemorrhage or stroke, each of which may lead to neurodegeneration); Kawasaki's disease; learning impairment; lung diseases (e.g., ARDS); myopathies (e.g., muscle protein metabolism, especially in sepsis); neurotoxicity (e.g., as induced by HIV); osteoporosis; pain, including cancer-related pain; Parkinson's disease; periodontal disease; pre-term labor; psoriasis; reperfusion injury; side effects from radiation therapy; sleep disturbance; temporal mandibular joint disease; tumor necrosis factor receptor-associated periodic fever syndrome (TRAPS); uveitis; or an inflammatory condition resulting from strain, sprain, cartilage damage, trauma, orthopedic surgery, infection or other disease processes.

Interleukin 18 plays a critical role in the pathology associated with a variety of diseases involving immune and inflammatory elements. These diseases include, but are not limited to, rheumatoid arthritis, osteoarthritis, juvenile chronic arthritis, Lyme arthritis, psoriatic arthritis, reactive arthritis, spondyloarthropathy, lupus (e.g., Systemic Lupus Erythematosus, and Lupus Nephritis), Crohn's disease, ulcerative colitis, inflammatory bowel disease, insulin dependent diabetes mellitus, thyroiditis, asthma, allergic diseases, psoriasis, psoriasis type 1, psoriasis type 2, dermatitis scleroderma, graft versus host disease, organ transplant rejection, acute or chronic immune disease associated with organ transplantation, sarcoidosis, atherosclerosis, disseminated intravascular coagulation, Kawasaki's disease, Grave's disease, nephrotic syndrome, chronic fatigue syndrome, Wegener's granulomatosis, Henoch-Schoenlein purpurea, microscopic vasculitis of the kidneys, chronic active hepatitis, uveitis, septic shock, toxic shock syndrome, sepsis syndrome, cachexia, infectious diseases, parasitic diseases, acute transverse myelitis, Huntington's chorea, Parkinson's disease, Alzheimer's disease, stroke, primary biliary cirrhosis, hemolytic anemia, malignancies, heart failure, myocardial infarction, Addison's disease, sporadic, polyglandular deficiency type I and polyglandular deficiency type II, Schmidt's syndrome, adult respiratory distress syndrome, alopecia, alopecia greata, seronegative arthopathy, arthropathy, Reiter's disease, psoriatic arthropathy, ulcerative colitic arthropathy, enteropathic synovitis, chlamydia, yersinia and salmonella associated arthropathy, spondyloarthopathy, atheromatous diseasel arteriosclerosis, atopic allergy, autoimmune bullous disease, pemphigus vulgaris, pemphigus foliaceus, pemphigoid, linear IgA disease, autoimmune haemolytic anemia, Coombs positive haemolytic anemia, acquired pernicious anemia, juvenile pernicious anemia, myalgic encephalitis/Royal Free Disease. chronic mucocutaneous candidiasis, giant cell arteritis, primary sclerosing hepatitis, cryptogenic autoimmune hepatitis, Acquired Immunodeficiency Disease Syndrome, Acquired Immunodeficiency Related Diseases, Hepatitis C, common varied immunodeficiency, common variable hypogammaglobulinemia, dilated cardiomyopathy, female infertility, ovarian failure, premature ovarian failure, fibrotic lung disease, cryptogenic fibrosing alveolitis, post-inflammatory interstitial lung disease, interstitial pneumonitis, connective tissue disease associated interstitial lung disease, mixed connective tissue disease associated lung disease, systemic sclerosis associated interstitial lung disease, rheumatoid arthritis associated interstitial lung disease, systemic lupus erythematosus associated lung disease, dermatomyositis/polymyositis associated lung disease, Sjögren's disease associated lung disease, ankylosing spondylitis associated lung disease, vasculitic diffuse lung disease, haemosiderosis associated lung disease, drug-induced interstitial lung disease, radiation fibrosis, bronchiolitis obliterans, chronic eosinophilic pneumonia, lymphocytic infiltrative lung disease, postinfectious interstitial lung disease, gouty arthritis, autoimmune hepatitis, type-1 autoimmune hepatitis, classical autoimmune or lupoid hepatitis, type-2 autoimmune hepatitis, anti-LKM antibody hepatitis, autoimmune mediated hypoglycemia, type B insulin resistance with acanthosis nigricans, hypoparathyroidism, acute immune disease associated with organ transplantation, chronic immune disease associated with organ transplantation, osteoarthrosis, primary sclerosing cholangitis, idiopathic leucopaenia, autoimmune neutropenia, renal disease NOS, glomerulonephritides, microscopic vasulitis of the kidneys, Lyme disease, discoid lupus erythematosus, male infertility idiopathic or NOS, sperm autoimmunity, all subtypes of multiple sclerosis, sympathetic ophthalmia, pulmonary hypertension secondary to connective tissue disease, Goodpasture's syndrome, pulmonary manifestation of polyarteritis nodosa, acute rheumatic fever, rheumatoid spondylitis, Still's disease, systemic sclerosis, Sjogren's syndrome, Takayasu's disease/arteritis, autoimmune thrombocytopenia, idiopathic thrombocytopenia, autoimmune thyroid disease, hyperthyroidism, goitrous autoimmune hypothyroidism or Hashimoto's disease, atrophic autoimmune hypothyroidism, primary myxoedema, phacogenic uveitis, primary vasculitis, vitiligo, acute liver disease, chronic liver diseases, allergy and asthma, mental disorders, depression, schizophrenia, Th2 Type and Th1 Type mediated diseases, Chronic Obstructive Pulmonary Disease (COPD), inflammatory, autoimmune and bone diseases.

The present antibodies and fragments can also be used to treat or prevent IL-1β related, or IL-18 related, or autoinflammatory, or autoimmune or inflammation or immune diseases.

Though the genesis of these diseases often involved multistep pathways and often multiple different biological systems/pathways, intervention at critical points in one or more of these pathways can have an ameliorative or therapeutic effect. Therapeutic intervention can occur by either antagonism of a detrimental process/pathway or stimulation of a beneficial process/pathway.

T lymphocytes (T cells) are an important component of a mammalian immune response. T cells recognize antigens which are associated with a self-molecule encoded by genes within the major histocompatibility complex (MHC). The antigen may be displayed together with MHC molecules on the surface of antigen presenting cells, virus infected cells, cancer cells, grafts, etc. The T cell system eliminates these altered cells which pose a health threat to the host animal. T cells include helper T cells and cytotoxic T cells. Helper T cells proliferate extensively following recognition of an antigen-MHC complex on an antigen presenting cell. Helper T cells also secrete a variety of cytokines, e.g., lymphokines, which play a central role in the activation of B cells, cytotoxic T cells and a variety of other cells which participate in the immune response.

In many immune responses, inflammatory cells infiltrate the site of injury or infection. The migrating cells may be neutrophilic, eosinophilic, monocytic or lymphocytic as can be determined by histologic examination of the affected tissues. See, e.g., Current Protocols in Immunology, ed. John E. Coligan, 1994, John Wiley & Sons, Inc. Many immune related diseases are known and have been extensively studied. Such diseases include immune-mediated inflammatory diseases (e.g., rheumatoid arthritis, immune mediated renal disease, hepatobiliary diseases, inflammatory bowel disease (IBD), psoriasis, and asthma), non-immune-mediated inflammatory diseases, infectious diseases, immunodeficiency diseases, neoplasia, and graft rejection, etc. In the area of immunology, targets were identified for the treatment of inflammation and inflammatory disorders. In the area of immunology, targets have been identified herein for the treatment of inflammation and inflammatory disorders. Immune related diseases, in one instance, could be treated by suppressing the immune response. Using neutralizing antibodies that inhibit molecules having immune stimulatory activity would be beneficial in the treatment of immune-mediated and inflammatory diseases. Molecules which inhibit the immune response can be utilized (proteins directly or via the use of antibody agonists) to inhibit the immune response and thus ameliorate immune related disease.

As used herein, the term “immunoadhesin” designates molecules which combine the binding specificity of a heterologous protein (an “adhesin”) with the effector functions of immunoglobulin constant domains. Structurally, the immunoadhesins comprise a fusion of an amino acid sequence with a desired binding specificity, which amino acid sequence is other than the antigen recognition and binding site of an antibody (i.e., is “heterologous”), and an Fc region (e.g., CH2 and/or CH3 sequence of an IgG). Exemplary adhesin sequences include contiguous amino acid sequences that comprise a portion of a receptor (e.g., extracellular domain) or a ligand that binds to a protein of interest. Adhesin sequences can also be sequences that bind a protein of interest, but are not receptor or ligand sequences (e.g., adhesin sequences in peptibodies). Such polypeptide sequences can be selected or identified by various methods, include phage display techniques and high throughput sorting methods. The immunoglobulin constant domain sequence in the immunoadhesin can be obtained from any immunoglobulin, such as IgG-1, IgG-2, IgG-3, or IgG-4 subtypes, IgA (including IgA-1 and IgA-2), IgE, IgD, or IgM. Exemplary molecules are the bispecific CD4-IgG molecules described in Berg et al., 1991, PNAS (USA) 88:4723- and Chamow et al., 1994, J. Immunol. 153:4268.

An “isolated” antibody is one that has been identified and separated and/or recovered from a component of its natural environment. Contaminant components of its natural environment are materials that would interfere with diagnostic or therapeutic uses for the antibody, and may include enzymes, hormones, and other proteinaceous or nonproteinaceous solutes. In some embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

An “isolated” nucleic acid molecule is a nucleic acid molecule that is identified and separated from at least one contaminant nucleic acid molecule with which it is ordinarily associated in the natural source of the antibody nucleic acid. An isolated nucleic acid molecule is other than in the form or setting in which it is found in nature. Isolated nucleic acid molecules therefore are distinguished from the nucleic acid molecule as it exists in natural cells. However, an isolated nucleic acid molecule includes a nucleic acid molecule contained in cells that ordinarily express the antibody where, for example, the nucleic acid molecule is in a chromosomal location different from that of natural cells.

The term “knob-into-hole” or “KnH” as mentioned herein refers to the technology directing the selectively pairing of two polypeptides together in vitro or in vivo by introducing a pertuberance (knob) into one polypeptide and a cavity (hole) into the other polypeptide at an interface in which they interact. For example, KnHs have been introduced in the Fc:Fc binding interfaces, C_{L}:C_{H}1 interfaces or V_{H}/V_{L} interfaces of antibodies (e.g., US20007/0178552, WO 96/027011, WO 98/050431 and Zhu et al. (1997) Protein Science 6:781-788). This is especially useful in driving the pairing of two different heavy chains together during the manufacture of multispecific antibodies. For example, multispecific antibodies having KnH in their Fc regions can further comprise single variable domains linked to each Fc region, or further comprise different heavy chain variable domains that pair with similar or different light chain variable domains. In fact, KnH technology can be used to pair two different receptor extracellular domains together or any other polypeptide sequences that comprises different target recognition sequences (e.g., including affibodies, peptibodies and other Fc fusions).

The expression “linear antibodies” generally refers to the antibodies described in Zapata et al., Protein Eng. 8(10):1057-1062 (1995). Briefly, these antibodies comprise a pair of tandem Fd segments (V_{H}-C_{H}1-V_{H}-C_{H}1) which, together with complementary light chain polypeptides, form a pair of antigen binding regions. Linear antibodies can be bispecific or monospecific.

The term “mammal” includes any animals classified as mammals, including humans, cows, horses, dogs, and cats. In one embodiment the mammal is a human.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations that typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al., 1975, Nature 256:495, or may be made by recombinant DNA methods (see, for example, U.S. Pat. No. 4,816,567). The “monoclonal antibodies” may also be isolated from phage antibody libraries using the techniques described in Clackson et al., 1991, Nature 352:624-628 and Marks et al., 1991, J. Mol. Biol. 222:581-597, for example.

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., 1984, Proc. Natl. Acad. Sci. USA 81:6851-6855).

The term “multispecific antibody” is used in the broadest sense and refers to an antibody that has polyeptopic specificity. Such multispecific antibodies include, but are not limited to, an antibody comprising a heavy chain variable domain (V_{H}) and a light chain variable domain (V_{L}), where the V_{H}V_{L} unit has polyepitopic specificity, antibodies having two or more V_{L} and V_{H} domains with each V_{H}V_{L} unit binding to a different epitope, antibodies having two or more single variable domains with at least two single variable domains binding to different epitopes, full length antibodies, antibody fragments such as Fab, Fv, dsFv, scFv, diabodies, tandem antibodies, linear antibodies and triabodies, antibody fragments that have been linked covalently or bind to each other through non-covalent interactions. Other examples of antibody formats have been used or may be used to create multispecific antibodies include, but are not limited to, Fc fusions of diabodies, tandem antibodies, and single chain antibodies (e.g., Db-Fc, taDb-Fc, taDb-CH3 and (scFV)4-Fc), knob-N-hole (KnH) antibodies, octopus antibodies and DAF antibodies.

“Multispecific Molecule” as used herein refers to a molecule that has polyepitopic specificity. “Polyepitopic specificity” refers to the ability to specifically bind to \at least two different epitopes on one target molecule or on a different target molecules. “Monospecific” refers to the ability to bind only one epitope. According to one embodiment a multispecific molecule binds to each epitope with an affinity of 5 μM to 0.001 pM, 3 μM to 0.001 pM, 1 μM to 0.001 pM, 0.5 μM to 0.001 pM or 0.1 μM to 0.001 pM. The term “bispecific” as used herein refers to the ability to bind two epitopes (e.g., an anti-IL-1β/IL-18 bispecific antibody). Examples of molecules that support or can be engineered to support polyepitopic specificity include, but is not limited to, antibodies, affibodies, immunoadhesins, peptibodies and other Fc fusions.

The term “octopus” antibody or antibodies as used herein refers to multivalent antibodies comprising an Fc region and two or more antigen binding sites amino-terminal to the Fc region (e.g., WO01/77342, Wu et al. (2007) Nature Biotechnology, and WO 2007/024715). In one preferred embodiment, the configuration of a polypeptide of the antibody is VD1-(X1)n-VD2-(X2)n-Fc, wherein VD1 is a first variable domain, VD2 is a second variable domain, Fc is one polypeptide chain of an Fc region, X1 and X2 represent an amino acid or polypeptide, and n is 0 or 1. In one embodiment, X1 or X2 is a CH1 domain, a portion of a CH1 domain, some other linker sequence such as a GS linker or some combination thereof (e.g., page 5 of WO 2007/024715).

A nucleic acid is “operably linked,” as used herein, when it is placed into a functional relationship with another nucleic acid sequence. For example, DNA for a presequence or secretory leader is operably linked to DNA for a antibody if it is expressed as a preprotein that participates in the secretion of the antibody; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, an enhancer may not have to be contiguous. Linking is accomplished by ligation at convenient restriction sites. If such sites do not exist, synthetic oligonucleotide adaptors or linkers are used in accordance with conventional practice.

“Peptibody” or “peptibodies” refers to a fusion of peptide sequences with an Fc domain. See U.S. Pat. No. 6,660,843, issued Dec. 9, 2003 to Feige et al. (incorporated by reference in its entirety). They include one or more peptides linked to the N-terminus, C-terminus, amino acid sidechains, or to more than one of these sites. Peptibody technology enables design of therapeutic agents that incorporate peptides that target one or more ligands or receptors, tumor-homing peptides, membrane-transporting peptides, and the like. Peptibody technology has proven useful in design of a number of such molecules, including linear and disulfide-constrained peptides, “tandem peptide multimers” (i.e., more than one peptide on a single chain of an Fc domain). See, for example, U.S. Pat. No. 6,660,843; U.S. Pat. App. No. 2003/0195156, published Oct. 16, 2003 (corresponding to WO 02/092620, published Nov. 21, 2002); U.S. Pat. App. No. 2003/0176352, published Sep. 18, 2003 (corresponding to WO 03/031589, published Apr. 17, 2003); U.S. Ser. No. 09/422,838, filed Oct. 22, 1999 (corresponding to WO 00/24770, published May 4, 2000); U.S. Pat. App. No. 2003/0229023, published Dec. 11, 2003; WO 03/057134, published Jul. 17, 2003; U.S. Pat. App. No. 2003/0236193, published Dec. 25, 2003 (corresponding to PCT/US04/010989, filed Apr. 8, 2004); U.S. Ser. No. 10/666,480, filed Sep. 18, 2003 (corresponding to WO 04/026329, published Apr. 1, 2004), each of which is hereby incorporated by reference in its entirety.

For the purposes herein, a “pharmaceutical composition” is one that is adapted and suitable for administration to a mammal, especially a human. Thus, the composition can be used to treat a disease or disorder in the mammal. Moreover, the protein in the composition has been subjected to one or more purification or isolation steps, such that contaminant(s) that might interfere with its therapeutic use have been separated therefrom. Generally, the pharmaceutical composition comprises the therapeutic protein and a pharmaceutically acceptable carrier or diluent. The composition is usually sterile and may be lyophilized. Pharmaceutical preparations are described in more detail below.

“Polynucleotide,” or “nucleic acid,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase or by a synthetic reaction. A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may be further modified after synthesis, such as by conjugation with a label. Other types of modifications include, for example, “caps”, substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, ply-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid or semi-solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl, 2′-fluoro- or 2′-azido-ribose, carbocyclic sugar analogs, a-anomeric sugars, epimeric sugars such as arabinose, xyloses or lyxoses, pyranose sugars, furanose sugars, sedoheptuloses, acyclic analogs, and abasic nucleoside analogs such as methyl riboside. One or more phosphodiester linkage may be replaced by alternative linking groups. These alternative linking groups include, but are not limited to, embodiments wherein phosphate is replaced by P(O)S(“thioate”), P(S)S (“dithioate”), “(O)NR2 (“amidate”), P(O)R, P(O)OR′, CO or CH2 (“formacetal”), in which each R or R′ is independently H or substituted or unsubstituted alkyl (1-20 C.) optionally containing an ether (—O—) linkage, aryl, alkenyl, cycloalkyl, cycloalkenyl, or araldyl. Not all linkages in a polynucleotide need be identical. The preceding description applies to all polynucleotides referred to herein, including RNA and DNA.

“Oligonucleotide,” as used herein, generally refers to short, generally single stranded, generally synthetic polynucleotides that are generally, but not necessarily, less than about 200 nucleotides in length. The terms “oligonucleotide” and “polynucleotide” are not mutually exclusive. The description above for polynucleotides is equally and fully applicable to oligonucleotides.

The term “receptor binding domain” is used to designate any native ligand for a receptor, including cell adhesion molecules, or any region or derivative of such native ligand retaining at least a qualitative receptor binding ability of a corresponding native ligand. This definition, among others, specifically includes binding sequences from ligands for the above-mentioned receptors.

“Secretion signal sequence” or “signal sequence” refers to a nucleic acid sequence encoding a short signal peptide that can be used to direct a newly synthesized protein of interest through a cellular membrane, usually the inner membrane or both inner and outer membranes of prokaryotes. As such, the protein of interest such as the immunoglobulin light or heavy chain polypeptide is secreted into the periplasm of the prokaryotic host cells or into the culture medium. The signal peptide encoded by the secretion signal sequence may be endogenous to the host cells, or they may be exogenous, including signal peptides native to the polypeptide to be expressed. Secretion signal sequences are typically present at the amino terminus of a polypeptide to be expressed, and are typically removed enzymatically between biosynthesis and secretion of the polypeptide from the cytoplasm. Thus, the signal peptide is usually not present in a mature protein product.

The expression “single domain antibodies” (sdAbs) or “single variable domain (SVD) antibodies” generally refers to antibodies in which a single variable domain (V_{H} or V_{L}) can confer antigen binding. In other words, the single variable domain need not interact with another variable domain in order to bind the target antigen. Examples of single domain antibodies include, but is not limited to, those derived from nature such as camelids (lamas and camels) and cartilaginous fish (e.g., nurse sharks) and those derived from recombinant methods from humans and mouse antibodies (Nature (1989) 341:544-546; Dev Comp Immunol (2006) 30:43-56; Trend Biochem Sci (2001) 26:230-235; Trends Biotechnol (2003):21:484-490; WO 2005/035572; WO 03/035694; Febs Lett (1994) 339:285-290; WO00/29004; WO 02/051870).

As used herein, a “therapeutic antibody” is an antibody that is effective in treating a disease or disorder in a mammal with or predisposed to the disease or disorder. Exemplary therapeutic antibodies include the anti-IL-1β and anti-IL-18 antibodies of the present invention, including the anti-IL-1β and anti-IL-18 bispecific antibodies of the present invention, as well as antibodies including rhuMAb 4D5 (HERCEPTIN®) (Carter et al., 1992, Proc. Natl. Acad. Sci. USA, 89:4285-4289, U.S. Pat. No. 5,725,856); anti-CD20 antibodies such as chimeric anti-CD20 “C2B8” as in U.S. Pat. No. 5,736,137 (RITUXAN®), a chimeric or humanized variant of the 2H7 antibody as in U.S. Pat. No. 5,721,108, B1 or Tositumomab (BEXXAR®); anti-IL-8 (St John et al., 1993, Chest, 103:932, and International Publication No. WO 95/23865); anti-VEGF antibodies including humanized and/or affinity matured anti-VEGF antibodies such as the humanized anti-VEGF antibody huA4.6.1 AVASTIN™ (Kim et al., 1992, Growth Factors, 7:53-64, International Publication No. WO 96/30046, and WO 98/45331, published Oct. 15, 1998); anti-PSCA antibodies (WO01/40309); anti-CD40 antibodies, including S2C6 and humanized variants thereof (WO00/75348); anti-CD 11a (U.S. Pat. No. 5,622,700, WO 98/23761, Steppe et al, 1991, Transplant Intl. 4:3-7, and Hourmant et al., 1994, Transplantation 58:377-380); anti-IgE (Presta et al., 1993, J. Immunol. 151:2623-2632, and International Publication No. WO 95/19181); anti-CD18 (U.S. Pat. No. 5,622,700, issued Apr. 22, 1997, or as in WO 97/26912, published Jul. 31, 1997); anti-IgE (U.S. Pat. No. 5,714,338, issued Feb. 3, 1998 or U.S. Pat. No. 5,091,313, issued Feb. 25, 1992, WO 93/04173 published Mar. 4, 1993, or International Application No. PCT/US98/13410 filed Jun. 30, 1998, U.S. Pat. No. 5,714,338); anti-Apo-2 receptor antibody (WO 98/51793 published Nov. 19, 1998); anti-TNF-α antibodies including cA2 (REMICADE®), CDP571 and MAK-195 (See, U.S. Pat. No. 5,672,347 issued Sep. 30, 1997, Lorenz et al. 1996, J. Immunol. 156(4):1646-1653, and Dhainaut et al. 1995, Crit. Care Med. 23(9):1461-1469); anti-Tissue Factor (TF) (European Patent No. 0 420 937 B1 granted Nov. 9, 1994); anti-human γ₄-β₇ integrin (WO 98/06248 published Feb. 19, 1998); anti-EGFR (chimerized or humanized 225 antibody as in WO 96/40210 published Dec. 19, 1996); anti-CD3 antibodies such as OKT3 (U.S. Pat. No. 4,515,893 issued May 7, 1985); anti-CD25 or anti-tac antibodies such as CHI-621 (SIMULECT®) and (ZENAPAX®) (See U.S. Pat. No. 5,693,762 issued Dec. 2, 1997); anti-CD4 antibodies such as the cM-7412 antibody (Choy et al. 1996, Arthritis Rheum 39(1):52-56); anti-CD52 antibodies such as CAMPATH-1H (Riechmann et al. 1988, Nature 332:323-337; anti-Fc receptor antibodies such as the M22 antibody directed against FcγRI as in Graziano et al. 1995, J. Immunol. 155(10):4996-5002; anti-carcinoembryonic antigen (CEA) antibodies such as hMN-14 (Sharkey et al. 1995, Cancer Res. 55(23 Suppl): 5935s-5945s; antibodies directed against breast epithelial cells including huBrE-3, hu-Mc 3 and CHL6 (Ceriani et al. 1995, Cancer Res. 55(23): 5852s-5856s; and Richman et al. 1995, Cancer Res. 55(23 Supp): 5916s-5920s); antibodies that bind to colon carcinoma cells such as C242 (Litton et al. 1996, Eur J. Immunol. 26(1): 1-9); anti-CD38 antibodies, e.g. AT 13/5 (Ellis et al. 1995, J. Immunol. 155(2):925-937); anti-CD33 antibodies such as Hu M195 (Jurcic et al. 1995, Cancer Res 55(23 Suppl):5908s-5910s and CMA-676 or CDP771; anti-CD22 antibodies such as LL2 or LymphoCide (Juweid et al. 1995, Cancer Res 55(23 Suppl):5899s-5907s; anti-EpCAM antibodies such as 17-1A (PANOREX®); anti-GpIIb/IIIa antibodies such as abciximab or c7E3 Fab (REOPRO®); anti-RSV antibodies such as MEDI-493 (SYNAGIS®); anti-CMV antibodies such as PROTOVIR®; anti-HIV antibodies such as PRO542; anti-hepatitis antibodies such as the anti-Hep B antibody OSTAVIR®; anti-CA 125 antibody OvaRex; anti-idiotypic GD3 epitope antibody BEC2; anti-αvβ3 antibody VITAXIN®; anti-human renal cell carcinoma antibody such as ch-G250; ING-1; anti-human 17-IA antibody (3622W94); anti-human colorectal tumor antibody (A33); anti-human melanoma antibody R24 directed against GD3 ganglioside; anti-human squamous-cell carcinoma (SF-25); and anti-human leukocyte antigen (HLA) antibodies such as Smart ID10 and the anti-HLA DR antibody Oncolym (Lym-1).

“Target molecule” refers to a molecule that is capable of binding a target recognition site. Examples of target molecule:target recognition site interactions include antigen:antibody variable domain interactions, receptor:ligand interactions, ligand:receptor interactions, adhesin:adhesin interactions, biotin:strepavidin interactions, etc. In one embodiment, the target molecule is a biological molecule.

The term “therapeutically effective amount” refers to an amount of a composition of this invention effective to “alleviate” or “treat” a disease or disorder in a subject or mammal. In one embodiment, “therapeutically effective amount” is intended to include an amount of the antibodies described herein alone or in combination with other active ingredients effective to inhibit or decrease IL-1beta and IL-18 binding to their receptors or effective to treat or prevent inflammatory disorders in a subject in need thereof.

“Treatment” (and grammatical variations thereof such as “treat” or “treating”) refers to clinical intervention in an attempt to alter the natural course of the subject being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, antibodies of the invention are used to delay development of a disease or to slow the progression of a disease. Generally, treatment of a disease or disorder involves the lessening of one or more symptoms or medical problems associated with the disease or disorder. In some embodiments, antibodies and compositions of this invention can be used to prevent the onset or reoccurrence of the disease or disorder in a subject or mammal. For example, in a subject with autoimmune disease, an antibody of this invention can be used to prevent or treat flare-ups. Consecutive treatment or administration refers to treatment on at least a daily basis without interruption in treatment by one or more days. Intermittent treatment or administration, or, treatment or administration in an intermittent fashion, refers to treatment that is not consecutive, but rather cyclic in nature. The treatment regime herein may be either consecutive or intermittent.

The term “variable” refers to the fact that certain segments of the variable domains differ extensively in sequence among antibodies. The V domain mediates antigen binding and defines specificity of a particular antibody for its particular antigen. However, the variability is not evenly distributed across the amino acid span of a variable domain. Instead, the V region consist of relatively invariant stretches called framework regions (FRs) of separated by shorter regions of extreme variability called “hypervariable regions”. The hypervariable regions in one variable domain may cooperate with the hypervariable regions from another chain to contribute to the formation of a antigen-binding site on antibodies, depending on the type of antibody (see Kabat et al., Sequences of Proteins of Immunological Interest, 5th Ed. Public Health Service, National Institutes of Health, Bethesda, Md. (1991)). Constant domains are not typically involved directly in binding an antibody to an antigen, but exhibit various effector functions, such as participation of the antibody in antibody dependent cellular cytotoxicity (ADCC).

A “variant” or “altered” heavy chain, as used herein, generally refers to a heavy chain with reduced disulfide linkage capability, for e.g., wherein at least one cysteine residue has been rendered incapable of disulfide linkage formation. Preferably, said at least one cysteine is in the hinge region of the heavy chain.

The term “vector,” as used herein, is intended to refer to a nucleic acid molecule capable of transporting another nucleic acid to which it has been linked. One type of vector is a “plasmid”, a circular double stranded DNA loop into which additional DNA segments may be ligated. Another type of vector is a phage vector. Another type of vector is a viral vector, wherein additional DNA segments may be ligated into the viral genome. Certain vectors are capable of autonomous replication in a host cell into which they are introduced (for example, bacterial vectors having a bacterial origin of replication and episomal mammalian vectors). Other vectors (for example, non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “recombinant vectors”). In general, expression vectors of utility in recombinant DNA techniques are often in the form of plasmids. In the present specification, “plasmid” and “vector” may be used interchangeably as the plasmid is the most commonly used form of vector.

An antibody that “selectively binds” a target molecule with significantly better affinity than it binds to other molecules that are not the target molecule. The relative binding and/or binding affinity may be demonstrated in a variety of methods accepted in the art including, but not limited to: enzyme linked immunosorbent assay (ELISA) and fluorescence activated cell sorting (FACS). In some embodiments, the antibody of the invention binds a target molecule with at least about 1 log higher concentration reactivity than it binds to a non-target molecule, as determined by an ELISA.

### I. Exemplary Antibodies

Soluble human IL-1β or human IL-18, or fragments thereof, optionally conjugated to other molecules, can be used as immunogens for generating antibodies. Alternatively, or additionally, cells expressing human IL-1β or human IL-18 can be used as the immunogen. Such cells can be derived from a natural source or may be cells that have been transformed by recombinant techniques to express human IL-1β or human IL-18. Other forms of human IL-1β or human IL-18 useful for preparing antibodies will be apparent to those in the art.

A. Polyclonal Antibodies

Polyclonal antibodies are preferably raised in animals by multiple subcutaneous (sc) or intraperitoneal (ip) injections of the relevant antigen and an adjuvant. It may be useful to conjugate the relevant antigen to a protein that is immunogenic in the species to be immunized, e.g., keyhole limpet hemocyanin, serum albumin, bovine thyroglobulin, or soybean trypsin inhibitor using a bifunctional or derivatizing agent, for example, maleimidobenzoyl sulfosuccinimide ester (conjugation through cysteine residues), N-hydroxysuccinimide (through lysine residues), glutaraldehyde, succinic anhydride, SOCl₂, or R¹N═C═NR, where R and R′ are different alkyl groups.

Animals are immunized against the antigen, immunogenic conjugates, or derivatives by combining, for example, 100 μg or 5 μg of the protein or conjugate (for rabbits or mice, respectively) with 3 volumes of Freund's complete adjuvant and injecting the solution intradermally at multiple sites. Approximately one month later, the animals are boosted with ⅕ to 1/10 the original amount of peptide or conjugate in Freund's complete adjuvant by subcutaneous injection at multiple sites. Seven to 14 days later the animals are bled and the serum is assayed for antibody titer. Animals are boosted until the titer plateaus. Preferably, the animal is boosted with the conjugate of the same antigen, but conjugated to a different protein and/or through a different cross-linking reagent. Conjugates also can be made in recombinant cell culture as protein fusions. Also, aggregating agents such as alum are suitably used to enhance the immune response.

B. Monoclonal Antibodies

Monoclonal antibodies may be made using the hybridoma method first described by Kohler et al., 1975, Nature, 256:495, or may be made by recombinant DNA methods (See, for example, U.S. Pat. No. 4,816,567).

In the hybridoma method, a mouse or other appropriate host animal, such as a hamster or macaque monkey, is immunized as hereinabove described to elicit lymphocytes that produce or are capable of producing antibodies that will specifically bind to the protein used for immunization. Alternatively, lymphocytes may be immunized in vitro. Lymphocytes then are fused with myeloma cells using a suitable fusing agent, such as polyethylene glycol, to form a hybridoma cell (Goding, 1986, Monoclonal Antibodies: Principles and Practice, pp. 59-103 (Academic Press)).

The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Preferred myeloma cells are those that fuse efficiently, support stable high-level production of antibody by the selected antibody-producing cells, and are sensitive to a medium such as HAT medium. Among these, preferred myeloma cell lines are murine myeloma lines, such as those derived from MOPC-21 and MPC-11 mouse tumors available from the Salk Institute Cell Distribution Center, San Diego, Calif. USA, and SP-2 or X63-Ag8-653 cells available from the American Type Culture Collection, Rockville, Md. USA. Human myeloma and mouse-human heteromyeloma cell lines also have been described for the production of human monoclonal antibodies (Kozbor, 1984, J. Immunol., 133:3001; Brodeur et al., 1987, Monoclonal Antibody Production Techniques and Applications, pp. 51-63 (Marcel Dekker, Inc., New York)).

Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immunoabsorbent assay (ELISA).

After hybridoma cells are identified that produce antibodies of the desired specificity, affinity, and/or activity, the clones may be subcloned by limiting dilution procedures and grown by standard methods (Goding, supra). Suitable culture media for this purpose include, for example, D-MEM or RPMI-1640 medium. In addition, the hybridoma cells may be grown in vivo as ascites tumors in an animal.

The monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, or affinity chromatography.

DNA encoding the monoclonal antibodies is readily isolated and sequenced using conventional procedures (e.g., by using oligonucleotide probes that are capable of binding specifically to genes encoding the heavy and light chains of the monoclonal antibodies). The hybridoma cells serve as a preferred source of such DNA. Once isolated, the DNA may be placed into expression vectors, which are then transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells. Recombinant production of antibodies will be described in more detail below.

In a further embodiment, antibodies or antibody fragments can be isolated from antibody phage libraries generated using the techniques described in McCafferty et al., 1990, Nature, 348:552-554. Clackson et al., 1991, Nature, 352:624-628, and Marks et al., 1991, J. Mol. Biol., 222:581-597 describe the isolation of murine and human antibodies, respectively, using phage libraries. Subsequent publications describe the production of high affinity (nM range) human antibodies by chain shuffling (Marks et al., 1992, Bio/Technology, 10:779-783), as well as combinatorial infection and in vivo recombination as a strategy for constructing very large phage libraries (Waterhouse et al., 1993, Nuc. Acids. Res., 21:2265-2266). Thus, these techniques are viable alternatives to traditional monoclonal antibody hybridoma techniques for isolation of monoclonal antibodies.

The DNA also may be modified, for example, by substituting the coding sequence for human heavy- and light-chain constant domains in place of the homologous murine sequences (U.S. Pat. No. 4,816,567; Morrison, et al., 1984, Proc. Natl. Acad. Sci. USA, 81:6851), or by covalently joining to the immunoglobulin coding sequence all or part of the coding sequence for non-immunoglobulin material (e.g., protein domains).

Typically such non-immunoglobulin material is substituted for the constant domains of an antibody, or is substituted for the variable domains of one antigen-combining site of an antibody to create a chimeric bivalent antibody comprising one antigen-combining site having specificity for an antigen and another antigen-combining site having specificity for a different antigen.

C. Humanized and Human Antibodies

A humanized antibody has one or more amino acid residues from a source that is non-human. The non-human amino acid residues are often referred to as “import” residues, and are typically taken from an “import” variable domain. Humanization can be performed generally following the method of Winter and co-workers (Jones et al., 1986, Nature, 321:522-525; Riechmann et al., 1988, Nature, 332:323-327; Verhoeyen et al., 1988, Science, 239:1534-1536), by substituting rodent CDRs or CDR sequences for the corresponding sequences of a human antibody. Accordingly, such “humanized” antibodies are chimeric antibodies (U.S. Pat. No. 4,816,567) wherein substantially less than an intact human variable domain has been substituted by the corresponding sequence from a non-human species. In practice, humanized antibodies are typically human antibodies in which some CDR residues and possibly some FR residues are substituted by residues from analogous sites in non-human, for example, rodent antibodies.

The choice of human variable domains, both light and heavy, to be used in making the humanized antibodies is very important to reduce antigenicity. According to the so-called “best-fit” method, the sequence of the variable domain of a rodent antibody is screened against the entire library of known human variable-domain sequences. The human sequence which is closest to that of the rodent is then accepted as the human framework (FR) for the humanized antibody (Sims et al., 1987, J. Immunol., 151:2296; Chothia et al., 1987, J. Mol. Biol., 196:901). Another method uses a particular framework derived from the consensus sequence of all human antibodies of a particular subgroup of light or heavy chains. The same framework may be used for several different humanized antibodies (Carter et al., 1992, Proc. Natl. Acad. Sci. USA, 89:4285; Presta et al., 1993, J. Immunol., 151:2623).

It is further important that antibodies be humanized with retention of high affinity for the antigen and other favorable biological properties. To achieve this goal, according to a preferred method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the CDR residues are directly and most substantially involved in influencing antigen binding.

Alternatively, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (JH) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits et al., 1993, Proc. Natl. Acad. Sci. USA, 90:2551; Jakobovits et al., 1993, Nature, 362:255-258; Bruggermann et al., 1993, Year in Immuno., 7:33; and Duchosal et al., 1992, Nature 355:258. Human antibodies can also be derived from phage-display libraries (Hoogenboom et al., 1991, J. Mol. Biol., 227:381; Marks et al., J. Mol. Biol., 1991, 222:581-597; Vaughan et al., 1996, Nature Biotech 14:309).

i. Chimeric and Humanized Antibodies

In certain embodiments, an antibody provided herein is a chimeric antibody. Certain chimeric antibodies are described, e.g., in U.S. Pat. No. 4,816,567; and Morrison et al., Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing SDR (a-CDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

ii. Human Antibodies

In certain embodiments, an antibody provided herein is a human antibody. Human antibodies can be produced using various techniques known in the art. Human antibodies are described generally in van Dijk and van de Winkel, Curr. Opin. Pharmacol. 5: 368-74 (2001) and Lonberg, Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HuMAB® technology; U.S. Pat. No. 7,041,870 describing K-M MOUSE® technology, and U.S. Patent Application Publication No. US 2007/0061900, describing VELOCIMOUSE® technology). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3):185-91 (2005).

Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

D. Multispecific Antibodies

Multispecific antibodies have binding specificities for at least two different antigens. While such molecules normally will only bind two antigens (e.g., bispecific antibodies, BsAbs), antibodies with additional specificities such as trispecific antibodies are encompassed by this expression when used herein. Examples of BsAbs include those with one antigen binding site directed against IL-1β and another antigen binding site directed against IL-18. In some embodiments, the BsAbs comprise a first binding specificity for IL-1β or IL-18 and a second binding specificity for an activating receptor having a cytoplasmic ITAM motif. An ITAM motif structure possesses two tyrosines separate by a 9-11 amino acid spacer. A general consensus sequence is YxxL/I(x)₆₋₈YxxL (Isakov, N., 1997, J. Leukoc. Biol., 61:6-16). Exemplary activating receptors include FcεRI, FcγRIII, FcγRI, FcγRIIA, and FcγRIIC. Other activating receptors include, e.g., CD3, CD2, CD10, CD161, DAP-12, KAR, KARAP, FcεRII, Trem-1, Trem-2, CD28, p44, p46, B cell receptor, LMP2A, STAM, STAM-2, GPVI, and CD40 (See, e.g., Azzoni, et al., 1998, J. Immunol. 161:3493; Kita, et al., 1999, J. Immunol. 162:6901; Merchant, et al., 2000, J. Biol. Chem. 74:9115; Pandey, et al., 2000, J. Biol. Chem. 275:38633; Zheng, et al., 2001, J. Biol. Chem. 276:12999; Propst, et al., 2000, J. Immunol. 165:2214).

In one embodiment, a BsAb comprises a first binding specificity for IL-1β and a second binding specificity for IL-18. Bispecific antibodies can be prepared as full length antibodies or antibody fragments (for example, F(ab′)₂ bispecific antibodies). Bispecific antibodies may additionally be prepared as knobs-in-holes or hingeless antibodies. Bispecific antibodies are reviewed in Segal et al., 2001, J. Immunol. Methods 248:1-6.

Methods for making bispecific antibodies are known in the art. Traditional production of full length bispecific antibodies is based on the coexpression of two immunoglobulin heavy chain-light chain pairs, where the two chains have different specificities (Millstein et al., 1983, Nature, 305:537-539). Because of the random assortment of immunoglobulin heavy and light chains, these hybridomas (quadromas) produce a potential mixture of 10 different antibody molecules, of which only one has the correct bispecific structure. Purification of the correct molecule, usually done by affinity chromatography steps, is rather cumbersome, and the product yields are low. Similar procedures are disclosed in WO 93/08829, and in Traunecker et al., 1991, EMBO J., 10:3655-3659.

According to a different approach, antibody variable domains with the desired binding specificities (antibody-antigen combining sites) are fused to immunoglobulin constant domain sequences. The fusion can be with an immunoglobulin heavy chain constant domain, comprising at least part of the hinge, CH2, and CH3 regions. It is preferred to have the first heavy-chain constant region (CH1) containing the site necessary for light chain binding, present in at least one of the fusions. DNAs encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three antibody fragments in embodiments when unequal ratios of the three antibody chains used in the construction provide the optimum yields. It is, however, possible to insert the coding sequences for two or all three antibody chains in one expression vector when the expression of at least two antibody chains in equal ratios results in high yields or when the ratios are of no particular significance.

In another embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile method of separation. This approach is disclosed in WO 94/04690. For further details of methods for generating bispecific antibodies, see, for example, Suresh et al., 1986, Methods in Enzymology, 121:210.

According to another approach described in WO96/27011, the interface between a pair of antibody molecules can be engineered to maximize the percentage of heterodimers that are recovered from recombinant cell culture. The preferred interface comprises at least a part of the CH3 domain of an antibody constant domain. In this method, one or more small amino acid side chains from the interface of the first antibody molecule are replaced with larger side chains (for example, tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Such antibodies have, for example, been proposed to target immune system cells to unwanted cells (U.S. Pat. No. 4,676,980), and for treatment of HIV infection (WO 91/00360, WO 92/200373, and EP 03089). Heteroconjugate antibodies may be made using any convenient cross-linking methods. Suitable cross-linking agents are well known in the art, and are disclosed, for example, in U.S. Pat. No. 4,676,980, along with a number of cross-linking techniques.

Antibodies with more than two valencies are also contemplated. For example, trispecific antibodies can be prepared According to Tutt et al., 1991, J. Immunol. 147: 60.

Engineered antibodies with three or more functional antigen binding sites, including “Octopus antibodies,” are also included herein (see, e.g. US 2006/0025576A1).

The antibody or fragment herein also includes a “Dual Acting FAb” or “DAF” comprising an antigen binding site that binds to IL-1β as well as IL-18 (see, US 2008/0069820, for example).

E. Antibodies with Variant Hinge Regions

The antibodies of the present invention may also comprise variant heavy chains, for example as described in application Ser. No. 10/697,995, filed Oct. 30, 2003. Antibodies comprising variant heavy chains comprise an alteration of at least one disulfide-forming cysteine residue, such that the cysteine residue is incapable of forming a disulfide linkage. In one aspect, said cysteine(s) is of the hinge region of the heavy chain (thus, such a hinge region is referred to herein as a “variant hinge region” and may additionally be referred to as “hingeless”).

In some aspects, such immunoglobulins lack the complete repertoire of heavy chain cysteine residues that are normally capable of forming disulfide linkages, either intermolecularly (such as between two heavy chains) or intramolecularly (such as between two cysteine residues in a single polypeptide chain). Generally and preferably, the disulfide linkage formed by the cysteine residue(s) that is altered (i.e., rendered incapable of forming disulfide linkages) is one that, when not present in an antibody, does not result in a substantial loss of the normal physicochemical and/or biological characteristics of the immunoglobulin. Preferably, but not necessarily, the cysteine residue that is rendered incapable of forming disulfide linkages is a cysteine of the hinge region of a heavy chain.

An antibody with variant heavy chains or variant hinge region is generally produced by expressing in a host cell an antibody in which at least one, at least two, at least three, at least four, or between two and eleven inter-heavy chain disulfide linkages are eliminated, and recovering said antibody from the host cell. Expression of said antibody can be from a polynucleotide encoding an antibody, said antibody comprising a variant heavy chain with reduced disulfide linkage capability, followed by recovering said antibody from the host cell comprising the polynucleotide. Preferably, said heavy chain comprises a variant hinge region of an immunoglobulin heavy chain, wherein at least one cysteine of said variant hinge region is rendered incapable of forming a disulfide linkage.

It is further anticipated that any cysteine in an immunoglobulin heavy chain can be rendered incapable of disulfide linkage formation, similarly to the hinge cysteines described herein, provided that such alteration does not substantially reduce the biological function of the immunoglobulin. For example, IgM and IgE lack a hinge region, but each contains an extra heavy chain domain; at least one (in some embodiments, all) of the cysteines of the heavy chain can be rendered incapable of disulfide linkage formation in methods of the invention so long as it does not substantially reduce the biological function of the heavy chain and/or the antibody which comprises the heavy chain.

Heavy chain hinge cysteines are well known in the art, as described, for example, in Kabat, 1991, “Sequences of proteins of immunological interest,” supra. As is known in the art, the number of hinge cysteines varies depending on the class and subclass of immunoglobulin. See, for example, Janeway, 1999, Immunobiology, 4th Ed., (Garland Publishing, NY). For example, in human IgGIs, two hinge cysteines are separated by two prolines, and these are normally paired with their counterparts on an adjacent heavy chain in intermolecular disulfide linkages. Other examples include human IgG2 that contains 4 hinge cysteines, IgG3 that contains 11 hinge cysteines, and IgG4 that contains 2 hinge cysteines.

Accordingly, methods of the invention include expressing in a host cell an immunoglobulin heavy chain comprising a variant hinge region, where at least one cysteine of the variant hinge region is rendered incapable of forming a disulfide linkage, allowing the heavy chain to complex with a light chain to form a biologically active antibody, and recovering the antibody from the host cell.

Alternative embodiments include those where at least 2, 3, or 4 cysteines are rendered incapable of forming a disulfide linkage; where from about two to about eleven cysteines are rendered incapable; and where all the cysteines of the variant hinge region are rendered incapable.

Light chains and heavy chains constituting antibodies of the invention as produced according to methods of the invention may be encoded by a single polynucleotide or by separate polynucleotides.

Cysteines normally involved in disulfide linkage formation can be rendered incapable of forming disulfide linkages by any of a variety of methods known in the art, or those that would be evident to one skilled in the art in view of the criteria described herein. For example, a hinge cysteine can be substituted with another amino acid, such as serine that is not capable of disulfide bonding. Amino acid substitution can be achieved by standard molecular biology techniques, such as site directed mutagenesis of the nucleic acid sequence encoding the hinge region that is to be modified. Suitable techniques include those described in Sambrook et al., 1989, Molecular Cloning: A Laboratory Manual, 2nd Ed., Other techniques for generating an immunoglobulin with a variant hinge region include synthesizing an oligonucleotide that encodes a hinge region, where the codon for the cysteine to be substituted is replaced with a codon for the substitute amino acid. This oligonucleotide can then be ligated into a vector backbone comprising other appropriate antibody sequences, such as variable regions and Fc sequences, as appropriate.

In another embodiment, a hinge cysteine can be deleted. Amino acid deletion can be achieved by standard molecular biology techniques, such as site directed mutagenesis of the nucleic acid sequence encoding the hinge region that is to be modified. Suitable techniques include those described in Sambrook et al., supra. Other techniques for generating an immunoglobulin with a variant hinge region include synthesizing an oligonucleotide comprising a sequence that encodes a hinge region in which the codon for the cysteine to be modified is deleted. This oligonucleotide can then be ligated into a vector backbone comprising other appropriate antibody sequences, such as variable regions and Fc sequences, as appropriate.

F. Bispecific Antibodies Formed Using “Protuberance-Into-Cavity” Strategy

In some embodiments, bispecific antibodies of the invention are formed using a “protuberance-into-cavity” strategy, also referred to as “knobs into holes” that serves to engineer an interface between a first and second polypeptide for hetero-oligomerization. The preferred interface comprises at least a part of the CH3 domain of an antibody constant domain. The “knobs into holes” mutations in the CH3 domain of an Fc sequence has been reported to greatly reduce the formation of homodimers (See, for example, Merchant et al., 1998, Nature Biotechnology, 16:677-681). “Protuberances” are constructed by replacing small amino acid side chains from the interface of the first polypeptide with larger side chains (e.g. tyrosine or tryptophan). Compensatory “cavities” of identical or similar size to the protuberances are optionally created on the interface of the second polypeptide by replacing large amino acid side chains with smaller ones (e.g. alanine or threonine). Where a suitably positioned and dimensioned protuberance or cavity exists at the interface of either the first or second polypeptide, it is only necessary to engineer a corresponding cavity or protuberance, respectively, at the adjacent interface. The protuberance and cavity can be made by synthetic means such as altering the nucleic acid encoding the polypeptides or by peptide synthesis. For further description of knobs into holes, see U.S. Pat. Nos. 5,731,168; 5,807,706; 5,821,333.

In some embodiments “knobs into holes” technology is used to promote heterodimerization to generate full-length bispecific anti-FcγRIIB and anti-“activating receptor” (e.g., IgER) antibody. In one embodiment, constructs were prepared for the anti-FcγIIB component (e.g., p5A6.11.Knob) by introducing the “knob” mutation (T366W) into the Fc region, and the anti-IgER component (e.g., p22E7.11.Hole) by introducing the “hole” mutations (T366S, L368A, Y407V). In another embodiment, constructs are prepared for the anti-FcγIIB component (e.g., p5A6.11.Hole) by introducing a “hole” mutation into its Fc region, and the anti-IgER component (e.g., p22E7.11.Knob) by introducing a “knob” mutation in its Fc region such as by the procedures disclosed herein or the procedures disclosed by Merchant et al., (1998), supra, or in U.S. Pat. Nos. 5,731,168; 5,807,706; 5,821,333.

A general method of preparing a heteromultimer using the “protuberance-into-cavity” strategy comprises expressing, in one or separate host cells, a polynucleotide encoding a first polypeptide that has been altered from an original polynucleotide to encode a protuberance, and a second polynucleotide encoding a second polypeptide that has been altered from the original polynucleotide to encode the cavity. The polypeptides are expressed, either in a common host cell with recovery of the heteromultimer from the host cell culture, or in separate host cells, with recovery and purification, followed by formation of the heteromultimer. In some embodiments, the heteromultimer formed is a multimeric antibody, for example a bispecific antibody. See also U.S. patent application Ser. No. 13/092,708 filed 22 Apr. 2011.

In some embodiments, antibodies of the present invention combine a knobs into holes strategy with variant hinge region constructs to produce hingeless bispecific antibodies.

G. Immunoconjugates

The invention also provides immunoconjugates comprising an anti-IL-1β antibody and/or anti-IL-18 antibody/antibodies herein conjugated to one or more cytotoxic agents, such as chemotherapeutic agents or drugs, growth inhibitory agents, toxins (e.g., protein toxins, enzymatically active toxins of bacterial, fungal, plant, or animal origin, or fragments thereof), or radioactive isotopes.

In one embodiment, an immunoconjugate is an antibody-drug conjugate (ADC) in which an antibody is conjugated to one or more drugs, including but not limited to a maytansinoid (see U.S. Pat. Nos. 5,208,020, 5,416,064 and European Patent EP 0 425 235 B1); an auristatin such as monomethylauristatin drug moieties DE and DF (MMAE and MMAF) (see U.S. Pat. Nos. 5,635,483 and 5,780,588, and 7,498,298); a dolastatin; a calicheamicin or derivative thereof (see U.S. Pat. Nos. 5,712,374, 5,714,586, 5,739,116, 5,767,285, 5,770,701, 5,770,710, 5,773,001, and 5,877,296; Hinman et al., Cancer Res. 53:3336-3342 (1993); and Lode et al., Cancer Res. 58:2925-2928 (1998)); an anthracycline such as daunomycin or doxorubicin (see Kratz et al., Current Med. Chem. 13:477-523 (2006); Jeffrey et al., Bioorganic & Med. Chem. Letters 16:358-362 (2006); Torgov et al., Bioconj. Chem. 16:717-721 (2005); Nagy et al., Proc. Natl. Acad. Sci. USA 97:829-834 (2000); Dubowchik et al., Bioorg. & Med. Chem. Letters 12:1529-1532 (2002); King et al., J. Med. Chem. 45:4336-4343 (2002); and U.S. Pat. No. 6,630,579); methotrexate; vindesine; a taxane such as docetaxel, paclitaxel, larotaxel, tesetaxel, and ortataxel; a trichothecene; and CC1065.

In another embodiment, an immunoconjugate comprises an antibody as described herein conjugated to an enzymatically active toxin or fragment thereof, including but not limited to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes.

In another embodiment, an immunoconjugate comprises an antibody as described herein conjugated to a radioactive atom to form a radioconjugate. A variety of radioactive isotopes are available for the production of radioconjugates. Examples include At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu. When the radioconjugate is used for detection, it may comprise a radioactive atom for scintigraphic studies, for example tc99m or I123, or a spin label for nuclear magnetic resonance (NMR) imaging (also known as magnetic resonance imaging, mri), such as iodine-123 again, iodine-131, indium-111, fluorine-19, carbon-13, nitrogen-15, oxygen-17, gadolinium, manganese or iron.

Conjugates of an antibody and cytotoxic agent may be made using a variety of bifunctional protein coupling agents such as N-succinimidyl-3-(2-pyridyldithio) propionate (SPDP), succinimidyl-4-(N-maleimidomethyl)cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science 238:1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026. The linker may be a “cleavable linker” facilitating release of a cytotoxic drug in the cell. For example, an acid-labile linker, peptidase-sensitive linker, photolabile linker, dimethyl linker or disulfide-containing linker (Chari et al., Cancer Res. 52:127-131 (1992); U.S. Pat. No. 5,208,020) may be used.

The immunuoconjugates or ADCs herein expressly contemplate, but are not limited to such conjugates prepared with cross-linker reagents including, but not limited to, BMPS, EMCS, GMBS, HBVS, LC-SMCC, MBS, MPBH, SBAP, SIA, STAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone)benzoate) which are commercially available (e.g., from Pierce Biotechnology, Inc., Rockford, Ill., U.S.A).

### II. Vectors, Host Cells and Recombinant Methods

The invention also provides isolated polynucleotides encoding the antibodies as disclosed herein, vectors and host cells comprising the polynucleotides, and recombinant techniques for the production of the antibodies.

For recombinant production of the antibody, a polynucleotide encoding the antibody is isolated and inserted into a replicable vector for further cloning (amplification of the DNA) or for expression. DNA encoding the antibody is readily isolated and sequenced using conventional procedures, for example, by using oligonucleotide probes capable of binding specifically to genes encoding the antibody. Many vectors are available. The vector components generally include, but are not limited to, one or more of the following: a signal sequence, an origin of replication, one or more marker genes, an enhancer element, a promoter, and a transcription termination sequence.

(i) Signal Sequence Component

The antibodies of this invention may be produced recombinantly, not only directly, but also as fusion antibodies with heterologous antibodies. In one embodiment, the heterologous antibody is a signal sequence or other antibody having a specific cleavage site at the N-terminus of the mature protein or antibody. The heterologous signal sequence selected preferably is one that is recognized and processed (i.e., cleaved by a signal peptidase) by the host cell. For prokaryotic host cells that do not recognize and process the native antibody signal sequence, the signal sequence is substituted by a prokaryotic signal sequence selected, for example, from the group of the alkaline phosphatase, penicillinase, 1 pp, or heat-stable enterotoxin II leaders. For yeast secretion the native signal sequence may be substituted by, e.g., the yeast invertase leader, a factor leader (including Saccharomyces and Kluyveromyces α-factor leaders), or acid phosphatase leader, the C. albicans glucoamylase leader, or the signal described in WO 90/13646. In mammalian cell expression, mammalian signal sequences as well as viral secretory leaders, for example, the herpes simplex gD signal, are available. The DNA for such precursor region is ligated in reading frame to DNA encoding the antibody.

In another embodiment, production of antibodies can occur in the cytoplasm of the host cell, and therefore does not require the presence of secretion signal sequences within each cistron. In that regard, immunoglobulin light and heavy chains are expressed, folded, and assembled to form functional immunoglobulins within the cytoplasm. Certain host strains (for example, the E. coli trxB strains) provide cytoplasm conditions that are favorable for disulfide bond formation, thereby permitting proper folding and assembly of expressed protein subunits (Proba and Plukthun, 1995, Gene, 159:203).

(ii) Origin of Replication Component

Both expression and cloning vectors contain a nucleic acid sequence that enables the vector to replicate in one or more selected host cells. Generally, in cloning vectors this sequence is one that enables the vector to replicate independently of the host chromosomal DNA, and includes origins of replication or autonomously replicating sequences. Such sequences are well known for a variety of bacteria, yeast, and viruses. The origin of replication from the plasmid pBR322 is suitable for most Gram-negative bacteria, the 2 μplasmid origin is suitable for yeast, and various viral origins (SV40, polyoma, adenovirus, VSV, or BPV) are useful for cloning vectors in mammalian cells. Generally, the origin of replication component is not needed for mammalian expression vectors (the SV40 origin may typically be used only because it contains the early promoter).

(iii) Selection Gene Component

Expression and cloning vectors may contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli.

One example of a selection scheme utilizes a drug to arrest growth of a host cell. Those cells that are successfully transformed with a heterologous gene produce a protein conferring drug resistance and thus survive the selection regimen. Examples of such dominant selection use the drugs neomycin, mycophenolic acid and hygromycin.

Another example of suitable selectable markers for mammalian cells are those that enable the identification of cells competent to take up the antibody nucleic acid, such as DHFR, thymidine kinase, metallothionein-I and -II, preferably primate metallothionein genes, adenosine deaminase, ornithine decarboxylase, and the like.

For example, cells transformed with the DHFR selection gene are first identified by culturing all of the transformants in a culture medium that contains methotrexate (Mtx), a competitive antagonist of DHFR. An appropriate host cell when wild-type DHFR is employed is the Chinese hamster ovary (CHO) cell line deficient in DHFR activity.

Alternatively, host cells (particularly wild-type hosts that contain endogenous DHFR) transformed or co-transformed with DNA sequences encoding antibody, wild-type DHFR protein, and another selectable marker such as aminoglycoside 3′-phosphotransferase (APH) can be selected by cell growth in medium containing a selection agent for the selectable marker such as an aminoglycosidic antibiotic, e.g., kanamycin, neomycin, or G418. See U.S. Pat. No. 4,965,199.

A suitable selection gene for use in yeast is the trp 1 gene present in the yeast plasmid YRp7 (Stinchcomb et al., 1979, Nature, 282:39). The trp1 gene provides a selection marker for a mutant strain of yeast lacking the ability to grow in tryptophan, for example, ATCC No. 44076 or PEP4-1. Jones, 1977, Genetics, 85:12. The presence of the trp1 lesion in the yeast host cell genome then provides an effective environment for detecting transformation by growth in the absence of tryptophan. Similarly, Leu2-deficient yeast strains (for example, strains having ATCC accession number 20,622 or 38,626) are complemented by known plasmids bearing the Leu2 gene.

In addition, vectors derived from the 1.6 μm circular plasmid pKD1 can be used for transformation of Kluyveromyces yeasts. Alternatively, an expression system for large-scale production of recombinant calf chymosin was reported for K. lactis. See Van den Berg, 1990, Bio/Technology, 8:135. Stable multi-copy expression vectors for secretion of mature recombinant human serum albumin by industrial strains of Kluyveromyces have also been disclosed. See Fleer et al., 1991, Bio/Technology, 9:968-975.

(iv) Promoter Component

Expression and cloning vectors usually contain a promoter that is recognized by the host organism and is operably linked to the antibody nucleic acid. Promoters suitable for use with prokaryotic hosts include the phoA promoter, β-lactamase and lactose promoter systems, alkaline phosphatase, a tryptophan (trp) promoter system, and hybrid promoters such as the tac promoter. However, other known bacterial promoters are suitable. Promoters for use in bacterial systems also will contain a Shine-Dalgarno (S.D.) sequence operably linked to the DNA encoding the antibody.

Promoter sequences are known for eukaryotes. Virtually all eukaryotic genes have an AT-rich region located approximately 25 to 30 bases upstream from the site where transcription is initiated. Another sequence found 70 to 80 bases upstream from the start of transcription of many genes is a CNCAAT region where N may be any nucleotide. At the 3′ end of most eukaryotic genes is an AATAAA sequence that may be the signal for addition of the poly A tail to the 3′ end of the coding sequence. All of these sequences are suitably inserted into eukaryotic expression vectors.

Examples of suitable promoting sequences for use with yeast hosts include the promoters for 3-phosphoglycerate kinase or other glycolytic enzymes, such as enolase, glyceraldehyde-3-phos-phate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase.

Other yeast promoters, which are inducible promoters having the additional advantage of transcription controlled by growth conditions, are the promoter regions for alcohol dehydrogenase 2, isocytochrome C, acid phosphatase, degradative enzymes associated with nitrogen metabolism, metallothionein, glyceraldehyde-3-phosphate dehydrogenase, and enzymes responsible for maltose and galactose utilization. Suitable vectors and promoters for use in yeast expression are further described in EP 73,657. Yeast enhancers also are advantageously used with yeast promoters.

Antibody transcription from vectors in mammalian host cells is controlled, for example, by promoters obtained from the genomes of viruses such as polyoma virus, fowlpox virus, adenovirus (such as Adenovirus 2), bovine papilloma virus, avian sarcoma virus, cytomegalovirus, a retrovirus, hepatitis-B virus and most preferably Simian Virus 40 (SV40), from heterologous mammalian promoters, e.g., the actin promoter or an immunoglobulin promoter, from heat-shock promoters, provided such promoters are compatible with the host cell systems.

The early and late promoters of the SV40 virus are conveniently obtained as an SV40 restriction fragment that also contains the SV40 viral origin of replication. The immediate early promoter of the human cytomegalovirus is conveniently obtained as a HindIII E restriction fragment. A system for expressing DNA in mammalian hosts using the bovine papilloma virus as a vector is disclosed in U.S. Pat. No. 4,419,446. A modification of this system is described in U.S. Pat. No. 4,601,978. See also Reyes et al., 1982, Nature 297:598-601 on expression of human β-interferon cDNA in mouse cells under the control of a thymidine kinase promoter from herpes simplex virus. Alternatively, the rous sarcoma virus long terminal repeat can be used as the promoter.

(v) Enhancer Element Component

Transcription of a DNA encoding the antibody of this invention by higher eukaryotes is often increased by inserting an enhancer sequence into the vector. Many enhancer sequences are now known from mammalian genes (globin, elastase, albumin, a-fetoprotein, and insulin). Typically, however, one will use an enhancer from a eukaryotic cell virus. Examples include the SV40 enhancer on the late side of the replication origin (bp 100-270), the cytomegalovirus early promoter enhancer, the polyoma enhancer on the late side of the replication origin, and adenovirus enhancers. See also Yaniv, 1982, Nature 297:17-18 on enhancing elements for activation of eukaryotic promoters. The enhancer may be spliced into the vector at a position 5′ or 3′ to the antibody-encoding sequence, but is preferably located at a site 5′ from the promoter.

(vi) Transcription Termination Component

Expression vectors used in eukaryotic host cells (yeast, fungi, insect, plant, animal, human, or nucleated cells from other multicellular organisms) will also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the mRNA encoding the antibody. One useful transcription termination component is the bovine growth hormone polyadenylation region. See WO94/11026 and the expression vector disclosed therein.

(vii) Modulation of Translational Strength

Immunoglobulins of the present invention can also be expressed from an expression system in which the quantitative ratio of expressed light and heavy chains can be modulated in order to maximize the yield of secreted and properly assembled full length antibodies. Such modulation is accomplished by simultaneously modulating translational strengths for light and heavy chains.

One technique for modulating translational strength is disclosed in Simmons et al., U.S. Pat. No. 5,840,523 and Simmons et al., 2002, J. Immunol. Methods, 263: 133-147. It utilizes variants of the translational initiation region (TIR) within a cistron. For a given TIR, a series of amino acid or nucleic acid sequence variants can be created with a range of translational strengths, thereby providing a convenient means by which to adjust this factor for the desired expression level of the specific chain. TIR variants can be generated by conventional mutagenesis techniques that result in codon changes which can alter the amino acid sequence, although silent changes in the nucleotide sequence are preferred. Alterations in the TIR can include, for example, alterations in the number or spacing of Shine-Dalgamo sequences, along with alterations in the signal sequence. One preferred method for generating mutant signal sequences is the generation of a “codon bank” at the beginning of a coding sequence that does not change the amino acid sequence of the signal sequence (i.e., the changes are silent). This can be accomplished by changing the third nucleotide position of each codon; additionally, some amino acids, such as leucine, serine, and arginine, have multiple first and second positions that can add complexity in making the bank. This method of mutagenesis is described in detail in Yansura et al, 1992, METHODS: A Companion to Methods in Enzymol., 4:151-158.

Preferably, a set of vectors is generated with a range of TIR strengths for each cistron therein. This limited set provides a comparison of expression levels of each chain as well as the yield of full length products under various TIR strength combinations. TIR strengths can be determined by quantifying the expression level of a reporter gene as described in detail in Simmons et al., U.S. Pat. No. 5,840,523 and Simmons et al., 2002, J. Immunol. Methods, 263: 133-147. For the purpose of this invention, the translational strength combination for a particular pair of TIRs within a vector is represented by (N-light, M-heavy), wherein N is the relative TIR strength of light chain and M is the relative TIR strength of heavy chain. For example, (3-light, 7-heavy) means the vector provides a relative TIR strength of about 3 for light chain expression and a relative TIR strength of about 7 for heavy chain expression. Based on the translational strength comparison, the desired individual TIRs are selected to be combined in the expression vector constructs of the invention.

(viii) Selection and Transformation of Host Cells

Suitable host cells for cloning or expressing the DNA in the vectors herein are the prokaryote, yeast, or higher eukaryote cells described above. Suitable prokaryotes for this purpose include Archaebacteria and Eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis (e.g., B. licheniformis 41P disclosed in DD 266,710, published 12 Apr. 1989), Pseudomonas such as P. aeruginosa, and Streptomyces. One preferred E. coli cloning host is E. coli 294 (ATCC 31,446), although other strains such as E. coli B, E. coli X1776 (ATCC 31,537), and E. coli W3110 (ATCC 27,325) are suitable. These examples are illustrative rather than limiting. It is also preferably for the host cell to secrete minimal amounts of proteolytic enzymes, and additional protease inhibitors may desirably be incorporated in the cell culture. Prokaryotic host cells may also comprise mutation(s) in the thioredoxin and/or glutathione pathways.

In addition to prokaryotes, eukaryotic microbes such as filamentous fungi or yeast are suitable cloning or expression hosts for antibody-encoding vectors. Saccharomyces cerevisiae, or common baker's yeast, is the most commonly used among lower eukaryotic host microorganisms. However, a number of other genera, species, and strains are commonly available and useful herein, such as Schizosaccharomyces pombe; Kluyveromyces hosts such as, e.g., K. lactis, K. fragilis (ATCC 12,424), K. bulgaricus (ATCC 16,045), K. wickeramii (ATCC 24,178), K. waltii (ATCC 56,500), K. drosophilarum (ATCC 36,906), K. thermotolerans, and K. marxianus; yarrowia (EP 402,226); Pichia pastoris (EP 183,070); Candida; Trichoderma reesia (EP 244,234); Neurospora crassa; Schwanniomyces such as Schwanniomyces occidentalis; and filamentous fungi such as, e.g., Neurospora, Penicillium, Tolypocladium, and Aspergillus hosts such as A. nidulans and A. niger.

Suitable host cells for the expression of glycosylated antibody are derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori have been identified. A variety of viral strains for transfection are publicly available, e.g., the L-1 variant of Autographa californica NPV and the Bm-5 strain of Bombyx mori NPV, and such viruses may be used as the virus herein according to the present invention, particularly for transfection of Spodoptera frugiperda cells. Plant cell cultures of cotton, corn, potato, soybean, petunia, tomato, and tobacco can also be utilized as hosts.

Vertebrate host cells are widely used, and propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/−DHFR (CHO, Urlaub et al., 1980, Proc. Natl. Acad. Sci. USA 77:4216); mouse sertoli cells (TM4, Mather, 1980, Biol. Reprod. 23:243-251); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., 1982, Annals N.Y. Acad. Sci. 383:44-68); MRC 5 cells; FS4 cells; mouse myeloma cells, such as NSO (e.g. RCB0213, 1992, Bio/Technology 10:169) and SP2/0 cells (e.g. SP2/0-Ag14 cells, ATCC CRL 1581); rat myeloma cells, such as YB2/0 cells (e.g. YB2/3HL.P2.G11.16Ag.20 cells, ATCC CRL 1662); and a human hepatoma line (Hep G2). CHO cells are a preferred cell line for practicing the invention, with CHO-K1, DUK-B11, CHO-DP12, CHO-DG44 (Somatic Cell and Molecular Genetics 12:555 (1986)), and Lec13 being exemplary host cell lines. In the case of CHO-K1, DUK-B11, DG44 or CHO-DP12 host cells, these may be altered such that they are deficient in their ability to fucosylate proteins expressed therein.

The invention is also applicable to hybridoma cells. The term “hybridoma” refers to a hybrid cell line produced by the fusion of an immortal cell line of immunologic origin and an antibody producing cell. The term encompasses progeny of heterohybrid myeloma fusions, which are the result of a fusion with human cells and a murine myeloma cell line subsequently fused with a plasma cell, commonly known as a trioma cell line. Furthermore, the term is meant to include any immortalized hybrid cell line that produces antibodies such as, for example, quadromas (See, for example, Milstein et al., 1983, Nature, 537:3053). The hybrid cell lines can be of any species, including human and mouse.

In a most preferred embodiment the mammalian cell is a non-hybridoma mammalian cell, which has been transformed with exogenous isolated nucleic acid encoding the antibody of interest. By “exogenous nucleic acid” or “heterologous nucleic acid” is meant a nucleic acid sequence that is foreign to the cell, or homologous to the cell but in a position within the host cell nucleic acid in which the nucleic acid is ordinarily not found.

(ix) Culturing the Host Cells

Host cells are transformed with the above-described expression or cloning vectors for antibody production and cultured in conventional nutrient media modified as appropriate for inducing promoters, selecting transformants, or amplifying the genes encoding the desired sequences.

The host cells used to produce the antibody of this invention may be cultured in a variety of media. Commercially available media such as Ham's F10 (Sigma), Minimal Essential Medium ((MEM), (Sigma), RPMI-1640 (Sigma)), and Dulbecco's Modified Eagle's Medium ((DMEM), Sigma) are suitable for culturing the host cells. In addition, any of the media described in Ham et al., 1979, Meth. Enz. 58:44, Barnes et al., 1980, Anal. Biochem. 102:255, U.S. Pat. No. 4,767,704; 4,657,866; 4,927,762; 4,560,655; or 5,122,469; WO 90/03430; WO 87/00195; or U.S. Pat. Re. No. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

All culture medium typically provides at least one component from one or more of the following categories:

1) an energy source, usually in the form of a carbohydrate such as glucose;

2) all essential amino acids, and usually the basic set of twenty amino acids plus cystine;

3) vitamins and/or other organic compounds required at low concentrations;

4) free fatty acids; and

5) trace elements, where trace elements are defined as inorganic compounds or naturally occurring elements that are typically required at very low concentrations, usually in the micromolar range.

The culture medium is preferably free of serum, e.g. less than about 5%, preferably less than 1%, more preferably 0 to 0.1% serum, and other animal-derived proteins. However, they can be used if desired. In a preferred embodiment of the invention the cell culture medium comprises excess amino acids. The amino acids that are provided in excess may, for example, be selected from Asn, Asp, Gly, Ile, Leu, Lys, Met, Ser, Thr, Trp, Tyr, and Val. Preferably, Asn, Asp, Lys, Met, Ser, and Trp are provided in excess. For example, amino acids, vitamins, trace elements and other media components at one or two times the ranges specified in European Patent EP 307,247 or U.S. Pat. No. 6,180,401 may be used. These two documents are incorporated by reference herein.

For the culture of the mammalian cells expressing the desired protein and capable of adding the desired carbohydrates at specific positions, numerous culture conditions can be used paying particular attention to the host cell being cultured. Suitable culture conditions for mammalian cells are well known in the art (W. Louis Cleveland et al., 1983, J. Immunol. Methods 56:221-234) or can be easily determined by the skilled artisan (see, for example, Animal Cell Culture: A Practical Approach 2^{nd} Ed., Rickwood, D. and Hames, B. D., eds. Oxford University Press, New York (1992)), and vary according to the particular host cell selected.

(x) Antibody Purification

When using recombinant techniques, the antibody can be produced intracellularly, in the periplasmic space, or directly secreted into the medium. If the antibody is produced intracellularly, as a first step, the particulate debris, either host cells or lysed fragments, is removed, for example, by centrifugation or ultrafiltration. Carter et al., 1992, Bio/Technology 10: 163-167 describe a procedure for isolating antibodies which are secreted to the periplasmic space of E. coli. Briefly, cell paste is thawed in the presence of sodium acetate (pH 3.5), EDTA, and phenylmethylsulfonylfluoride (PMSF) over about 30 min. Cell debris can be removed by centrifugation. Where the antibody is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The antibody composition prepared from the cells can be purified using, for example, hydroxylapatite chromatography, gel electrophoresis, dialysis, and affinity chromatography, with affinity chromatography being the preferred purification technique. The suitability of protein A as an affinity ligand depends on the species and isotype of any immunoglobulin Fc region that is present in the antibody. Protein A can be used to purify antibodies that are based on human γ1, γ2, or γ4 heavy chains (Lindmark et al., 1983, J. Immunol. Meth. 62:1-13). Protein G is recommended for all mouse isotypes and for human γ3 (Guss et al., 1986, EMBO J. 5:15671575). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a C_{H}3 domain, the Bakerbond ABX™ resin (J. T. Baker, Phillipsburg, N.J.) is useful for purification. Other techniques for protein purification such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the antibody to be recovered.

In one embodiment, the glycoprotein may be purified using adsorption onto a lectin substrate (e.g. a lectin affinity column) to remove fucose-containing glycoprotein from the preparation and thereby enrich for fucose-free glycoprotein.

(xi) Antibody Activity Assays

The immunoglobulins of the present invention can be characterized for their physical/chemical properties and biological functions by various assays known in the art. In one aspect of the invention, it is important to compare the selectivity of an antibody of the present invention to bind the immunogen versus other binding targets.

In certain embodiments of the invention, the immunoglobulins produced herein are analyzed for their biological activity. In some embodiments, the immunoglobulins of the present invention are tested for their antigen binding activity. The antigen binding assays that are known in the art and can be used herein include without limitation any direct or competitive binding assays using techniques such as western blots, radioimmunoassays, ELISA (enzyme linked immunosorbent assay), “sandwich” immunoassays, immunoprecipitation assays, fluorescent immunoassays, and protein A immunoassays. Illustrative antigen binding assays are provided below in the Examples section.

The purified immunoglobulins can be further characterized by a series of assays including, but not limited to, N-terminal sequencing, amino acid analysis, non-denaturing size exclusion high pressure liquid chromatography (HPLC), mass spectrometry, ion exchange chromatography, and papain digestion. Methods for protein quantification are well known in the art. For example, samples of the expressed proteins can be compared for their quantitative intensities on a Coomassie-stained SDS-PAGE. Alternatively, the specific band(s) of interest (e.g., the full length band) can be detected by, for example, western blot gel analysis and/or AME5-RP assay.

### III. Pharmaceutical Formulations

Therapeutic formulations of the antibody/antibodies can be prepared by mixing the antibody having the desired degree of purity with optional physiologically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) antibody; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG). Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

Exemplary lyophilized antibody formulations are described in U.S. Pat. No. 6,267,958. Aqueous antibody formulations include those described in U.S. Pat. No. 6,171,586 and WO2006/044908, the latter formulations including a histidine-acetate buffer.

The formulation herein may also contain more than one active compound as necessary for the particular indication being treated, preferably those with complementary activities that do not adversely affect each other. For instance, the formulation may further comprise another antibody or a chemotherapeutic agent. Such molecules are suitably present in combination in amounts that are effective for the purpose intended.

The active ingredients may also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

The formulations to be used for in vivo administration must be sterile. This is readily accomplished by filtration through sterile filtration membranes.

Sustained-release preparations may be prepared. Suitable examples of sustained-release preparations include semipermeable matrices of solid hydrophobic polymers containing the antibody, which matrices are in the form of shaped articles, e.g., films, or microcapsule. Examples of sustained-releabe matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated antibodies remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

### IV. Non-Therapeutic Uses for the Antibody

The antibody of the invention may be used as an affinity purification agent. In this process, the antibody is immobilized on a solid phase such a Sephadex™ resin or filter paper, using methods well known in the art. The immobilized antibody is contacted with a sample containing the antigen to be purified, and thereafter the support is washed with a suitable solvent that will remove substantially all the material in the sample except the antigen to be purified, which is bound to the immobilized antibody. Finally, the support is washed with another suitable solvent, such as glycine buffer, pH 5.0, that will release the antigen from the antibody.

The antibody may also be useful in diagnostic assays, e.g., for detecting expression of an antigen of interest in specific cells, tissues, or serum. For diagnostic applications, the antibody typically will be labeled with a detectable moiety. Numerous labels are available which can be generally grouped into the following categories:

(a) Radioisotopes, such as ³⁵S, ¹⁴C, ¹²⁵I, ³H, and ¹³¹I. The antibody can be labeled with the radioisotope using the techniques described in Current Protocols in Immunology, Volumes 1 and 2, Coligen et al., Ed. Wiley-Interscience, New York, N.Y., Pubs. (1991), for example, and radioactivity can be measured using scintillation counting.

(b) Fluorescent labels such as rare earth chelates (europium chelates) or fluorescein and its derivatives, rhodamine and its derivatives, dansyl, Lissamine, phycoerythrin and Texas Red are available. The fluorescent labels can be conjugated to the antibody using the techniques disclosed in Current Protocols in Immunology, supra, for example. Fluorescence can be quantified using a fluorimeter.

(c) Various enzyme-substrate labels are available and U.S. Pat. No. 4,275,149 provides a review of some of these. The enzyme generally catalyzes a chemical alteration of the chromogenic substrate that can be measured using various techniques. For example, the enzyme may catalyze a color change in a substrate, which can be measured spectrophotometrically. Alternatively, the enzyme may alter the fluorescence or chemiluminescence of the substrate. Techniques for quantifying a change in fluorescence are described above. The chemiluminescent substrate becomes electronically excited by a chemical reaction and may then emit light that can be measured (using a chemiluminometer, for example) or donates energy to a fluorescent acceptor. Examples of enzymatic labels include luciferases (e.g., firefly luciferase and bacterial luciferase; U.S. Pat. No. 4,737,456), luciferin, 2,3-dihydrophthalazinediones, malate dehydrogenase, urease, peroxidase such as horseradish peroxidase (HRPO), alkaline phosphatase, β-galactosidase, glucoamylase, lysozyme, saccharide oxidases (e.g., glucose oxidase, galactose oxidase, and glucose-6-phosphate dehydrogenase), heterocyclic oxidases (such as uricase and xanthine oxidase), lactoperoxidase, microperoxidase, and the like. Techniques for conjugating enzymes to antibodies are described in O'Sullivan et al., Methods for the Preparation of Enzyme-Antibody Conjugates for use in Enzyme Immunoassay, in Methods in Enzym. (ed J. Langone and H. Van Vunakis), Academic press, New York, 73:147-166 (1981).

Examples of enzyme-substrate combinations include, for example:

1) Horseradish peroxidase (HRPO) utilizes hydrogen peroxide to oxidize a dye precursor (e.g., orthophenylene diamine (OPD) or 3,3′,5,5′-tetramethyl benzidine hydrochloride (TMB));

2) alkaline phosphatase (AP) with para-Nitrophenyl phosphate as chromogenic substrate; and

3) β-D-galactosidase (β-D-Gal) with a chromogenic substrate (e.g., p-nitrophenyl-β-D-galactosidase) or fluorogenic substrate 4-methylumbelliferyl-β-D-galactosidase.

Numerous other enzyme-substrate combinations are available to those skilled in the art. For a general review of these, see U.S. Pat. Nos. 4,275,149 and 4,318,980.

Sometimes, the label is indirectly conjugated with the antibody. The skilled artisan will be aware of various techniques for achieving this. For example, the antibody can be conjugated with biotin and any of the three broad categories of labels mentioned above can be conjugated with avidin, or vice versa. Biotin binds selectively to avidin and thus, the label can be conjugated with the antibody in this indirect manner. Alternatively, to achieve indirect conjugation of the label with the antibody, the antibody is conjugated with a small hapten (e.g., digoxin) and one of the different types of labels mentioned above is conjugated with an anti-hapten antibody (e.g., anti-digoxin antibody). Thus, indirect conjugation of the label with the antibody can be achieved.

In another embodiment of the invention, the antibody need not be labeled, and the presence thereof can be detected using a labeled antibody which binds to the antibody.

The antibody of the present invention may be employed in any known assay method, such as competitive binding assays, direct and indirect sandwich assays, and immunoprecipitation assays. Zola, Monoclonal Antibodies: A Manual of Techniques, pp. 47-158 (CRC Press, Inc. 1987).

The antibody may also be used for in vivo diagnostic assays. Generally, the antibody is labeled with a radionuclide (such as ¹¹¹In, ⁹⁹Tc, ¹⁴C, ¹³¹I, ¹²⁵I, ³H, ³²P or ³⁵S) so that the antigen or cells expressing it can be localized using immunoscintiography.

### V. In Vivo Uses for the Antibody

In another embodiment, the anti-IL-1β and/or anti-IL-18 antibody/antibodies of the present invention is co-administered with a therapeutic agent to enhance the function of the therapeutic agent. For example, anti-FcγRIIB is administered to a mammal to block IgG binding to FcγRIIB, thereby preventing FcγRIIB-mediated inhibition of an immune response. This results in enhanced cytoxicity of an IgG therapeutic antibody. For example, where a therapeutic antibody is specific for a tumor antigen, co-administration of anti-FcγRIIB of the invention with the anti-tumor antigen antibody enhances cytoxicity of the anti-tumor antigen antibody.

Therapeutic antibodies, a number of which are described above, have been developed and approved for treatment of a variety of diseases, including cancer. For example, RITUXAN® (Rituximab) (IDEC Pharm/Genentech, Inc.) is used to treat B cell lymphomas, AVASTIN™ (bevacizumab) (Genentech, Inc.) is used to treat metastatic colorectal cancer and HERCEPTIN® (Trastumab) (Genentech, Inc.) is a humanized anti-HER2 monoclonal antibody used to treat metastatic breast cancer. Although, the mechanisms for treatment of cancer by all monoclonal antibodies developed for such treatment may not be completely understood, at least in some cases, a portion of the effectiveness of antibody therapy can be attributed to the recruitment of immune effector function (Houghton et al., 2000, Nature Medicine, 6:373-374; Clynes et al., 2000, Nature Medicine, 6:433-446). XOLAIR® (Omalizumab) (Genentech, Inc.) is an anti-IgE antibody used to treat allergies.

The therapeutic potential for such a bifunctional antibody would include attenuation of signals involved in inflammation and/or allergy. For example, when activated by IgE and allergen (via the FcFR), mast cells and basophils secrete inflammatory mediators and cytokines that act on vascular and muscular cells and recruit inflammatory cells. The inflammatory cells in turn secrete inflammatory mediators and recruit inflammatory cells, in a continuing process resulting in long-lasting inflammation. Consequently, means of controlling IgE induced mast cell activation provides a therapeutic approach to treating allergic diseases by interrupting the initiation of the inflammatory response. As described above, a bifunctional antibody comprises an antibody, or fragment thereof that selectively binds IL-1β and comprising an antibody, or fragment thereof, that selectively binds IL-18.

Additional bifunctional antibody examples (e.g., bispecific antibodies) comprise combinations of an antibody or fragment thereof that selectively binds IL-1β, and a second antibody or fragment thereof, that selectively binds IL-18. In some embodiments, the antibody of the present invention is used to activate inhibitory FcγRIIB receptors in a mammal treated with the antibody so as to inhibit pro-inflammatory signals and/or B cell activation mediated by activating receptors. Hence, the antibody is used to treat inflammatory disorders and/or autoimmune diseases such as those identified above.

For the prevention or treatment of disease, the appropriate dosage of antibody will depend on the type of disease to be treated, the severity and course of the disease, whether the antibody is administered for preventive or therapeutic purposes, previous therapy, the patient's clinical history and response to the antibody, and the discretion of the attending physician. The antibody is suitably administered to the patient at one time or over a series of treatments.

Depending on the type and severity of the disease, about 1 μg/kg to 15 mg/kg (e.g., 0.1-20 mg/kg) of antibody is an initial candidate dosage for administration to the patient, whether, for example, by one or more separate administrations, or by continuous infusion. A typical daily dosage might range from about 1 μg/kg to 100 mg/kg or more, depending on the factors mentioned above. For repeated administrations over several days or longer, depending on the condition, the treatment is sustained until a desired suppression of disease symptoms occurs. However, other dosage regimens may be useful. The progress of this therapy is easily monitored by conventional techniques and assays.

The antibody composition should be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The “therapeutically effective amount” of the antibody to be administered will be governed by such considerations, and is the minimum amount necessary to prevent, ameliorate, or treat a disease or disorder. The antibody need not be, but is optionally formulated with one or more agents currently used to prevent or treat the disorder in question. The effective amount of such other agents depends on the amount of antibody present in the formulation, the type of disorder or treatment, and other factors discussed above. These are generally used in the same dosages and with administration routes as used hereinbefore or about from 1 to 99% of the heretofore employed dosages.

For example, for treating autoimmune diseases where there is the involvement of an inflammatory cell (e.g., leukocyte) adhesion, migration and activation, such as rheumatoid arthritis and lupus, the antibody herein can be co-administered with, e.g., anti-LFA-1 antibody (such as an anti-CD11a or anti-CD18 antibody) or an anti-ICAM antibody such as ICAM-1, -2, or -3. Additional agents for treating rheumatoid arthritis in combination with the antibody herein include Enbrel™, DMARDS, e.g., methotrexate, and NSAIDs (non-steroidal anti-inflammatory drugs). More than one of such other active agents than the antibody herein may also be employed. Additionally, insulin can be used for treating diabetes, anti-IgE for asthma, anti-CD11a for psoriasis, anti-alpha4beta7 and growth hormone (GH) for inflammatory bowel disease.

Furthermore, the formulation is suitably administered along with an effective amount of a hypoglycemic agent. For purposes herein, the term “hypoglycemic agent” refers to compounds that are useful for regulating glucose metabolism, preferably oral agents. More preferred herein for human use are insulin and the sulfonylurea class of oral hypoglycemic agents, which cause the secretion of insulin by the pancreas. Examples include glyburide, glipizide, and gliclazide. In addition, agents that enhance insulin sensitivity or are insulin sensitizing, such as biguanides (including metformin and phenformin) and thiazolidenediones such as REZULIN™ (troglitazone) brand insulin-sensitizing agent, and other compounds that bind to the PPAR-gamma nuclear receptor, are within this definition, and also are preferred.

The hypoglycemic agent is administered to the mammal by any suitable technique including parenterally, intranasally, orally, or by any other effective route. Most preferably, the administration is by the oral route. For example, MICRONASE™ tablets (glyburide) marketed by Upjohn in 1.25, 2.5, and 5 mg tablet concentrations are suitable for oral administration. The usual maintenance dose for Type II diabetics, placed on this therapy, is generally in the range of from or about 1.25 to 20 mg per day, which may be given as a single dose or divided throughout the day as deemed appropriate. Physician's Desk Reference, 2563-2565 (1995). Other examples of glyburide-based tablets available for prescription include GLYNASE™ brand drug (Upjohn) and DIABETA™ brand drug (Hoechst-Roussel). GLUCOTROL™ (Pratt) is the trademark for a glipizide (1-cyclohexyl-3-(p-(2-(5-methylpyrazine carboxamide)ethyl)phenyl)sulfonyl)urea) tablet available in both 5- and 10-mg strengths and is also prescribed to Type II diabetics who require hypoglycemic therapy following dietary control or in patients who have ceased to respond to other sulfonylureas. Physician's Desk Reference, 1902-1903 (1995). Other hypoglycemic agents than sulfonylureas, such as the biguanides (e.g., metformin and phenformin) or thiazolidinediones (e.g., troglitozone), or other drugs affecting insulin action may also be employed. If a thiazolidinedione is employed with the peptide, it is used at the same level as currently used or at somewhat lower levels, which can be adjusted for effects seen with the peptide alone or together with the dione. The typical dose of troglitazone (REZULIN™) employed by itself is about 100-1000 mg per day, more preferably 200-800 mg/day, and this range is applicable herein. See, for example, Ghazzi et al., Diabetes, 46: 433-439 (1997). Other thiazolidinediones that are stronger insulin-sensitizing agents than troglitazone would be employed in lower doses.

### VI. Articles of Manufacture

In another aspect of the invention, an article of manufacture containing materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). At least one active agent in the composition is an antibody of the invention. The label or package insert indicates that the composition is used for treating the condition of choice. Moreover, the article of manufacture may comprise (a) a first container with a composition contained therein, wherein the composition comprises an antibody of the invention; and (b) a second container with a composition contained therein, wherein the composition comprises a further cytotoxic or otherwise therapeutic agent. The article of manufacture in this embodiment of the invention may further comprise a package insert indicating that the compositions can be used to treat a particular condition. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

Therapeutic antibody compositions generally are placed into a container having a sterile access port, for example, an intravenous solution bag or vial having a stopper pierceable by a hypodermic injection needle.

The invention further provides an article of manufacture and kit containing materials useful for the treatment of cancer or a disease, for example. The article of manufacture comprises a container with a label. Suitable containers include, for example, bottles, vials, and test tubes. The containers may be formed from a variety of materials such as glass or plastic. The container holds a composition comprising the antibody described herein. The active agent in the composition is the particular antibody. The label on the container indicates that the composition is used for the treatment or prevention of a particular disease or disorder, and may also indicate directions for in vivo, such as those described above.

The kit of the invention comprises the container described above and a second container comprising a buffer. It may further include other materials desirable from a commercial and user standpoint, including other buffers, diluents, filters, needles, syringes, and package inserts with instructions for use.

It is understood that any of the above articles of manufacture may include an immunoconjugate of the invention in place of or in addition to an IL-1β and/or IL-18 antibody/antibodies.

### EXAMPLES

The following are examples of methods and compositions of the invention, and are provided herein for illustrative purposes, and are not intended to limit the scope of the present invention. It is understood that various other embodiments may be practiced, given the general description provided herein. The disclosures of all patent and scientific literatures cited herein are expressly incorporated in their entirety by reference.

Commercially available reagents referred to in the examples were used according to manufacturer's instructions unless otherwise indicated. The source of those cells identified in the following examples, and throughout the specification, by ATCC accession numbers is the American Type Culture Collection, Manassas, Va.

### Methods

### Dextran Sodium Sulfate (DSS)-Induced Colitis

Age and sex-matched wild-type and knock-out mice between 19-25 grams in weight will remain untreated or receive 3.5% DSS in their drinking water ad lib for 5 days. The mice will be scored daily for weight loss starting on day 4 and sacrificed on day 8. The colons will then be collected, scored, and used for organ culture or histopathology. Scoring of colons for degree of inflammation was done as follows: After removal of the feces by extensive flushing, the colons are scored based on the extent of wall thickening. The score ranges from 0 to 4, with normal colons scored as 0 and colons with thickened wall covering the entire length as 4.

### Colon Culture for Cytokine Profiling

Colons from mice are cleaned, opened longitudinally, and placed in RPMI medium containing 1% penicillin/streptomycin solution. After overnight incubation at 37° C., the culture medium is collected and clarified prior to cytokine analysis by xMAP-based technology (Luminex) using BioRad Bio-Plex single or 23-plex assays.

### AAV2/5 Subretinal Injection

Animals were anesthetized by intraperitoneal injection of ketamine/xylazine (80 mg/kg:15 mg/kg). Under a dissecting microscope, a 30 g insulin needle was used to create a puncture through the sclera, allowing for subretinal injection of 1 μl of 1×10¹² AAV2/5 genomic particles/ml (Genedetect, Bradenton, Fla.) using a 33-gauge Hamilton needle and a micro-auto-injector (World Precision Instruments, Sarasota, Fla.). Formation of a subretinal bubble indicated a successful injection.

### Western Blots

Ten week old Balbc mice were injected subretinally with 1 μl of 1×10⁹ AAV2/5-IL1β or AAV2/5 empty control virus (Genedetect). After 4 months infection, some mice underwent intense light exposure (ILE, 8000 lux) for 3.5 hours then placed in the dark for 48 hours. Eyes were dissected and eye cups (eye minus cornea and lens) were minced in cell lysis buffer (Cell Signaling Technologies, Danvers, Mass.) containing protease inhibitors (Protease Inhibitor Cocktail set I, Calbiochem, Gibbstown, N.J.) for 1 hr and frozen at −80° C. BCA assay (Thermo/Pierce, Rockford, Ill.) quantified protein in samples. Ten ug protein boiled 3 minutes in Lammeli's buffer plus b-mercaptoethanol was loaded per lane in 10-20% tris-glycine gel (Invitrogen, Carlsbad, Calif.) and run at 125 mW for 1.5 hours. Proteins transferred to 0.2 um pore nitrocellulose membrane in transfer buffer (Invitrogen) 25 mW for 1 hour. Blots were blocked with 5% milk in PBS/0.1% Tween-20 (PBST) 1 hour and 0.2 ug/ml goat-anti-IL-1β (R&D cat# AF-401-NA) or 0.5 ug/mlrat-anti-caspase-1 (Genentech, clone 4B4.2) added in 1% milk/PBST 1 hour. Blots were washed 4×5 minutes in PBST. Anti-goat-HRP (1:5000 R&D) or anti-rat-HRP (Thermo/Pierce) was added for 45 minutes and washed 5×5 minutes in PBST. Blots were developed with ECL Plus and hyperfilm (GE Healthcare, Buckinghamshire, UK).

### Immunohistochemistry

Ten week old Balbc mice were injected subretinally with 1 μl of 1×10⁹ AAV2/5-IL1β or AAV2/5 empty control virus (Genedetect). After 7 weeks infection, whole eye was removed and fixed in 10% neutral buffered formalin overnight at room temperature. Sections were processed and embedded in paraffin then stained with anti-CD45 and visualized with DAB.

### Fluorescence Angiography (FA)

Animals were anesthetized by intraperitoneal injection of ketamine/xylazine (80 mg/kg:15 mg/kg) and eyes dilated with 1% tropicamide (Bausch and Lomb, Rochester, N.Y.). Eyes were kept moist with artificial tears. Mice were injected intraperitoneally with 100 μl 10% AK-Fluor injectable fluorescein (Akorn, Buffalo Grove, Ill.). Images were acquired with a Heidelberg Spectralis HRA/OCT camera (Heidelberg Engineering, Vista, Calif.).

### Optical Coherence Tomography (OCT)

Animals were anesthetized by intraperitoneal injection of ketamine/xylazine (80 mg/kg:15 mg/kg) and eyes dilated with 1% tropicamide (Bausch and Lomb, Rochester, N.Y.). Eyes were kept moist with artificial tears. Images were acquired with a Heidelberg Spectralis HRA/OCT camera (Heidelberg Engineering, Vista, Calif.). Measurements are an average thickness of 19 sections over a 15.2 mm² area of retina. Thickness includes retinal and choroid.

### Electroretinogram (ERG) Recordings

Mice were dark adapted for 24 hours before ERG to equilibrate retinal responses. Once dark adapted, all subsequent procedures will be performed in the dark with only a red light for illumination. Animals were anesthetized with intraperitoneal injection of Ketamine and Xylazine (75-80 mg/kg:7.5-15 mg/kg). Mouse body temperature was maintained at 37° C. using a homeothermic heating plate connected to its control unit. Pupils were dilated with 1% atropine and the corneal surface was anesthetized with a drop of 0.5% proparacaine HCl. ERGs from both eyes were recorded simultaneously using an Espion E2 (Diagnosys LLC, Lowell, Mass.) visual electrophysiology system. Mice were placed on a platform and a reference electrode was inserted subcutaneously in the forehead and a ground electrode was inserted at the base of the tail. Gonak hypermellose solution was placed on the cornea to establish an electrical contact between the cornea and the platinum electrode and protected eyes from drying during the experiment. A mouse was placed in the ColorDome full field desktop Ganzfeld stimulator and were stimulated with white light: 3 flash intensities ranging 1×10⁻⁵-5 cd/m², allowing 2 minutes between flashes in order to reestablish baseline response. Signals were band pass-filtered at 0.15-1000 Hz and sampled at 2 kHz.

Methods for phage panning IL-1β

Several phage display synthetic antibody libraries were panned against immobilized human IL-1β. Enrichment of antibody displaying phage pools specific for IL-1β was determined at round three and subsequent rounds by measuring the ratio of recovered pools of phage clones specific for IL-1β over those specific for binding bovine serum albumin. Construction of the synthetic naïve antibody phage display libraries is described elsewhere (Sidhu et al., 2004). After several rounds of panning, phage clones displaying antibody variable heavy and light chain domains specific for IL-1β were identified. The DNA sequences of the variable heavy and chain were determined and reformatted into human IgG1 expression vectors to allow transient antibody expression in mammalian cells. Antibody from the cell culture growth media was purified using Protein A for subsequent testing in soluble protein binding affinity determination assays, receptor-ligand inhibition assays and functional cell based assays.

### Competitive Inhibition of Human IL-1β Binding to Human IL-1R1 or IL-1RII

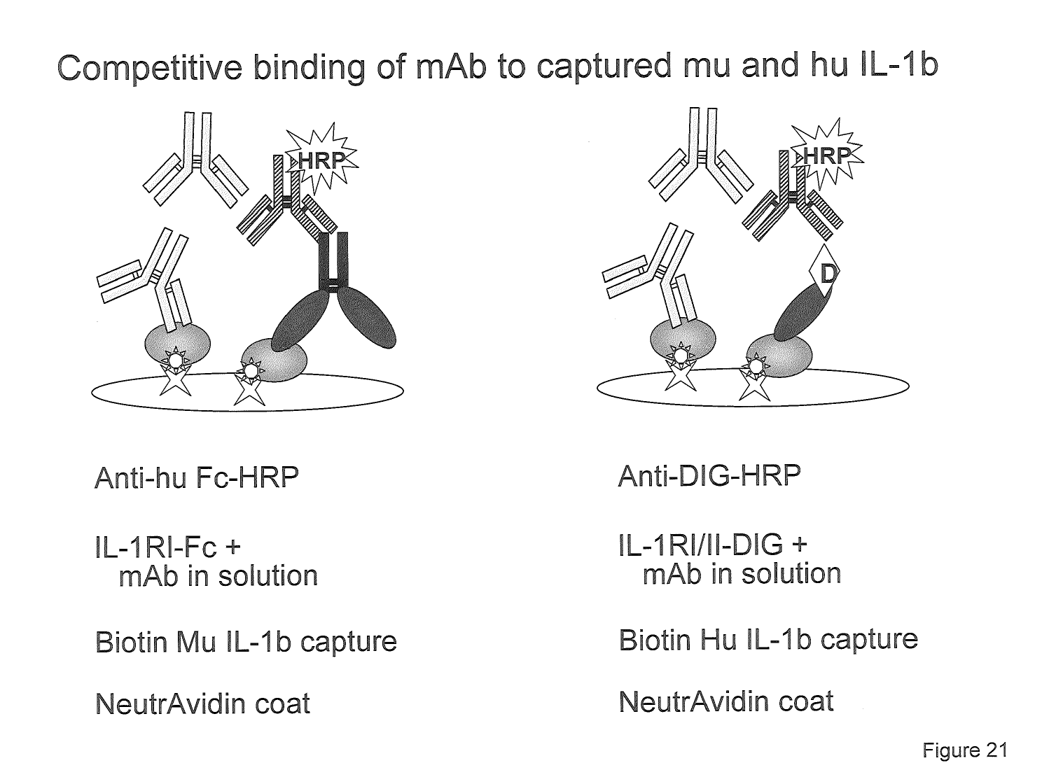

NeutrAvidin (Pierce, Rockford, Ill.) was diluted to 2 μg/mL in phosphate buffered saline (PBS) and coated on ELISA plates (384-well high-bind plates, Nunc, Neptune, N.J.) during an overnight incubation at 4° C. After washing three times with wash buffer (PBS/0.05% Tween-20), the plates were blocked with PBS/0.5% bovine serum albumin (BSA) for 1 to 2 hours. This and all subsequent incubations were performed at room temperature on an orbital shaker. Human IL-1p (R&D Systems, Minneapolis, Minn.) biotinylated using maleimide-PEG-biotin (Pierce) according to the manufacturer's directions was diluted to 400 ng/ml in assay buffer (PBS/0.5% BSA/0.05% Tween-20). The blocked NeutrAvidin plates were washed, and biotinylated human IL-1β was captured onto the plates during a 1-2 hr incubation. Human IL-1RI and IL-1RII (R&D Systems) were labeled with digoxigenin (DIG) using 3-amino-3-deoxydigoxigenin hemisuccinamide succinimidyl ester (Invitrogen, Eugene, Oreg.) according to the manufacturer's directions. The ability of antibodies to block the binding of IL-1RI and IL-1RII to IL-1β was evaluated by diluting the antibodies over a broad range and mixing them with equal volumes of DIG-labeled human IL-1RI or IL-1RII (final concentrations of 1 μg/ml or 60 ng/ml, respectively). The mixtures were added to washed plates and allowed to incubate for 1-2 hr. Plate-bound IL-1RI or IL-1RII was then detected using a horseradish peroxidase (HRP)-conjugated monoclonal anti-DIG antibody (Jackson ImmunoResearch, West Grove, Pa.). After a 1 hr incubation and an additional wash step, tetramethyl benzidine (TMB, Kirkegaard & Perry Laboratories, Inc., Gaithersburg, Md.) was added, and color was allowed to develop for approximately 10 min. The reaction was stopped by the addition of 1 M phosphoric acid. The optical density was read using a microplate reader (450 nm, 650 nm reference), and antibody concentrations yielding half maximal inhibition of binding were determined using four-parameter fits of the curves (Kaleidagraph, Synergy Software, Reading, Pa.). See FIG. 21.

### Competitive Inhibition of Mouse IL-1β Binding to Mouse IL-1RI or IL-1RII

The ability of antibodies to block binding of mouse IL-1β to mouse IL-1RI and IL-1RII was evaluated using a similar method. Mouse IL-1β (R&D Systems) was biotinylated using sulfo-NHS-LC-biotin (Pierce) according to the manufacturer's directions and captured onto NeutrAvidin plates at a concentration of 400 ng/ml. Antibodies were diluted over a broad range, mixed with an equal volumes of mouse IL-1RI- or IL-1RII-human IgG1 Fc fusion proteins (R&D Systems; final concentrations of 1 μg/ml or 60 ng/ml, respectively), and incubated for 1-2 hr on the prepared plates. Bound receptor was detected using an HRP-conjugated goat polyclonal anti-human IgG Fc antibody (Jackson ImmunoResearch). Color development and data analysis were performed as described above. See FIG. 21.

### Competitive Inhibition of Human IL-18 Binding to Human IL-18Ra

The overall assay method was essentially the same as described for evaluating inhibition of human IL-1β/IL-1R binding. ELISA plates were coated with NeutrAvidin (Pierce), and human IL-18 (R&D Systems) biotinylated using sulfo-NHS-LC-biotin (Pierce) was diluted to 400 ng/ml and captured onto the plates. Human IL-18Ra-human IgG1 Fc (R&D Systems) was labeled with digoxigenin (DIG) using 3-amino-3-deoxydigoxigenin hemisuccinamide succinimidyl ester (Invitrogen, Eugene, Oreg.). Diluted antibodies were mixed with equal volumes of DIG-IL-18Ra-Fc (final concentration of 1 μg/ml). Bound receptor was detected using an anti-DIG antibody (Jackson ImmunoResearch). Color development and data analysis were performed as described above.

### Example 1

### Combined IL-1β and IL-18 Blockade in Inflammatory Bowel Disease

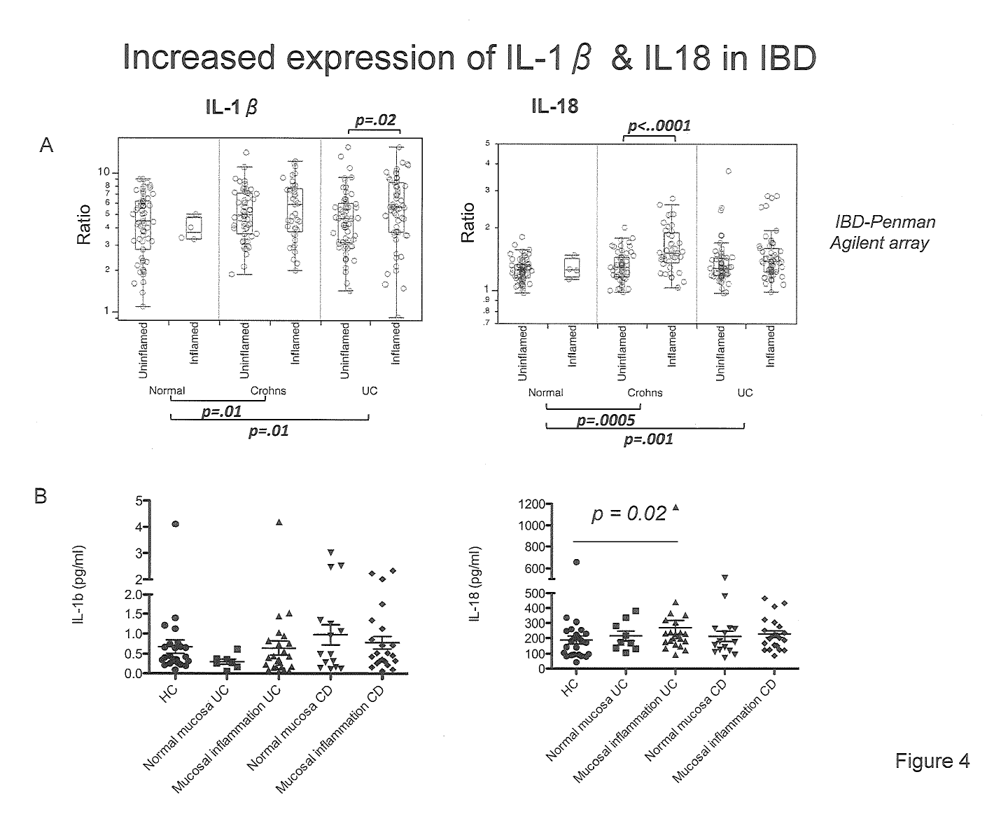

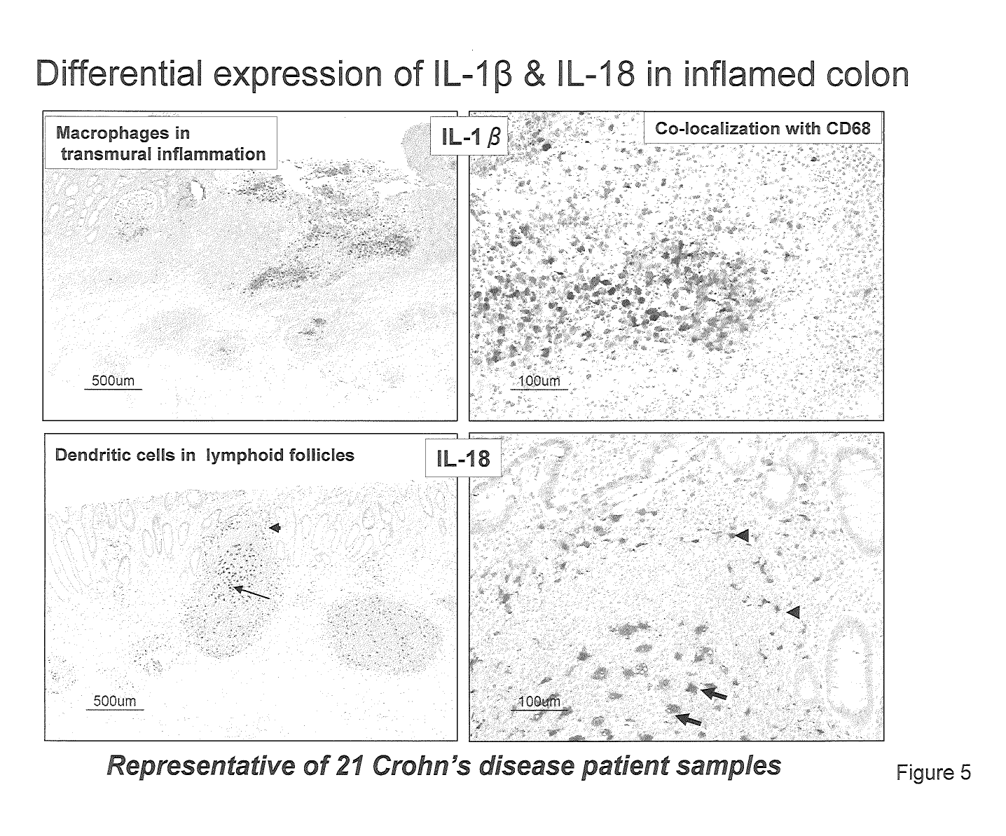

In clinical studies, the present inventors have found a significant increase in IL-1β and IL-18-expressing cells in Crohn's disease, as well as significantly increased serum IL-18 levels in Crohn's disease (See FIG. 4). In preclinical mouse models of IBD, an increase in IL-1β and IL-18 secretion from the colon in an ex-vivo colon culture was found (see FIG. 5). For IL-1β, positive cells are at sites of active inflammation with few or no positive cells in areas without evidence of active inflammation (FIG. 5, upper photos). For IL-18, positive cells are morphologically compatible with follicular dendritic cells (arrows) and myeloid dendritic cells in the marginal zone (arrowheads) of the lymphoid follicle (FIG. 5, lower photos). IL-18 positive cells are also colon epithelial cells. These results are representative of 21 Crohn's disease patient samples evaluated.

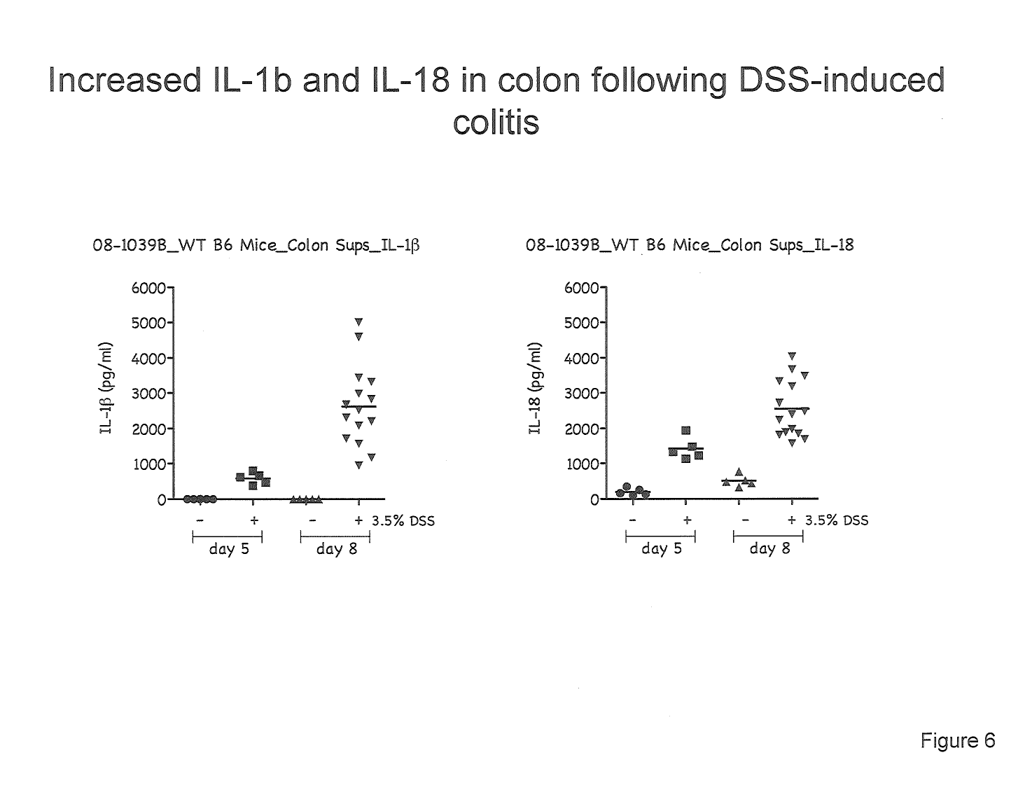

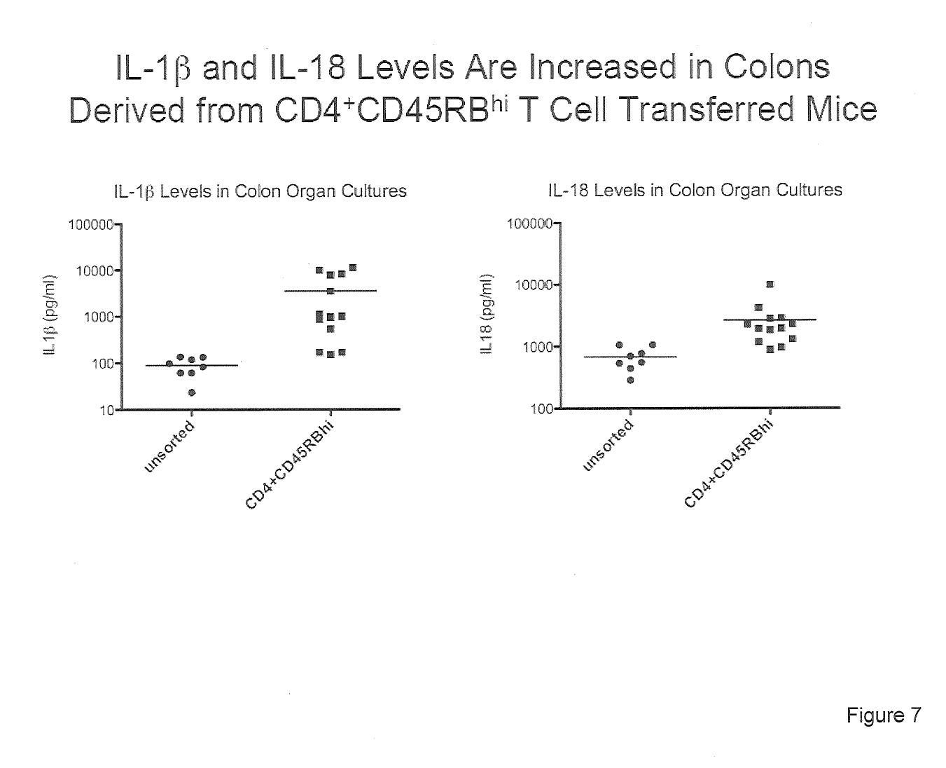

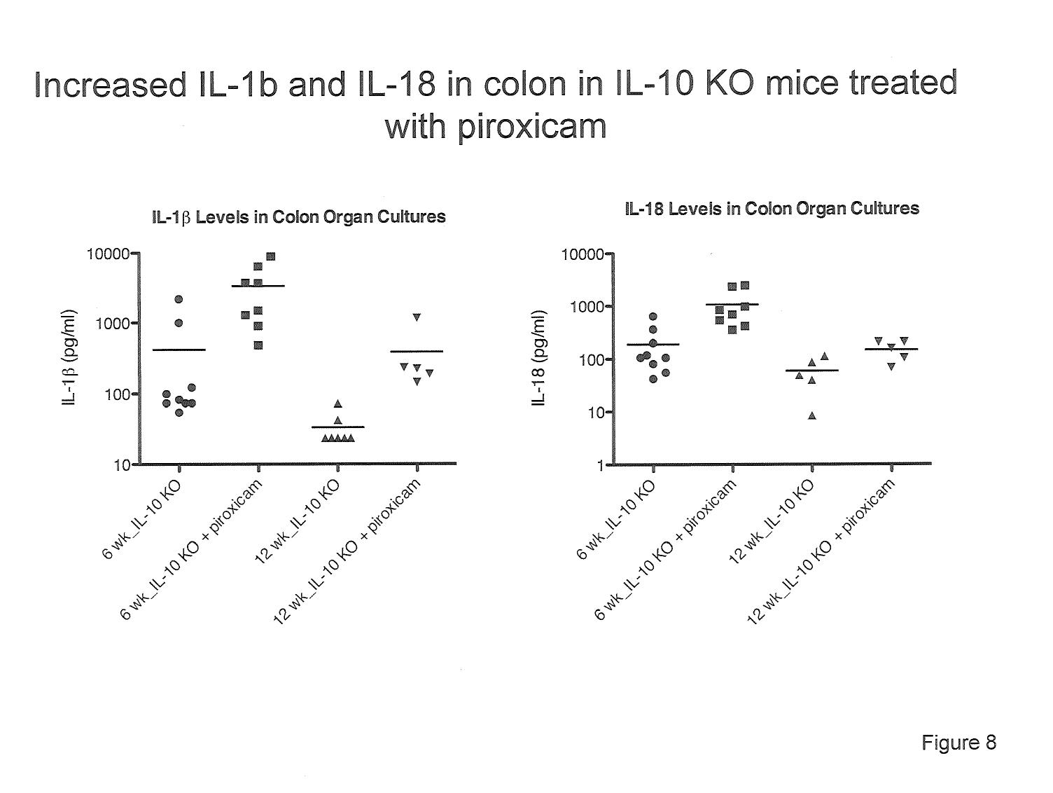

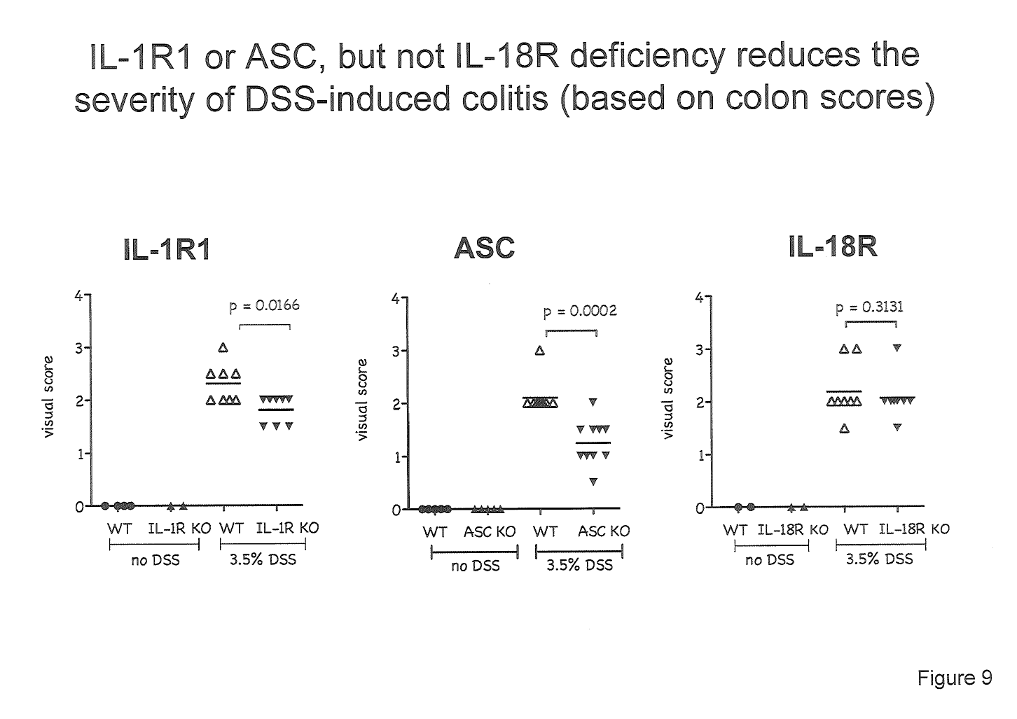

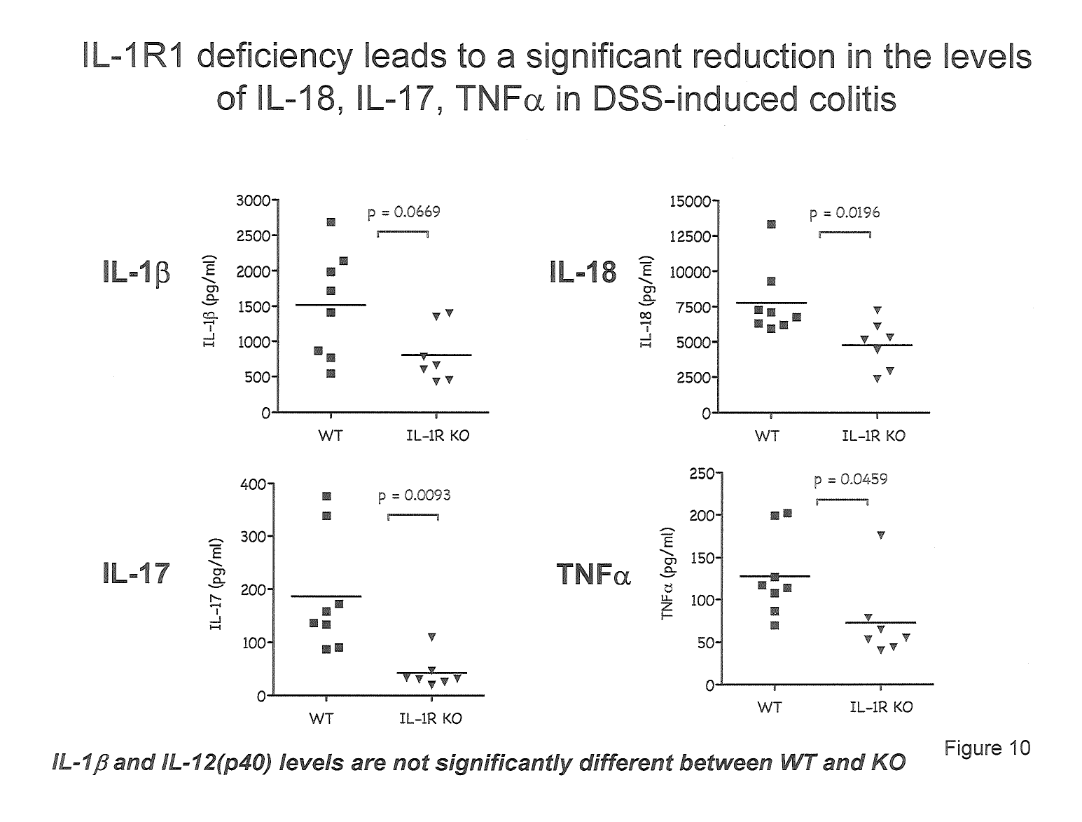

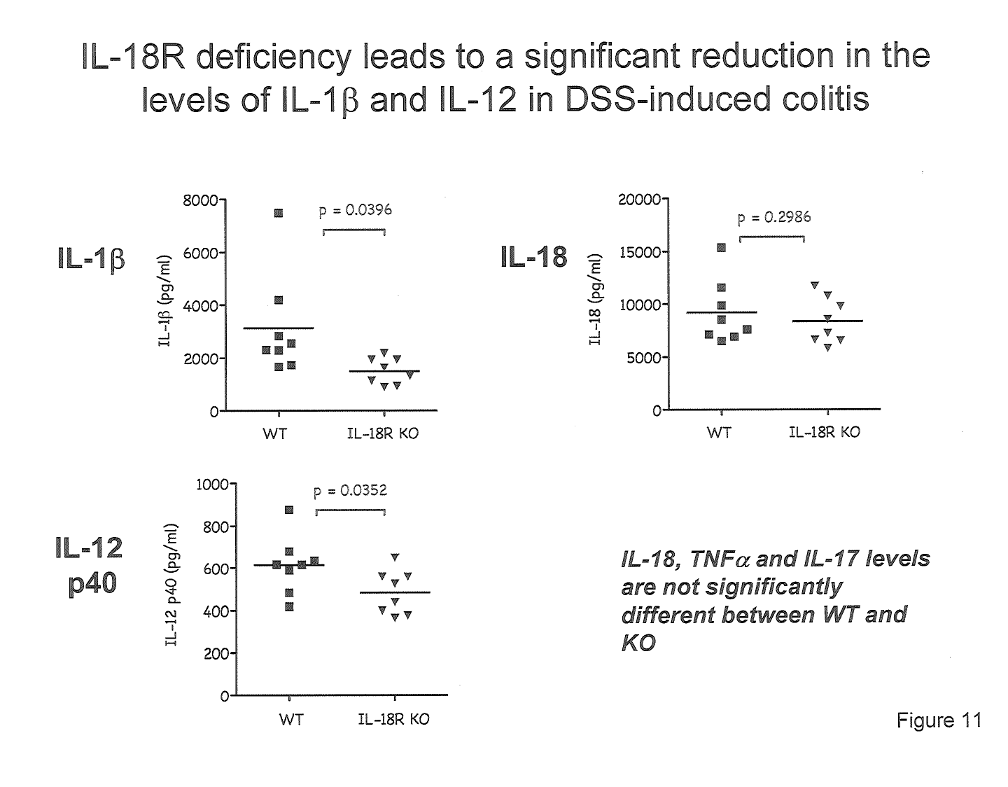

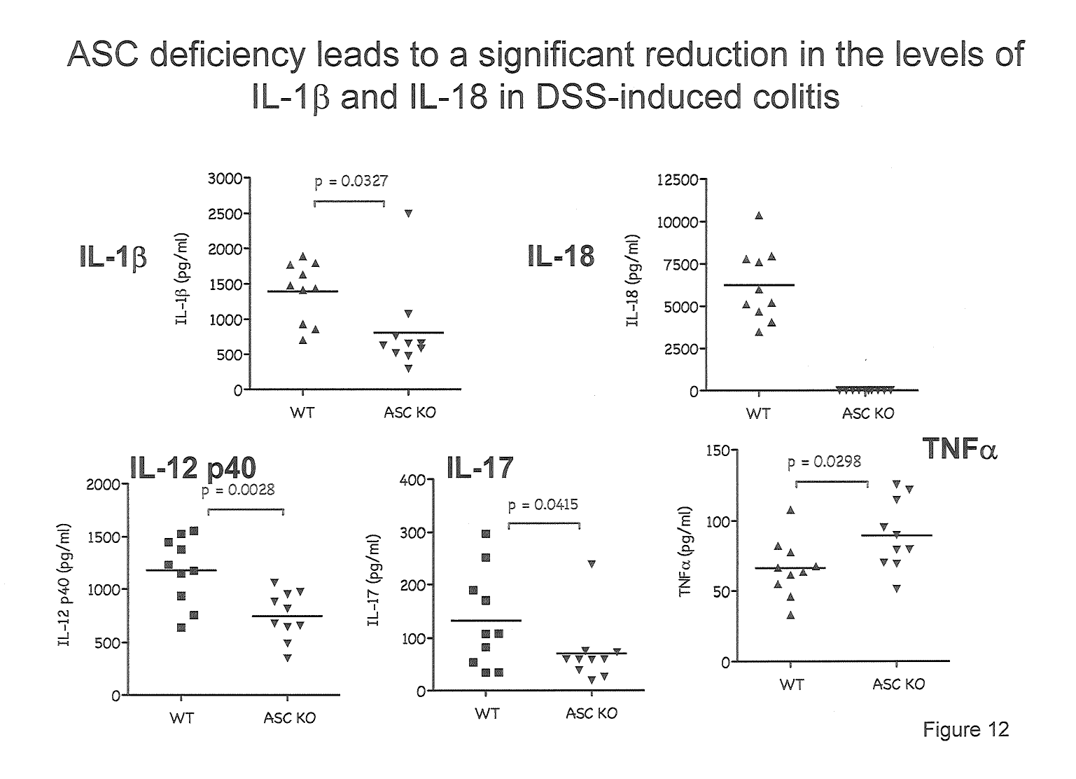

Mouse models studied included DSS-induced colitis (in WT B6 female mice), T-cell adoptive transfer and piroxicam-IL-10 KO (see FIGS. 6, 7 and 8). The present inventors have demonstrated that blockade of IL-1β, IL-18 or both (in the case of ASC KO studies) reduces inflammatory response (IL-1β, IL-18, TNFα, IL-17, IL-6) and colon scores in the DSS model of colitis (see FIGS. 9-13).

### Example 2

### Combined IL-1β and IL-18 blockade in Age-Related Macular Degeneration

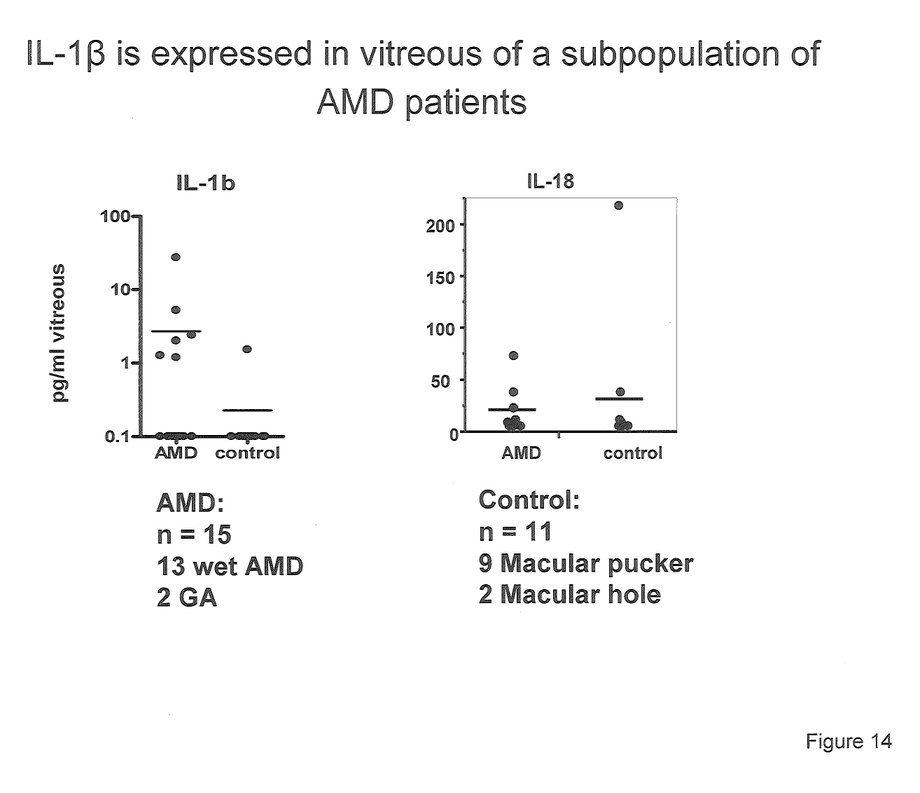

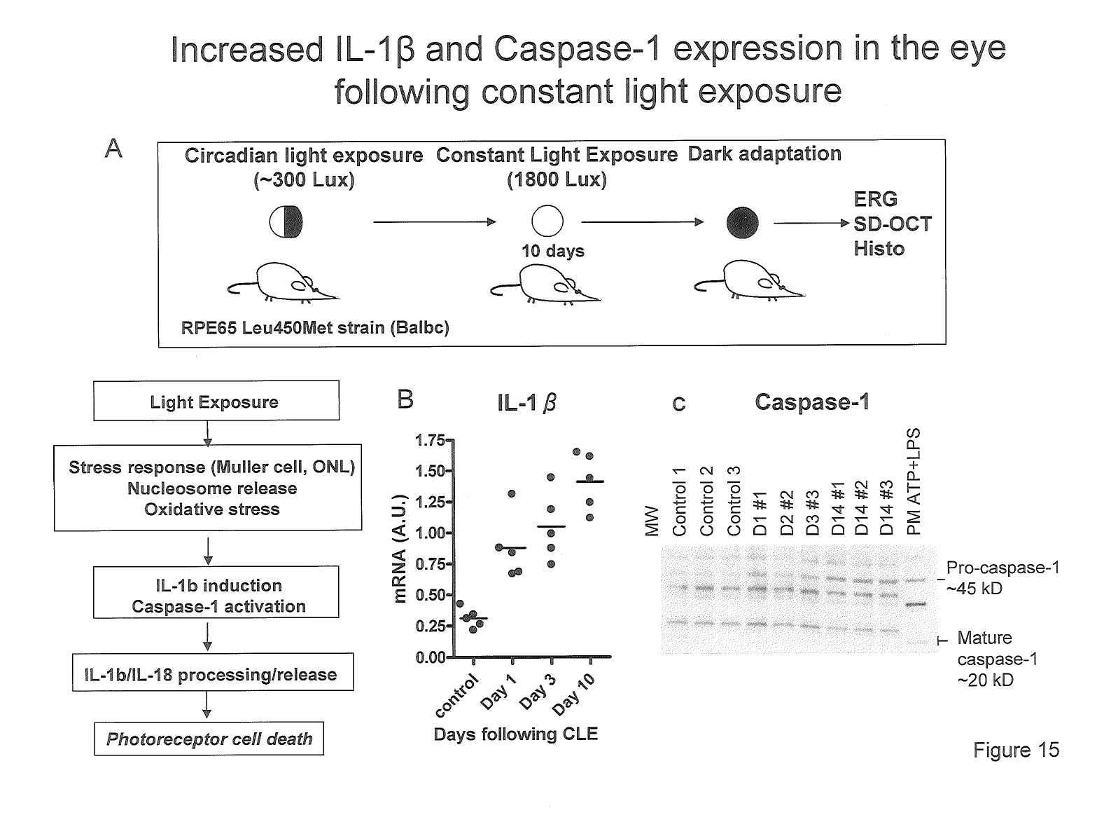

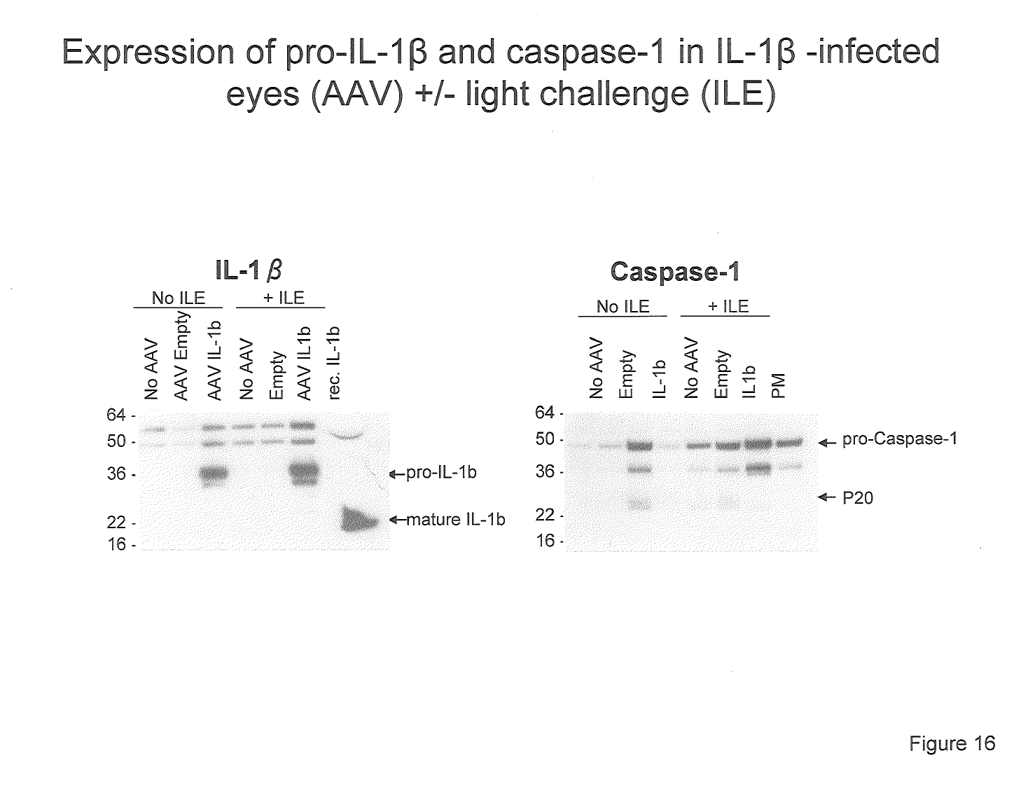

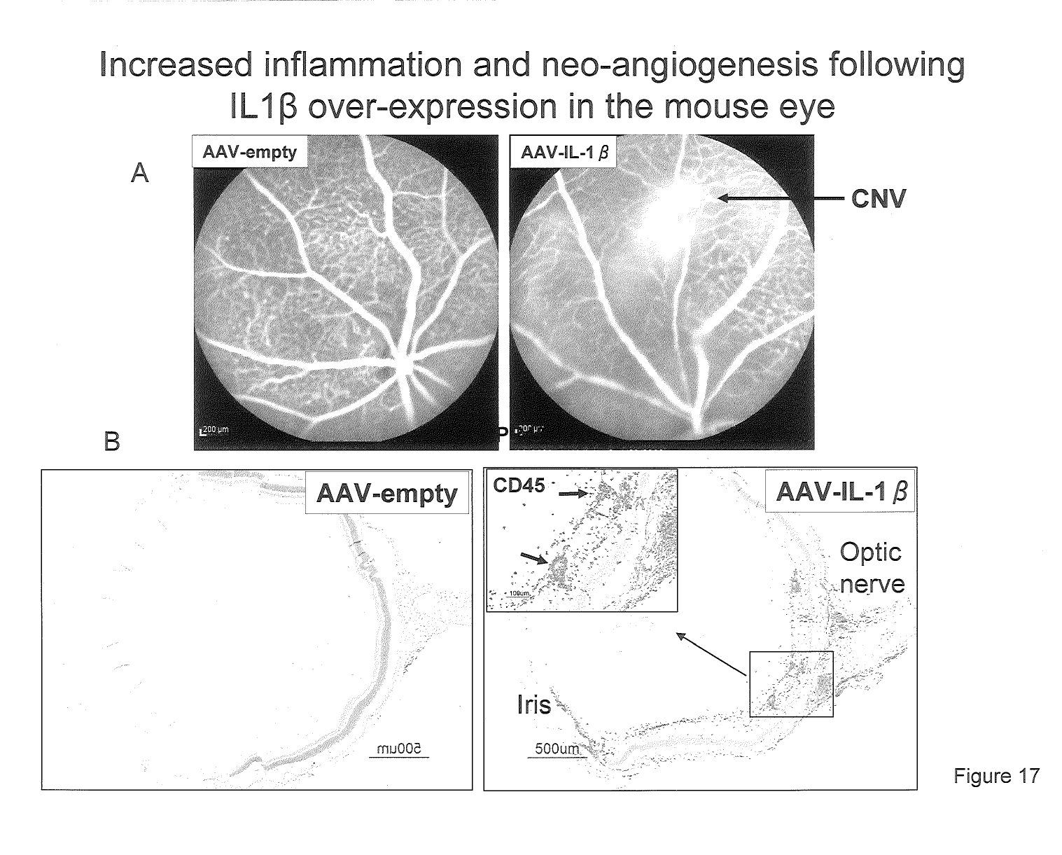

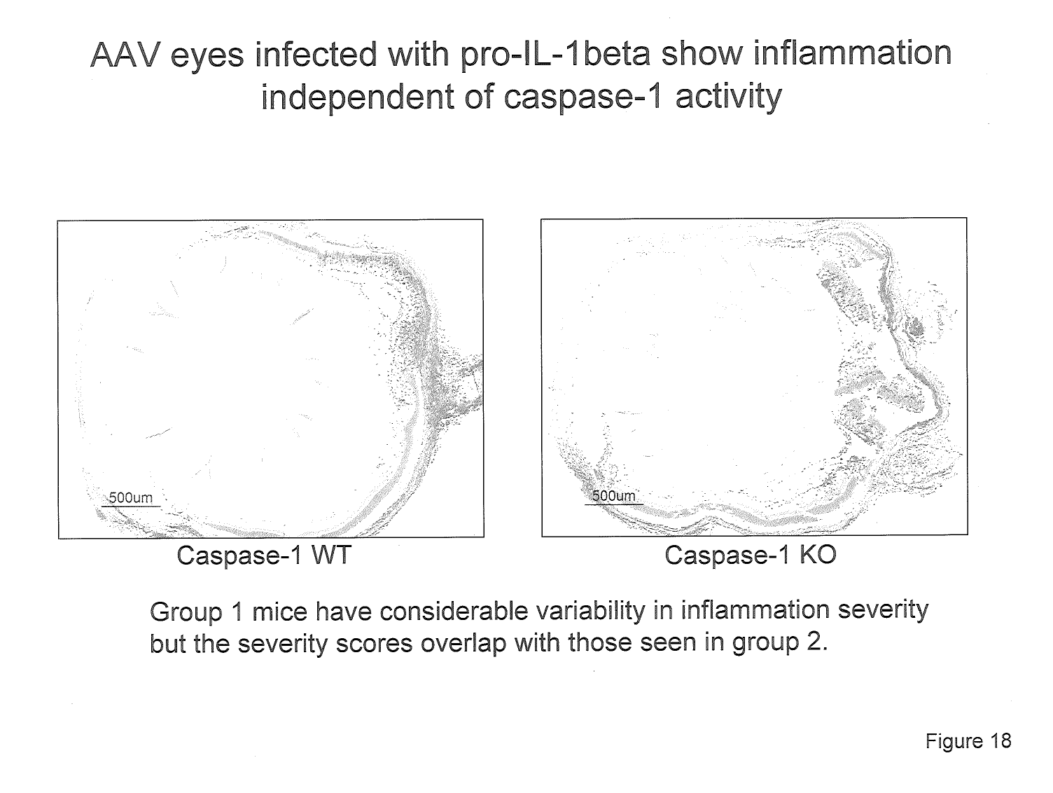

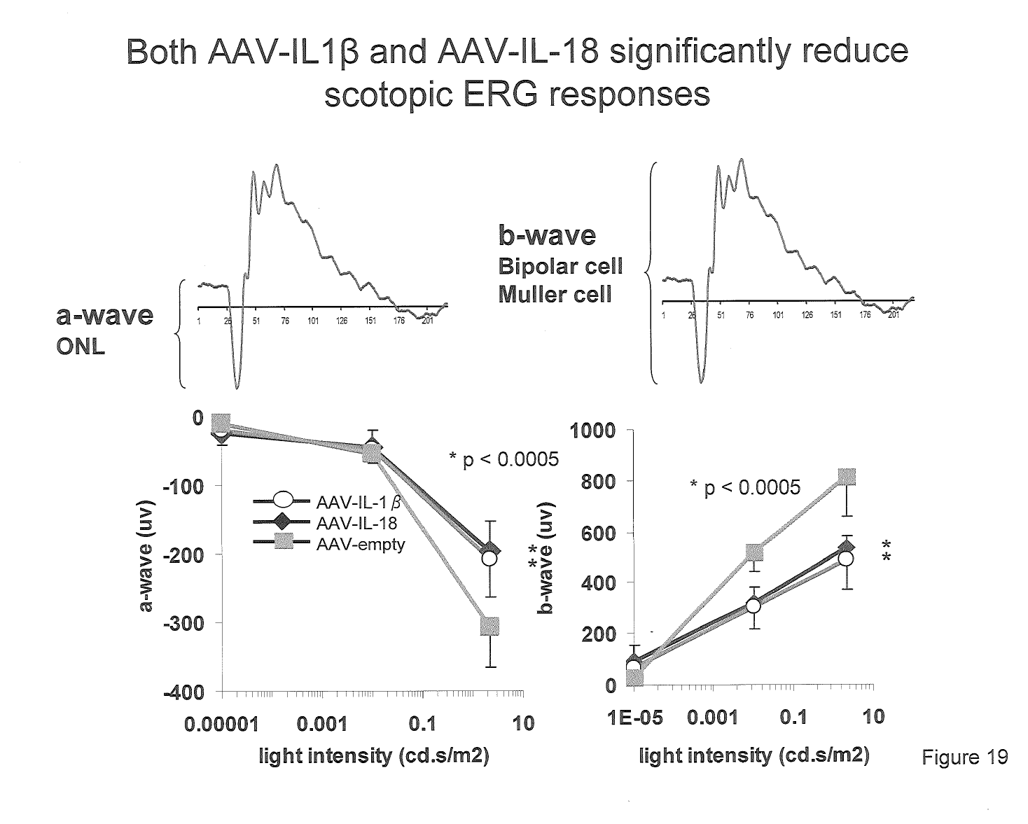

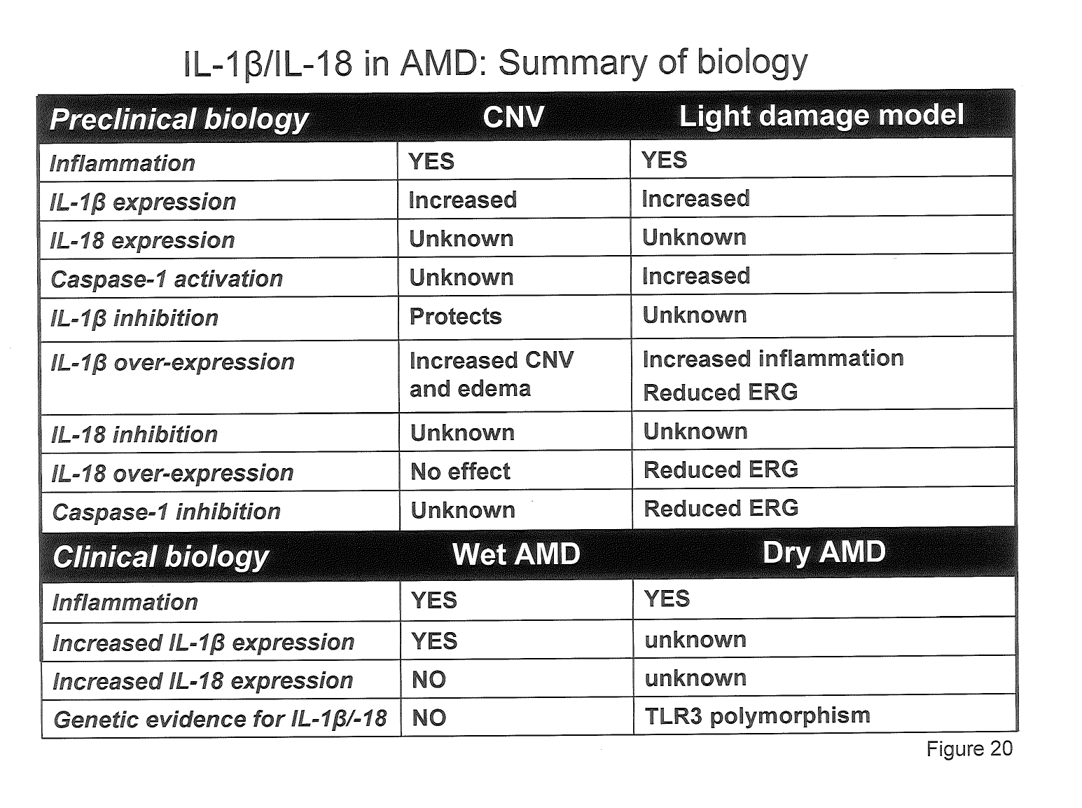

Previous studies reported that IL-1β is increased in vitreous fluid of patients with diabetic retinopathy and uveitis. However, no studies have reported on the presence of IL-1β and IL-18 in wet or dry AMD. The present studies show that IL-1β levels are increased in vitreous of a subpopulation of AMD patients (see FIG. 14). In preclinical mouse studies, the present inventors show that over-expression of IL-1β in the mouse eye induces retinal inflammation, while IL-18 over-expression does not (see FIGS. 15-18). Further, the present inventors show that both, IL-1β and IL-18, affect retinal function as measured by ERG recordings (see FIG. 19). Based on these studies, the present inventors conclude that single and combined IL-1β and IL-18 blockade is expected to improve photoreceptor function and CNV/edema (see FIG. 20).

### Example 3

### Combined IL-10 and IL-18 blockade in Type 2 Diabetes Mellitus

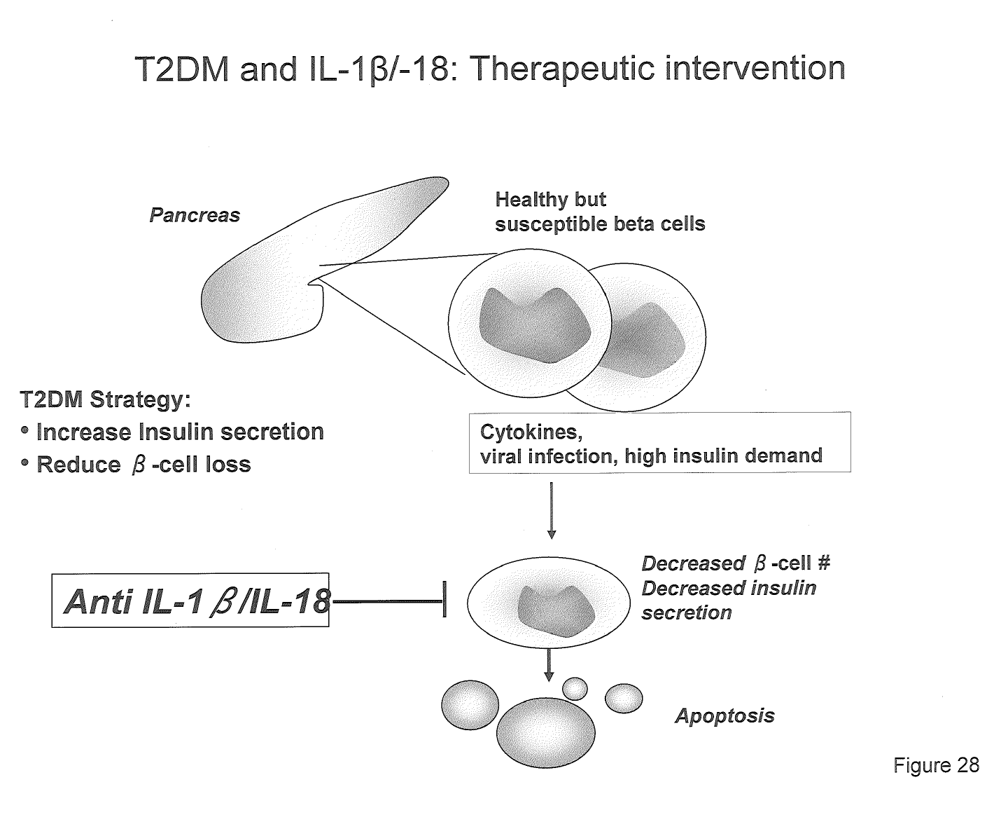

The present inventors hypothesize that targeting IL-1β may preserve β-cell functions in patients with type 2 diabetes. IL-1β reportedly decreases insulin secretion by pancreatic 3 cells in vitro and alters various β-cell functions. Further, treatment with IL-1Ra reportedly may prevent or ameliorate animal models of diabetes, and IL-1Ra is reportedly decreased in β cells obtained from patients with type 2 diabetes. See FIG. 28.

Gene polymorphisms in the IL-1β/IL-18 pathway are reportedly associated with central obesity and metabolic syndrome (Carter et al., 2008). Further, IL-1β reportedly decreases insulin secretion by pancreatic β cells in vitro (Lewis and Dinarello, 2006), and Anakinra (IL-1Ra) reportedly improves glycemia and beta cell secretory function in patients (Larsen et al. 2007). Additionally, increased serum levels of IL-1 and IL-18, reportedly decreased the ratio to IL-1Ra and IL-18BP in T2DM patients, and IL-1Ra and IL-18BP protect against STZ or high-fat induced hyperglycemia in preclinical models (Sandberg et al., 1994).

Further, Larsen et al, carried out a double-blind clinical trial in patients with type 2 diabetes by administering anakinra once daily for 13 weeks (Larsen et al., 2007). This treatment improved glycemia and β-cell insulin secretory capacity as well as reduced markers of systemic inflammation. However, there remains a need to determine the possible beneficial effects of anti-IL-1 therapies possessing a more prolonged half-life and administered over a longer period of time on restoration of β-cell mass and function in patients with type 2 diabetes.

### Example 4

### Anti-IL-1b and/or Anti-IL18 in the Piroxicam IL-10KO IBD Model

IL-10−/− mice develop spontaneous colitis. However, the incidence and severity are inconsistent, which make it harder to be used as model for IBD to test our therapeutics. By feeding the IL-10−/− mice with piroxicam, it is likely that piroxicam will exacerbate the chronic intestinal inflammation in these mice and may synchronize the onset of the colitis as indicated by Berg et al. (2002). Thus, this is a chronic inflammation model of IBD in contrast to the acute DSS model of IBD.

6-wk old female IL-10KO (Genentech) mice were divided into the following treatment groups:

| Group | Agent | Dose | Frequency | Route | N |
|---|---|---|---|---|---|
| 1 | anti-ragweed | 1 mg/mouse | 3 times a week | i.p. | 11 |
| | anti-gp120 | 1 mg/mouse | | | |
| 2 | TNFRII-Fc | 300 ug/mouse | 3 times a week | i.p. | 12 |
| 3 | anti-IL-1b | 1 mg/mouse | 3 times a week | i.p. | 12 |
| 4 | anti-IL-18 | 1 mg/mouse | 3 times a week | i.p. | 12 |
| 5 | anti-IL-1b | 1 mg/mouse | 3 times a week | i.p. | 12 |
| | anti-IL-18 | 1 mg/mouse | 3 times a week | i.p. | 12 |

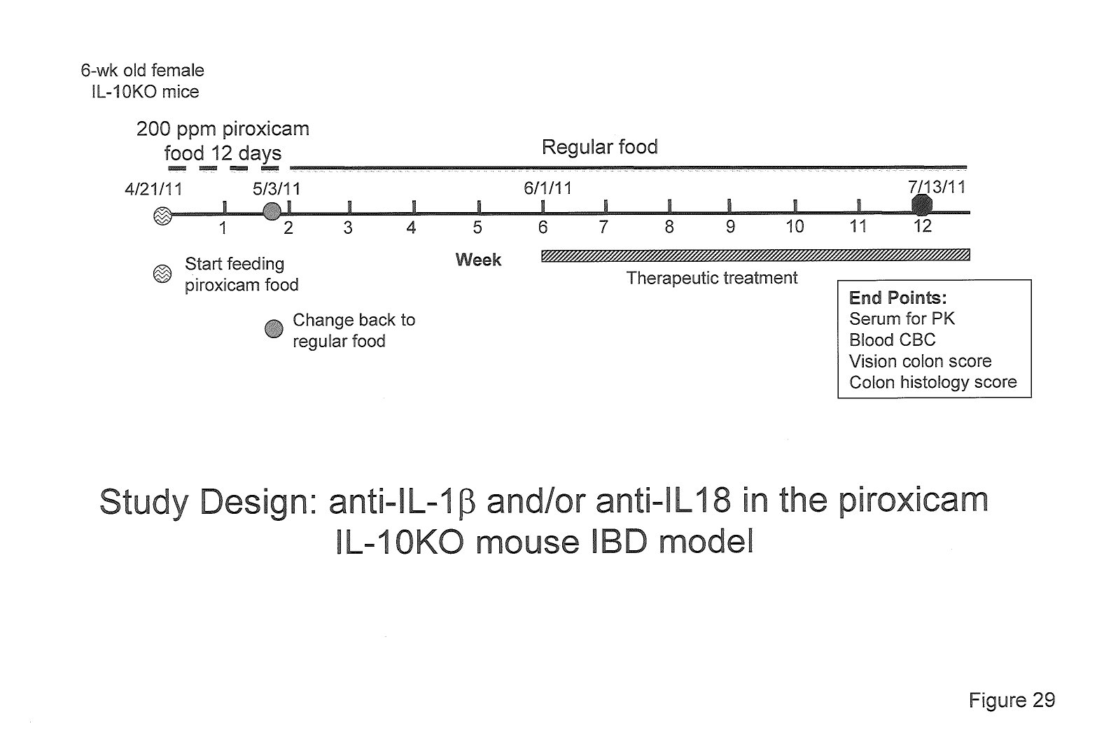

Piroxicam powder was mixed with powdered rodent diet at the concentration of 200 ppm using geometric dilution. Briefly, an equivalent amount of mouse diet was added to the piroxicam and then mixed thoroughly. Successive equivalent amounts of the mouse diet were added, mixing well after each dilution, until the entire quantity of the mouse diet was incorporated. After overnight fasting, mice were fed on the piroxicam containing diet for 11 days, and regular diet was put back on Day 12. All treatments were injected at the amount indicated above in 400 μl PBS 3 times a week i.p. for 6 weeks. Animals were weighed daily and sacrificed at the end of the study for analysis. Before the start of prioxicam treatment, 100 μl of blood was collected through tail vein by tail nick for FACS and serum. Then, 100 μl of blood will be collected at week 5 after the start of the experiment. In these studies, treatment effects on visual colon score, colon histology and serum PK were analyzed. See FIG. 29.

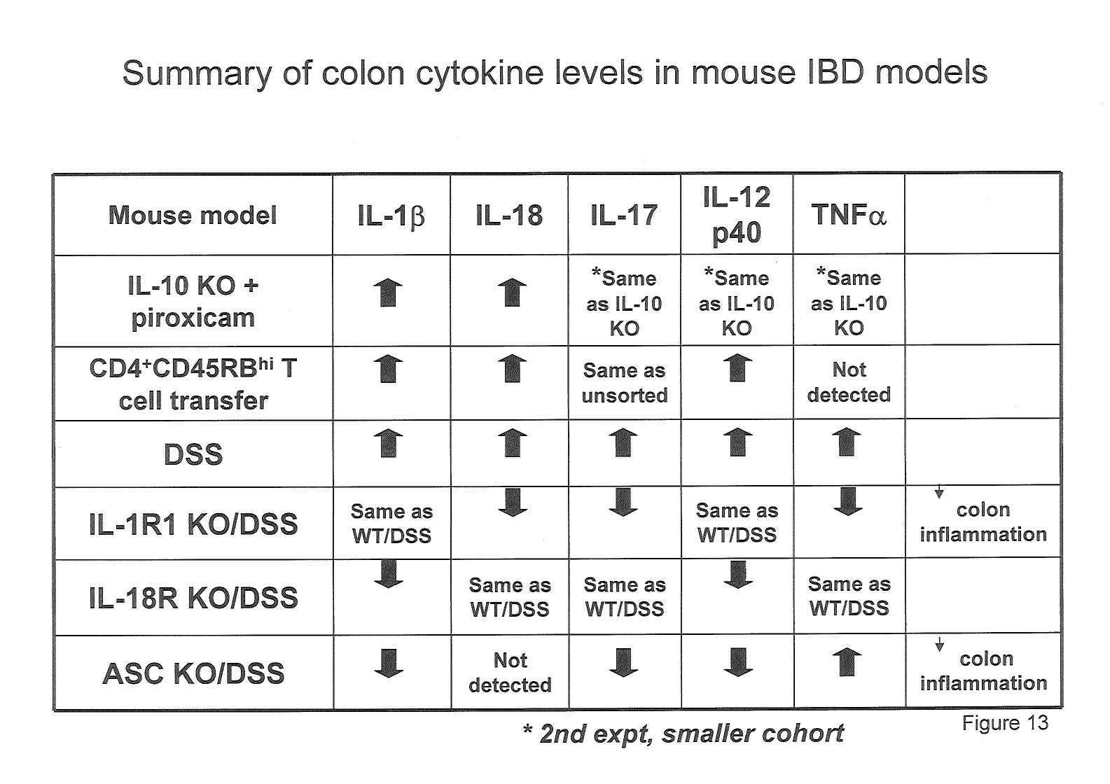

Levels of various cytokines were measured in IL-10 KO mice with and without piroxicam treatment. See FIG. 13. As noted, there is an elevation of IL-18 and IL-18 in the piroxicam treatment group (relative to the WT animals) while TNFα, IL-12 and IL-17 were comparable between the groups.

For histopathologic analyses, tissues were fixed in 10% formalin and subsequently embedded in paraffin for sectioning and haematoxylin and eosin staining. Histopathology scores were assessed in the proximal, medial and distal colon as well as the rectum and scored on a scale of 1 to 3. The scores for the individual colon segments were summed to yield the total score per animal. The same individual scored all histologic features and had no knowledge of the experimental groups.

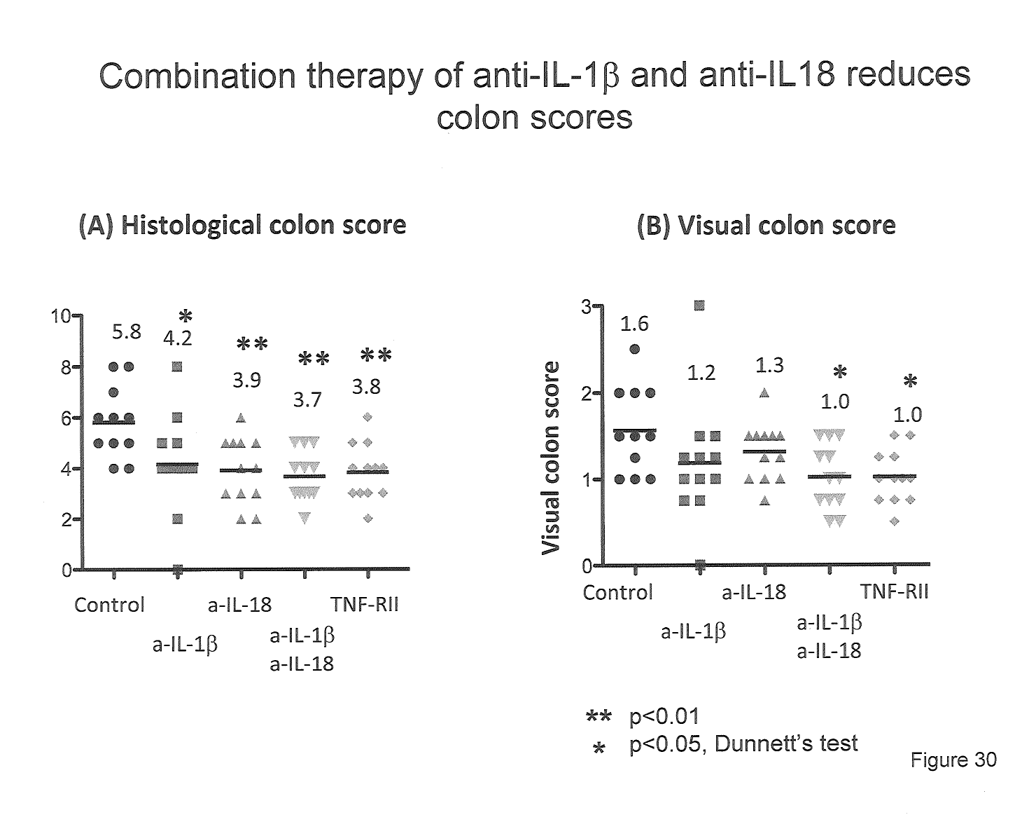

Results for the visual colon scores as well as the histology scores are shown in FIG. 30. Serum levels were elevated for IL-1β and IL-18. Treatment with a combination of anti-IL-1β and anti-IL-18 antibodies resulted in statistically significant reduction of injury to the colon. The combination treatment was as effective as TNFRII-Fc treatment. These results demonstrate that the combined blockade of IL-1β and IL-18 can be an effective therapy for IBD. Combined blockade of IL-1β and IL-18 may also provide a safer treatment than TNF-alpha blockade.

### REFERENCES

Arend, W. P., G. Palmer, and C. Gabay. 2008. IL-1, IL-18, and IL-33 families of cytokines. Immunol Rev. 223:20-38.
Baldassano, R. N., J. P. Bradfield, D. S. Monos, C. E. Kim, J. T. Glessner, T. Casalunovo, E. C. Frackelton, F. G. Otieno, S. Kanterakis, J. L. Shaner, R. M. Smith, A. W. Eckert, L. J. Robinson, C. C. Onyiah, D. J. Abrams, R. M. Chiavacci, R. Skraban, M. Devoto, S. F. Grant, and H. Hakonarson. 2007. Association of the T300A non-synonymous variant of the ATG16L1 gene with susceptibility to paediatric Crohn's disease. Gut. 56:1171-3.
Cadwell, K., J. Y. Liu, S. L. Brown, H. Miyoshi, J. Loh, J. K. Lennerz, C. Kishi, W. Kc, J. A. Carrero, S. Hunt, C. D. Stone, E. M. Brunt, R. J. Xavier, B. P. Sleckman, E. L1, N. Mizushima, T. S. Stappenbeck, and H. W. t. Virgin. 2008. A key role for autophagy and the autophagy gene Atg16l1 in mouse and human intestinal Paneth cells. Nature. 456:259-63.
Carter, K. W., J. Hung, B. L. Powell, S. Wiltshire, B. T. Foo, Y. C. Leow, B. M. McQuillan, M. Jennens, P. A. McCaskie, P. L. Thompson, J. P. Beilby, and L. J. Palmer. 2008. Association of Interleukin-1 gene polymorphisms with central obesity and metabolic syndrome in a coronary heart disease population. Hum Genet. 124:199-206.
Cassel, S. L., S. Joly, and F. S. Sutterwala. 2009. The NLRP3 inflammasome: A sensor of immune danger signals. Semin Immunol.
Ferrero-Miliani, L., O. H. Nielsen, P. S. Andersen, and S. E. Girardin. 2007. Chronic inflammation: importance of NOD2 and NALP3 in interleukin-1beta generation. Clin Exp Immunol. 147:227-35.
Kowluru, R. A., and S. Odenbach. 2004. Role of interleukin-1beta in the pathogenesis of diabetic retinopathy. Br J. Ophthalmol. 88:1343-7.
Kuballa, P., A. Huett, J. D. Rioux, M. J. Daly, and R. J. Xavier. 2008. Impaired autophagy of an intracellular pathogen induced by a Crohn's disease associated ATG16L1 variant. PLoS One. 3:e3391.
Larsen, C. M., M. Faulenbach, A. Vaag, A. Volund, J. A. Ehses, B. Seifert, T. Mandrup-Poulsen, and M. Y. Donath. 2007. Interleukin-1-receptor antagonist in type 2 diabetes mellitus. N Engl J. Med. 356:1517-26.
Lewis, E. C., and C. A. Dinarello. 2006. Responses of IL-18- and IL-18 receptor-deficient pancreatic islets with convergence of positive and negative signals for the IL-18 receptor. Proc Natl Acad Sci USA. 103:16852-7.
Ludwiczek, O., A. Kaser, D. Novick, C. A. Dinarello, M. Rubinstein, and H. Tilg. 2005. Elevated systemic levels of free interleukin-18 (IL-18) in patients with Crohn's disease. Eur Cytokine Netw. 16:27-33.
Ludwiczek, O., E. Vannier, I. Borggraefe, A. Kaser, B. Siegmund, C. A. Dinarello, and H. Tilg. 2004. Imbalance between interleukin-1 agonists and antagonists: relationship to severity of inflammatory bowel disease. Clin Exp Immunol. 138:323-9.
Monteleone, G., F. Trapasso, T. Parrello, L. Biancone, A. Stella, R. luliano, F. Luzza, A. Fusco, and F. Pallone. 1999. Bioactive IL-18 expression is up-regulated in Crohn's disease. J. Immunol. 163:143-7.
Perrier, S., F. Darakhshan, and E. Hajduch. 2006. IL-1 receptor antagonist in metabolic diseases: Dr Jekyll or Mr Hyde? FEBS Lett. 580:6289-94.
Saitoh, T., N. Fujita, M. H. Jang, S. Uematsu, B. G. Yang, T. Satoh, H. Omori, T. Noda, N. Yamamoto, M. Komatsu, K. Tanaka, T. Kawai, T. Tsujimura, O. Takeuchi, T. Yoshimori, and S. Akira. 2008. Loss of the autophagy protein Atg16L1 enhances endotoxin-induced IL-1beta production. Nature. 456:264-8.
Sandberg, J. O., A. Andersson, D. L. Eizirik, and S. Sandler. 1994. Interleukin-1 receptor antagonist prevents low dose streptozotocin induced diabetes in mice. Biochem Biophys Res Commun. 202:543-8.
Sidhu, S. S., B. L1, Y. Chen, F. A. Fellouse, C. Eigenbrot, and G. Fuh. 2004. Phage-displayed antibody libraries of synthetic heavy chain complementarity determining regions. J Mol. Biol. 338:299-310.
Ten Hove, T., A. Corbaz, H. Amitai, S. Aloni, I. Belzer, P. Graber, P. Drillenburg, S. J. van Deventer, Y. Chvatchko, and A. A. Te Velde. 2001. Blockade of endogenous IL-18 ameliorates TNBS-induced colitis by decreasing local TNF-alpha production in mice. Gastroenterology. 121:1372-9.
Villani, A. C., M. Lemire, G. Fortin, E. Louis, M. S. Silverberg, C. Collette, N. Baba, C. Libioulle, J. Belaiche, A. Bitton, D. Gaudet, A. Cohen, D. Langelier, P. R. Fortin, J. E. Wither, M. Sarfati, P. Rutgeerts, J. D. Rioux, S. Vermeire, T. J. Hudson, and D. Franchimont. 2009. Common variants in the NLRP3 region contribute to Crohn's disease susceptibility. Nat. Genet. 41:71-6.

What is claimed is:

1. A method of treating a disease in a patient, the method comprising administering to said patient an effective amount of:
a. An IL-1β/IL-18 bispecific antibody; or
b. An antibody that binds IL-1β and IL-18 activity; or
c. An antibody that binds IL-1β and an antibody that binds IL-18;
wherein said antibody or antibodies of parts a, b or c is/are capable of neutralizing or blocking IL-1β and IL-18 activity in cells or tissue.

2. The method of claim 1, wherein the antibody/antibodies is/are humanized.

3. The method of claim 1, wherein the antibody of part (b) is a dual action antibody.

4. The method of claim 1, wherein at least one antibody of part (c) is monoclonal.

5. The method of claim 1, wherein each antibody of part (c) is monoclonal.

6. The method of claim 1, wherein the antibodies of part (c) are given simultaneously, or consecutively.

7. The method of claim 6, wherein the antibodies are administered within 1 hour.

8. The method of claim 1, wherein the disease is an immune disease or an autoimmune disease or an inflammatory or an autoinflammatory disease.

9. The method of claim 1, wherein the disease is an inflammasome-mediated disease.

10. The method of claim 1, wherein the disease is an IL-1β related disease.

11. The method of claim 1, wherein the disease is an IL-18 related disease.

12. The method of claim 1, wherein the disease is an IL-1β/IL-18 related disease.

13. The method of claim 8, wherein said disease is age-related macular degeneration (AMD).

14. The method of claim 8, wherein said disease is type 2 diabetes (T2D).

15. The method of claim 8, wherein said disease is inflammatory bowel disease (IBD).

16. The method of claim 15, wherein said IBD is Crohn's disease (CD).

17. The method of claim 15, wherein said IBD is ulcerative colitis (UC).

18. The method of claim 1, wherein the patient has not responded to anti-TNF therapy.

19. A method of treating disease in a patient, the method comprising administering to said patient an effective amount of a monoclonal antibody that binds IL-1β and a monoclonal antibody that binds IL-18.

20. A method of neutralizing or blocking IL-1β and/or IL-18 activity in cells or tissue, the method comprising contacting said cells or tissue with a monoclonal antibody that binds IL-1β and a monoclonal antibody that binds IL-18, and thereby neutralizing or blocking said activity.

21. The method of claim 19, wherein said monoclonal antibody that binds IL-1β and said monoclonal antibody that binds IL-18 are administered concurrently or consecutively.

22. The method of claim 20, wherein said cells are contacted concurrently or consecutively with said monoclonal antibody that binds IL-1β and said monoclonal antibody that binds IL-18.

23. An antibody that neutralizes or blocks IL-1β and IL-18 activity.

24. An antibody according to claim 1, wherein the antibody is a bispecific antibody.

25. An antibody according to claim 1, wherein the antibody is humanized.

26. An antibody according to claim 1, wherein the antibody binds to IL-1β and IL-18.
